SEQUENCE LISTING (69 sequences)

SEQ ID NO: 1 - DNA, 20 bp, Homo sapiens
  source 1..20: mol_type = genomic DNA
tgtagactgccaaagtgtat

SEQ ID NO: 2 - DNA, 25 bp, Homo sapiens
  source 1..25: mol_type = genomic DNA
cctggtagactgcatgttagacgag

SEQ ID NO: 3 - DNA, 25 bp, Homo sapiens
  source 1..25: mol_type = genomic DNA
agcccctttcctatctctgtatccc

SEQ ID NO: 4 - DNA, 20 bp, Homo sapiens
  source 1..20: mol_type = genomic DNA
gggttccattacggccagcg

SEQ ID NO: 5 - DNA, 20 bp, Homo sapiens
  source 1..20: mol_type = genomic DNA
gtcctttgctggctgtggag

SEQ ID NO: 6 - DNA, 20 bp, Homo sapiens
  source 1..20: mol_type = genomic DNA
gttggagtccagacggaagc

SEQ ID NO: 7 - DNA, 21 bp, Homo sapiens
  source 1..21: mol_type = genomic DNA
ctgcttctgcgtacaaagggc

SEQ ID NO: 8 - DNA, 22 bp, Homo sapiens
  source 1..22: mol_type = genomic DNA
ggactaactgagcccatggctg

SEQ ID NO: 9 - DNA, 489 bp, Homo sapiens
  source 1..489: mol_type = genomic DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 10 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 114 aa, Homo sapiens
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 12 - DNA, 804 bp, Homo sapiens
  source 1..804: mol_type = genomic DNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatga

SEQ ID NO: 13 - DNA, 231 bp, Homo sapiens
  source 1..231: mol_type = genomic DNA
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccc

SEQ ID NO: 14 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 15 - DNA, 66 bp, Homo sapiens
  source 1..66: mol_type = genomic DNA
atggattttcaggtgcagattttcagcttcctgctaatcagtgcctcagtcataatgtccagagga

SEQ ID NO: 16 - protein, 22 aa, Homo sapiens
MDFQVQIFSFLLISASVIMSRG

SEQ ID NO: 17 - DNA, 75 bp, Homo sapiens
  source 1..75: mol_type = genomic DNA
ggagggggtggtagtgggggtggtgggagtggaggcggaggaagtggcggcgggggcagcggagggggtggtagt

SEQ ID NO: 18 - protein, 25 aa, Homo sapiens
GGGGSGGGGSGGGGSGGGGSGGGGS

SEQ ID NO: 19 - DNA, 39 bp, Homo sapiens
  source 1..39: mol_type = genomic DNA
ggaggaggtggctccggcggaggttccgggggtggctcc

SEQ ID NO: 20 - protein, 13 aa, Homo sapiens
GGGGSGGGSGGGS

SEQ ID NO: 21 - DNA, 198 bp, Homo sapiens
  source 1..198: mol_type = genomic DNA
tcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtga

SEQ ID NO: 22 - protein, 65 aa, Homo sapiens
SSQPTIPIVGIIAGLVLLGAVITGAVVAAVMWRRKSSDRKGGSYTQAASSDSAQGSDVSLTACKV

SEQ ID NO: 23 - DNA, 951 bp, Homo sapiens
  source 1..951: mol_type = genomic DNA
atggattttcaggtgcagattttcagcttcctgctaatcagtgcctcagtcataatgtccagaggaaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacgacagtttgtcttctaatgggaatgtaacagaatctggatgcaaagaatgtgaggaactggaggaaaaaaatattaaagaatttttgcagagttttgtacatattgtccaaatgttcatcaacacttctggagggggtggtagtgggggtggtgggagtggaggcggaggaagtggcggcgggggcagcggagggggtggtagtatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggaggaggtggctccggcggaggttccgggggtggctcctcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtga

SEQ ID NO: 24 - DNA, 951 bp, Homo sapiens
  source 1..951: mol_type = genomic DNA
atggatttccaggtgcaaattttcagtttcctcctcatctctgcttccgtaattatgtcccggggaaactgggtaaacgtgatttcagatcttaagaagatagaggatttgatccaaagtatgcatattgacgcgacactctatactgaatcagacgttcatcctagttgcaaggttacggctatgaaatgctttctgcttgagctgcaagttataagtctggagtcaggcgatgcttctatacatgatacggtggagaatttgattattctcgcaaacgattctcttagctccaatggtaatgtgacagagagcggttgcaaggagtgtgaagaacttgaggaaaaaaacataaaagagtttctccaaagtttcgtgcacatcgttcaaatgtttataaatacgtcaggaggtggtggaagtggcggcggtggatctgggggaggtggttcaggcggagggggaagcggaggagggggttctatcacttgtccgcccccaatgtccgtggagcacgccgacatctgggtgaagtcttattctctttacagtcgagaaaggtacatctgtaacagtggctttaaacgcaaagctggtaccagttctctgacggaatgtgtgctgaacaaagcgaccaatgtagctcactggacgacgcctagtctcaagtgcattagggatcccgctttggtacatcagaggccggcgccgccaggaggcggaggaagcggaggtggaagcggaggagggtcatcatcacagcccactattccaatcgtcgggataattgcaggcctggtattgcttggggcagtcattactggtgcagtggttgcagctgtgatgtggcgaagaaagagcagcgaccggaaaggcggaagctatactcaagcagcctcttctgactccgcgcaaggctccgatgtctctctcactgcgtgtaaggtatga

SEQ ID NO: 25 - protein, 317 aa, Homo sapiens
  VARIANT 317: note = X can be any amino acid
MDFQVQIFSFLLISASVIMSRGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGGGGSGGGGSITCPPPMSVEHADIWVKSYSLYSRERYICNSGFKRKAGTSSLTECVLNKATNVAHWTTPSLKCIRDPALVHQRPAPPGGGGSGGGSGGGSSSQPTIPIVGIIAGLVLLGAVITGAVVAAVMWRRKSSDRKGGSYTQAASSDSAQGSDVSLTACKVX

SEQ ID NO: 26 - DNA, 951 bp, Homo sapiens
  source 1..951: mol_type = genomic DNA
atggattttcaggtgcagattttcagcttcctgctaatcagtgcctcagtcataatgtccagaggaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggagggggtggtagtgggggtggtgggagtggaggcggaggaagtggcggcgggggcagcggagggggtggtagtaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacgacagtttgtcttctaatgggaatgtaacagaatctggatgcaaagaatgtgaggaactggaggaaaaaaatattaaagaatttttgcagagttttgtacatattgtccaaatgttcatcaacacttctggaggaggtggctccggcggaggttccgggggtggctcctcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtga

SEQ ID NO: 27 - DNA, 951 bp, Homo sapiens
  source 1..951: mol_type = genomic DNA
atggattttcaagtacaaatttttagtttcctgctgatcagcgcctcagtcataatgtccaggggtatcacgtgtccacccccgatgtccgtagagcacgctgatatttgggtaaagtcctactccctttattcaagggagcggtatatctgcaacagtggtttcaaacggaaagcaggaacgtctagcttgacagaatgtgttctcaataaagctacgaatgtcgcccattggaccactccatcactgaagtgcatacgggatccggccctggtacaccaacggcctgctcctcctggtgggggaggctccggagggggaggttctggagggggaggtagcggtggcggagggtctgggggaggcgggtccaactgggtgaacgtcatatcagatttgaagaagatcgaagatctgattcagtctatgcatattgatgccaccctttacacggaaagtgatgttcacccaagttgtaaggtcacagcaatgaaatgctttttgctggaactccaagttatatcccttgagagcggtgatgcgagcatccatgataccgttgaaaatttgattatacttgcaaacgacagtttgagctccaacggaaatgtcaccgagtccggttgtaaagagtgcgaggagctcgaggagaaaaatattaaggaattcctccaatcttttgtacatatcgtacaaatgttcataaataccagtggtgggggtggttccgggggaggctcaggtggcggtagctccagtcaacccacaattccaattgtcggaatcatcgcaggcttggtcttgctgggtgccgtcataacgggggcagttgtcgcggcagtaatgtggcgaagaaaatcatctgaccggaaggggggatcttacactcaagcagcaagttcagactcagcccaagggtctgacgtatccttgacggcatgtaaagtttga

SEQ ID NO: 28 - protein, 317 aa, Homo sapiens
  VARIANT 317: note = X can be any amino acid
MDFQVQIFSFLLISASVIMSRGITCPPPMSVEHADIWVKSYSLYSRERYICNSGFKRKAGTSSLTECVLNKATNVAHWTTPSLKCIRDPALVHQRPAPPGGGGSGGGGSGGGGSGGGGSGGGGSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGSGGGSSSQPTIPIVGIIAGLVLLGAVITGAVVAAVMWRRKSSDRKGGSYTQAASSDSAQGSDVSLTACKVX

SEQ ID NO: 29 - DNA, 729 bp, Homo sapiens
  source 1..729: mol_type = genomic DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcattccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaagggtcgacttccggtagcggcaaatcctctgaaggcaaaggtgaggtgcagctgttggagtctgggggaggcttggtacagcctggggggtccctgagactctcctgtgcagtctctggattcacctttaacagctttgccatgagctgggtccgccaggctccagggaaggggctggagtgggtctcagctattagtggtagtggtggtggcacatactacgcagactccgtgaagggccggttcaccatctccagagacaattccaagaacacgctgtatctgcaaatgaacagcctgagagccgaggacacggccgtatatttctgtgcgaaagataagattttatggtttggggagccggtcttcgactactggggccagggaaccctggtcaccgtctcctca

SEQ ID NO: 30 - protein, 243 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKGSTSGSGKSSEGKGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSS

SEQ ID NO: 31 - DNA, 321 bp, Homo sapiens
  source 1..321: mol_type = genomic DNA
attgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcg

SEQ ID NO: 32 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 33 - DNA, 114 bp, Homo sapiens
  source 1..114: mol_type = genomic DNA
caagtgcgaaaggcagctataaccagctatgagaaatcagatggtgtttacacgggcctgagcaccaggaaccaggagacttacgagactctgaagcatgagaaaccaccacag

SEQ ID NO: 34 - protein, 38 aa, Homo sapiens
QVRKAAITSYEKSDGVYTGLSTRNQETYETLKHEKPPQ

SEQ ID NO: 35 - DNA, 1170 bp, Homo sapiens
  source 1..1170: mol_type = genomic DNA
atggattttcaggtgcagattttcagcttcctgctaatcagtgcctcagtcataatgtccagaggagaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcattccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaagggtcgacttccggtagcggcaaatcctctgaaggcaaaggtgaggtgcagctgttggagtctgggggaggcttggtacagcctggggggtccctgagactctcctgtgcagtctctggattcacctttaacagctttgccatgagctgggtccgccaggctccagggaaggggctggagtgggtctcagctattagtggtagtggtggtggcacatactacgcagactccgtgaagggccggttcaccatctccagagacaattccaagaacacgctgtatctgcaaatgaacagcctgagagccgaggacacggccgtatatttctgtgcgaaagataagattttatggtttggggagccggtcttcgactactggggccagggaaccctggtcaccgtctcctcagtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcccaagtgcgaaaggcagctataaccagctatgagaaatcagatggtgtttacacgggcctgagcaccaggaaccaggagacttacgagactctgaagcatgagaaaccaccacagtag

SEQ ID NO: 36 - DNA, 1170 bp, Homo sapiens
  source 1..1170: mol_type = genomic DNA
atggacttccaagtgcagattttttcatttctgctgatatctgcttcagtaatcatgtctaggggagagatcgtacttacgcaatccccggcaaccttgtctctctcacccggggaaagggcgactctgtcatgcagggcaagccaaagcgtgtctagttaccttgcgtggtatcaacagaagccaggacaagctccccggctcctgatttacgacgcctcaaaccgggccactggtatacccgcgcgattctcaggctccgggtcagggactgactttactctcacaatctcctccctcgaacctgaagactttgcggtctactattgccaacagagaagcaactggccgcctaccttcgggcaaggaactaaagtggagattaagggctccacgtccggaagtgggaaatcttctgaaggtaagggcgaggtacagctgttggagagcggaggtggcctggtacaaccgggaggatctctccgcttgtcctgcgcagtgtcaggcttcaccttcaattcctttgctatgagctgggttagacaagcccccgggaagggtctggaatgggtttcagccatctcaggctcagggggaggaacctactatgcggacagcgtcaaagggaggtttacgatttcaagggacaattctaaaaatactctttaccttcaaatgaactctttgcgggcggaggataccgcagtatacttctgcgcaaaggacaaaatcctctggttcggagaacccgtcttcgattattggggccaggggaccttggttactgtgagctcagttaagggaaagcatctgtgtcccagcccattgttcccggggccgtctaagccattttgggttctcgttgttgtcgggggggtattggcgtgctattcattgcttgtcactgtggcctttataatattttgggtacgatccaagaggagccgccttttgcatagcgactatatgaatatgactccacggagacccggtccaacacgcaagcattatcagccatacgcacctcctcgcgacttcgctgcatacaggtctcaggttcggaaggcggcgattacctcctacgagaaaagcgacggagtgtacacgggattgtctacccgcaaccaggagacctacgaaaccctgaagcatgaaaaaccgccacagtag

SEQ ID NO: 37 - protein, 390 aa, Homo sapiens
  VARIANT 390: note = X can be any amino acid
MDFQVQIFSFLLISASVIMSRGEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKGSTSGSGKSSEGKGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQX

SEQ ID NO: 38 - DNA, 117 bp, Homo sapiens
  source 1..117: mol_type = genomic DNA
attgaagttatgtatcctcctccttacctagacaatgagaagagcaatggaaccattatccatgtgaaagggaaacacctttgtccaagtcccctatttcccggaccttctaagccc

SEQ ID NO: 39 - protein, 39 aa, Homo sapiens
IEVMYPPPYLDNEKSNGTIIHVKGKHLCPSPLFPGPSKP

SEQ ID NO: 40 - DNA, 135 bp, Homo sapiens
  source 1..135: mol_type = genomic DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 41 - protein, 45 aa, Homo sapiens
TTTPAPRPPTPAPTIASQPLSLRPEACRPAAGGAVHTRGLDFACD

SEQ ID NO: 42 - DNA, 81 bp, Homo sapiens
  source 1..81: mol_type = genomic DNA
ttttgggtgctggtggtggttgggggagtcctggcttgctatagcttgctagtaacagtggcctttattattttctgggtg

SEQ ID NO: 43 - protein, 27 aa, Homo sapiens
FWVLVVVGGVLACYSLLVTVAFIIFWV

SEQ ID NO: 44 - DNA, 63 bp, Homo sapiens
  source 1..63: mol_type = genomic DNA
atctacatctgggcgcccttggccgggacttgtggggtccttctcctgtcactggttatcacc

SEQ ID NO: 45 - protein, 21 aa, Homo sapiens
IYIWAPLAGTCGVLLLSLVIT

SEQ ID NO: 46 - DNA, 63 bp, Homo sapiens
  source 1..63: mol_type = genomic DNA
ccattttttttctgctgcttcatcgctgtagccatgggaatccgtttcattattatggtagca

SEQ ID NO: 47 - protein, 21 aa, Homo sapiens
PFFFCCFIAVAMGIRFIIMVA

SEQ ID NO: 48 - DNA, 123 bp, Homo sapiens
  source 1..123: mol_type = genomic DNA
aggagtaagaggagcaggctcctgcacagtgactacatgaacatgactccccgccgccccgggcccacccgcaagcattaccagccctatgccccaccacgcgacttcgcagcctatcgctcg

SEQ ID NO: 49 - protein, 41 aa, Homo sapiens
RSKRSRLLHSDYMNMTPRRPGPTRKHYQPYAPPRDFAAYRS

SEQ ID NO: 50 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 51 - protein, 42 aa, Homo sapiens
KRGRKKLLYIFKQPFMRPVQTTQEEDGCSCRFPEEEEGGCEL

SEQ ID NO: 52 - DNA, 360 bp, Homo sapiens
  source 1..360: mol_type = genomic DNA
tggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 53 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 54 - DNA, 339 bp, Homo sapiens
  source 1..339: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 55 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 56 - DNA, 114 bp, Homo sapiens; identical to SEQ ID NO: 33
  source 1..114: mol_type = genomic DNA

SEQ ID NO: 57 - protein, 38 aa, Homo sapiens; identical to SEQ ID NO: 34

SEQ ID NO: 58 - DNA, 66 bp, Homo sapiens; identical to SEQ ID NO: 15
  source 1..66: mol_type = genomic DNA

SEQ ID NO: 59 - protein, 22 aa, Homo sapiens
MDFQVQIFSFLLISASVIMSRG

SEQ ID NO: 60 - DNA, 1704 bp, Homo sapiens
  source 1..1704: mol_type = genomic DNA
atggattttcaggtacaaattttctcattccttctcatttcagcatccgtaataatgtccagaggcgacatccaaatgactcaatcaccaagttccttgtccgcgtccgttggggatagagtaaccataacctgtagggcatcacaagatgtgaatacggccgtagcgtggtatcaacagaaaccaggaaaggctccaaaactccttatttattccgcgagttttctttacagcggagtcccgagtagattctctggttctcgctctggcactgattttactctgaccataagttcactgcaacctgaagactttgcgacctattactgtcaacagcactacacaacgcctcccacttttggacaaggtactaaagtagagattaaacggaccggatccacaagtgggagcgggaagcctggttctggcgaaggatccgaagttcagctggttgaatccggcggtgggttggtgcaacccggggggagcctgcgcctcagctgtgccgcgagtgggtttaacataaaagatacctacattcactgggtgcgccaagctccgggcaaaggacttgaatgggtcgcgaggatctacccgaccaacggttacacaagatatgcggactccgtaaaagggcgattcacgatatccgctgacacatccaagaacacggcgtacttgcaaatgaattctcttagggccgaggacaccgcagtttattactgtagtcgctggggaggtgatggattttatgcgatggacgtttgggggcaagggacgctggtcacggtttccagtgctgcaaccacaacgccagcaccaagacctcccacacccgctcctaccatcgcttcacaacccctttctctgcgaccagaggcgtgtagacccgccgctgggggcgccgttcacacgaggggcctggacttcgcgtgcgacccctttttcttctgctgctttatagctgtggcgatgggaattcgatttataattatggtggcatggagacggaagcggaaggagaaacagtccgagactagcccgaaggagttcttgaccatttatgaagacgtaaaagatttgaagacccggcgcaatcacgaacaagaacaaacgtttccaggaggcggtagtactatatactcaatgattcaaagtcaatcttcagcaccgacttctcaagaacccgcatatactctctatagcctgattcaaccctcacggaagtcagggagcaggaaaaggaaccattcaccgagttttaattccacgatttacgaggtgataggcaagagccagcctaaggcccagaacccggcaagattgtcccgaaaggaactcgaaaactttgatgtgtactctagggtgaaattcagtcggtcagcagatgcccctgcatatcaacaaggacaaaaccaactgtataacgagctcaatcttggtcgacgcgaggaatacgacgtacttgacaagagaagaggaagagaccccgagatggggggcaagccgcagcgaagaaagaatccacaagaagggctctacaatgaactgcagaaggacaaaatggccgaagcctattccgagatcggaatgaagggtgagcgaaggcgaggaaaggggcacgacggcctttaccagggtctgtcaaccgctacgaaagacacgtacgacgccctgcacatgcaagcgctcccaccacgatag

SEQ ID NO: 61 - protein, 567 aa, Homo sapiens
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAATTTPAPRPPTPAPTIASQPLSLRPEACRPAAGGAVHTRGLDFACDPFFFCCFIAVAMGIRFIIMVAWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 62 - DNA, 1470 bp, Homo sapiens
  source 1..1470: mol_type = genomic DNA
atggacttccaagtgcagatattctcctttcttcttatttccgcatccgttataatgagcaggggggatatacaaatgactcagtctccgtcctctctgagtgcatccgtgggcgaccgagtaactatcacgtgccgggcatcccaagatgtaaacacagccgtagcttggtaccaacaaaaaccaggtaaagctcctaaattgcttatttactctgcaagtttcttgtacagtggggtgccgtcccggtttagtggctccagaagcggcactgatttcactctgacaatatcttctcttcaacccgaggattttgctacatattattgccaacaacattatacgactccccctactttcggccaagggactaaagtcgagatcaaacgaaccggttccacgtccgggtcaggtaaacctggttcaggagaaggatcagaggtgcaattggtcgagagtggcgggggattggtacaaccaggtggcagtcttaggctgtcttgtgcagcctcaggctttaacattaaggacacgtacatacactgggtccgacaagcaccgggcaagggtctggaatgggtggcacggatttatcctacaaacggatatactcgatacgcggattcagtcaaggggcgctttaccataagtgccgacactagcaagaacactgcctatcttcagatgaattcattgagggcggaagacactgcggtatactattgctctagatggggaggagacggcttttatgcaatggatgtgtggggtcagggaaccctcgttaccgtatcttctgctgcaaccaccacccccgccccccgaccaccgacaccagcaccaacaatcgcatcccagcctttgtcattgagaccagaggcgtgtagacccgcagctggaggcgcagtccatacgcgggggctggattttgcctgcgacatatatatatgggctcctctcgcgggtacgtgcggtgttttgctcctgtcactcgtgataacaaagcgaggccggaaaaaattgctctatatcttcaaacagccgttcatgcgaccggtgcagacaacacaagaagaagacggctgcagctgcagattccctgaggaggaagaaggtgggtgtgaattgagggttaaattctcccgaagcgcggatgcaccggcgtatcagcagggccaaaatcaactctacaacgagctcaacctggggcggcgggaagagtacgacgtactggacaagcgccgaggcagagaccctgagatggggggcaagcctcaaaggcgaaagaaccctcaggaggggctttacaacgagctccaaaaggacaagatggcggaggcctattcagaaatcggcatgaaaggcgaacgaaggagaggtaaaggacacgacgggctgtatcagggtttgagtactgcaacaaaggatacctacgacgctctccacatgcaagcgctgcccccaaggtag

SEQ ID NO: 63 - protein, 489 aa, Homo sapiens
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAATTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 64 - DNA, 1464 bp, Homo sapiens
  source 1..1464: mol_type = genomic DNA
atggattttcaggtacagattttctcttttcttcttataagcgcgtctgtgatcatgagtcggggcgatatacagatgacccaaagcccttcctcactgtcagcgagcgtaggagatagggtgaccatcacatgcagggcgagccaagatgtgaacacagccgtagcttggtatcagcaaaagccaggcaaggctcccaaactgctgatatattctgctagctttctgtattccggtgtacccagcaggttcagcggctccagaagtggaaccgacttcactctgacaatcagtagccttcaaccagaagatttcgcaacgtactactgtcaacaacattacacgacgcccccaaccttcgggcaaggaacgaaagttgagataaaaaggaccggcagcacctccggaagcgggaagccaggatccggggagggttccgaggtgcaactcgtcgagtcaggtggcggtctcgtgcaaccaggaggctccctgcggctctcttgtgcggctagtggatttaatataaaagatacctatattcactgggtgcgccaagcacctggaaaagggctggagtgggtcgccaggatatatccgacaaatggatacacacggtatgcggacagtgttaaaggcaggttcacaattagcgcagacacgagcaagaatacagcctatcttcagatgaattctctcagggctgaagatactgcagtctactattgctctaggtggggtggtgacggcttttacgctatggatgtctgggggcagggcactctggttactgtcagctctgcgatagaagtcatgtaccctccgccgtatcttgacaatgagaagtctaatgggacaatcatacacgtgaaaggcaagcacttgtgcccctctcccctgttccccggccctagtaaaccgttctgggtgctcgtagtggtcggtggagttcttgcctgttatagtttgttggtaaccgtcgcgtttataatattctgggtccggtccaagagaagccgcctcctgcattccgattacatgaacatgaccccacggaggcccggccctacacggaagcattaccagccatacgctccgcctcgagattttgctgcttataggtcacgagtaaagtttagtagatccgctgacgcccctgcctaccagcagggtcagaatcagctctacaatgaactgaacttgggcaggcgagaagagtatgacgttctcgacaagcgaagggggagagatcccgaaatgggtggtaaaccacaaagacgcaagaatcctcaggaaggattgtacaacgagctccaaaaggataagatggcggaagcatattccgaaattgggatgaagggcgagcgaaggcggggcaaaggacacgacggcctttatcaaggactgtctacggctactaaagacacttatgatgcgctgcacatgcaagcattgccgccgagatag

SEQ ID NO: 65 - protein, 487 aa, Homo sapiens
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 66 - DNA, 1701 bp, Homo sapiens
  source 1..1701: mol_type = genomic DNA
atggatttccaagtccaaatctttagttttttgcttatttcagcctccgtcattatgagtagaggggatatacaaatgacccagtcccctagtagcctgagcgcaagcgttggagaccgggtaaccataacatgtagggctagccaggacgttaatacagccgttgcgtggtatcagcaaaaaccgggcaaagcacctaaactcctgatctatagcgcctcattcctgtatagcggcgtccctagtcgattctcaggaagtagatctggtactgacttcacgttgaccatatcctcactccaacccgaggacttcgcaacatattattgccagcagcactataccacgccgccgacgttcgggcaagggaccaaagtcgaaattaagagaacagggtctacgagtggcagtggtaaacccggttccggcgaggggtccgaagtgcaattggtggaatctggagggggtctcgtacaaccggggggctcccttagactcagttgtgccgcgagcggcttcaatattaaggacacttatatccattgggttcggcaagctcccggcaagggtcttgaatgggtagcacgcatatacccgacaaatggatacacgcggtacgcggatagtgtcaagggtaggtttaccatatctgcggacacgtccaaaaacaccgcctaccttcagatgaattccctccgcgctgaagacactgcggtgtattactgcagccgctggggcggggatgggttttacgccatggatgtgtggggtcagggtacactcgtaactgtgagcagcgccaccacgacgcccgcaccgaggccaccgactccagcacccactatcgcttctcaacctctgtcactgcgccctgaagcctgtcggcctgcggccgggggagcggttcatacgcgggggctggatttcgcttgcgacatttatatatgggcacccctcgcgggtacatgtggcgtcctcttgctgagtctcgtaattacgtggagacgaaagaggaaagagaagcagtctgaaactagccctaaggagttccttactatatatgaggatgttaaagatctgaaaacccgaagaaaccatgaacaggaacaaactttccctggcgggggaagtacgatttacagcatgatccaatctcagtcaagtgcgccaaccagtcaagaacctgcgtatacattgtattccctcattcagccatctaggaaaagcggttcacgcaaaaggaaccatagcccttctttcaattcaaccatctatgaagttattggtaaaagtcaacctaaagcgcaaaatccggcgcgacttagccgcaaagaactcgaaaacttcgatgtatacagtcgggtaaaattctctcgcagtgccgatgcgcctgcctaccagcagggacagaatcagttgtacaacgaactgaacctcggccgaagagaggagtatgatgttcttgataagcggaggggcagagatcccgagatgggtgggaagccacaaagacgaaaaaatcctcaggagggactgtacaatgaacttcaaaaagacaagatggctgaagcctactctgagattgggatgaaaggtgagcgccgaaggggaaagggccacgacggcttgtatcaaggactgtccaccgccactaaagatacgtacgatgccttgcatatgcaagcccttcctccacgctag

SEQ ID NO: 67 - protein, 566 aa, Homo sapiens
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSATTTPAPRPPTPAPTIASQPLSLRPEACRPAAGGAVHTRGLDFACDIYIWAPLAGTCGVLLLSLVITWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 68 - DNA, 1002 bp, Homo sapiens
  source 1..1002: mol_type = genomic DNA
atggagggtatttcaatttatacctccgacaattacacggaggaaatgggaagtggagattacgactccatgaaagagccatgctttagggaagagaacgccaactttaataagatctttctcccgacaatttattctattatcttcctcaccggcattgtcggaaatggtcttgttattcttgttatgggctatcaaaagaaacttcgatctatgactgataagtacaggcttcacctcagtgtcgccgatctgttgtttgtaataactcttccgttttgggcggtagatgccgttgctaactggtacttcggtaacttcctttgtaaagctgtacatgtcatttacacagttaatctgtatagcagcgtgctgatactggcgtttatttcactggaccgctacctcgccatcgttcacgccacgaatagtcagcgaccgcggaagctcttggccgagaaggttgtgtacgtgggcgtgtggattcccgctctgctcttgacaattcccgacttcatatttgccaatgtgagcgaagccgatgatcgctacatctgtgatagattctatccgaatgatttgtgggtagtagtttttcaattccaacacatcatggtgggcctgatcttgccgggaatcgtaatcctgagttgttactgcattattatctctaagttgtcccactcaaagggccatcagaagcgcaaggcgttgaagacgactgttatactcatattggcattctttgcctgttggctgccttactatattgggatatctatagattctttcatactcctggagatcataaagcagggatgtgaatttgaaaatactgtacataagtggatttccataactgaagcgcttgcgttctttcattgttgtctgaatccaattctctatgcgtttctgggggcaaaatttaaaacctcagcgcaacatgcactgaccagtgtatcacggggctctagccttaaaatactttccaaaggcaagtaa

SEQ ID NO: 69 - protein, 334 aa, Homo sapiens
  VARIANT 334: note = X can be any amino acid
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKX

(19) United States
(12) Patent Application Publication
(10) Pub. No.: US 2024/0358828 A1
(43) Pub. Date: Oct. 31, 2024

(54) ENGINEERED NK CELLS, METHODS OF THEIR PRODUCTION AND USES THEREOF

(71) Applicant: Gamida Cell Ltd., Ramat Gan (IL)

(72) Inventors: Yona GEFFEN, Jerusalem (IL); Aviad PATO, Jerusalem (IL); Julia RIFMAN, Jerusalem (IL); Astar HAILU, Jerusalem (IL); Ayal HENDEL, Jerusalem (IL); Nimrod BEN HAI, Jerusalem (IL); Nurit BRICKMAN, Jerusalem (IL)

(21) Appl. No.: 18/682,748

(22) PCT Filed: Aug. 5, 2022
(86) PCT No.: PCT/US2022/039588

§ 371 Date: Feb. 9, 2024

Related U.S. Application Data
(60) Provisional application No. 63/231,372, filed on Aug. 10, 2021.

(51) Int. Cl. A61K 39/00; A61P 35/00; C07K 14/715; C07K 14/725; C07K 16/28; C12N 5/0783

(52) U.S. Cl. CPC A61K 39/4613; A61K 39/4631; A61P 35/00; C07K 14/7051; C07K 14/7155; C07K 16/2896; C12N 5/0646; A61K 2239/21; C07K 2317/622; C12N 2510/00

(57) ABSTRACT

A composition and method of ex vivo producing genetically modified natural killer (NK) cells is disclosed. The method comprising: (a) downregulating expression of a gene of interest in a population of NK cells so as to obtain a population of NK cells having been genetically modified to down-regulate a gene of interest; (b) expanding the population of NK cells having been genetically modified to down-regulate a gene of interest so as to obtain ex vivo expanded population of NK cells; and (c) upregulating expression of at least one membrane bound protein in the ex vivo expanded population of NK cells. A method of ex vivo producing natural killer (NK) cells expressing at least one membrane bound protein is also disclosed. Pharmaceutical compositions and methods of treatment are also disclosed.

### CROSS-REFERENCE TO RELATED APPLICATIONS

This application claims priority to, and the benefit of, U.S. Provisional Application No. 63/231,372, filed Aug. 10, 2021, the contents of which are incorporated herein by reference in their entireties.

### FIELD AND BACKGROUND OF THE INVENTION

The present invention, in some embodiments thereof, relates to engineered Natural Killer (NK) cells and, more particularly, but not exclusively, to NK cells modified to lack expression of a gene of interest and concomitantly expressing a membrane bound protein of interest.

NK cells are cytotoxic lymphocytes that constitute a significant component of the innate immune system. These cells have a variety of functions, especially the killing of tumor cells, virus-infected cells, cells undergoing oncogenic transformation, and other abnormal cells in a living body. Unlike T cells. NK cell killing of target cells is non-specific with regard to particular antigens, rather their recognition of target cells is regulated through the balance between activating and inhibitory signals. Killing of targeted cells is typically mediated by cytolytic proteins, including perforin, granzyme B. and/or granulysin.

NK cells have drawn considerable attention in recent years as a promising tool for immunotherapy in patients with various refractory hematological malignancies and solid tumors, however, the full therapeutic potential of NK cell-based immunotherapy has yet to be realized. Results to date from experimental protocols have been limited mostly to partial responses, with marginal efficacy being attributed mainly to the relatively low number of NK cells infused, their short in vivo persistence, and/or their poor functionality in vivo. Therefore, development of ex vivo NK culture methods that both effectively expand the NK population and increase the functionality of adoptively infused NK cells in vivo is fundamental to improving the clinical applicability of NK cell immunotherapy.

According to an aspect of some embodiments of the present invention there is provided a method of ex vivo producing genetically modified natural killer (NK) cells, the method comprising: (a) downregulating expression of a gene of interest in a population of NK cells so as to obtain a population of NK cells having been genetically modified to down-regulate a gene of interest: (b) expanding the population of NK cells having been genetically modified to down-regulate a gene of interest so as to obtain an ex vivo expanded population of NK cells; and (c) upregulating expression of at least one membrane bound protein in the ex vivo expanded population of NK cells, thereby producing the genetically modified NK cells.

According to an aspect of some embodiments of the present invention there is provided a method of ex vivo producing natural killer (NK) cells expressing at least one membrane bound protein, the method comprising: (a) expanding a population of NK cells by a method comprising: (i) culturing the population of NK cells under conditions allowing for cell proliferation, wherein the conditions comprise providing an effective amount of nutrients, serum. IL-15 and nicotinamide; and (ii) supplementing the population of NK cells with an effective amount of fresh nutrients, serum. IL-15 and nicotinamide 5-10 days following step (i) to produce expanded NK cells, so as to obtain an ex vivo expanded population of NK cells, and (b) upregulating expression of at least one membrane bound protein in the ex vivo expanded population of NK cells, thereby producing the NK cells expressing the at least one membrane bound protein.

According to an aspect of some embodiments of the present invention there is provided an isolated population of NK cells obtainable according to the method of some embodiments of the invention.

According to an aspect of some embodiments of the present invention there is provided a pharmaceutical composition comprising the isolated population of NK cells of some embodiments of the invention and a pharmaceutically active carrier.

According to an aspect of some embodiments of the present invention there is provided a method of treating a disease in a subject in need thereof, the method comprising administering to the subject a therapeutically effective amount of the isolated population of NK cells of some embodiments of the invention, thereby treating the subject.

According to an aspect of some embodiments of the present invention there is provided a therapeutically effective amount of the isolated population of NK cells of some embodiments of the invention for use in treating a disease in a subject in need thereof.

According to some embodiments of the invention, the population of NK cells is derived from cord blood, peripheral blood, bone marrow, CD34+ cells or iPSCs.

According to some embodiments of the invention, the population of NK cells are deprived of CD3⁺ cells.

According to some embodiments of the invention, the population of NK cells comprise CD3−CD56⁺ cells.

According to some embodiments of the invention, the downregulating is effected by a gene editing system.

According to some embodiments of the invention, the NK cells are in a culture. According to some embodiments of the invention, the downregulating is affected 24-72 hours from initiation of culture.

According to some embodiments of the invention, the gene of interest comprises a gene whose product effects proliferation and/or survival of the NK cells.

According to some embodiments of the invention, the gene of interest is selected from the group consisting of CISH, TGFβ receptor and CD38.

According to some embodiments of the invention, expanding the population of NK cells is affected under conditions allowing for cell proliferation, wherein the conditions comprise an effective amount of nutrients, serum, growth factors and nicotinamide.

According to some embodiments of the invention, the growth factors comprise at least one growth factor selected from the group consisting of IL-15, IL-2, IL-7, IL-12, IL-21, SCF and FLT3.

According to some embodiments of the invention, the effective amount of the nicotinamide comprises an amount between 1.0 mM to 10 mM.

According to some embodiments of the invention, expanding the population of NK cells is affected in the presence of feeder cells or a feeder layer.

According to some embodiments of the invention, the feeder cells comprise irradiated cells.

According to some embodiments of the invention, the feeder cells comprise T cells or PBMCs.

According to some embodiments of the invention, the conditions allowing for cell proliferation further comprise a CD3 agonist.

According to some embodiments of the invention, the expanding the population of NK cells is affected for 14-16 days.

According to some embodiments of the invention, the upregulating expression of the at least one membrane bound protein is affected on day 12-14 from initiation of culture.

According to some embodiments of the invention, the upregulating expression of the at least one membrane bound protein is affected by mRNA electroporation.

According to some embodiments of the invention, the at least one membrane bound protein is transiently expressed.

According to some embodiments of the invention, the at least one membrane bound protein comprises a protein which effects an anti-disease function or survival of the NK cells in vivo.

According to some embodiments of the invention, the at least one membrane bound protein is selected from the group consisting of IL-15, IL-15R, Receptor Linker IL-15 (RLI) and TLR.

According to some embodiments of the invention, the at least one membrane bound protein comprises a chimeric antigen receptor (CAR) or a transgenic T cell receptor (tg-TCR). According to some embodiments of the invention, the CAR comprises at least one co-stimulatory domain.

According to some embodiments of the invention, the at least one co-stimulatory domain is selected from the group consisting of CD28, 2B4, CD137/4-1BB, CD134/OX40, Lsk, ICOS and DAP10.

According to some embodiments of the invention, the CAR comprises at least one activating domain.

According to some embodiments of the invention, the activating domain comprises a CD35 or ˜FcR-γ.

According to some embodiments of the invention, the CAR comprises at least one of a transmembrane domain and a hinge domain.

According to some embodiments of the invention, the transmembrane domain is selected from a CD8, a CD28 and a NKG2D.

According to some embodiments of the invention, the hinge domain is selected from a CD8 and a CD28.

According to some embodiments of the invention, the CAR comprises an antigen binding domain being an antibody or an antigen-binding fragment.

According to some embodiments of the invention, the antigen-binding fragment is a Fab or a scFv.

According to some embodiments of the invention, the CAR or tg-TCR has antigenic specificity for an antigen selected from the group consisting of a tumor antigen, a viral antigen, a bacterial antigen, a fungal antigen, a protozoa antigen, and a parasite antigen.

According to some embodiments of the invention, the tumor antigen is associated with a solid tumor.

According to some embodiments of the invention, the tumor antigen is associated with a hematologic malignancy.

According to some embodiments of the invention, the CAR or tg-TCR has antigenic specificity for an antigen selected from the group consisting of HER2/Neu, CD38, CD19, CD319/CS1, ROR1, CD20, CD5, CD7, CD22, CD70, CD30, BCMA, CD25, NKG2D ligands, MICA/MICB, carcinoembryonic antigen, alphafetoprotein, CA-125, MUC-1, epithelial tumor antigen, melanoma-associated antigen, mutated p53, mutated ras, ERBB2, folate binding protein, HIV-1 envelope glycoprotein gp120, HIV-1 envelope glycoprotein gp41, GD2. CD123, CD23, CD30, CD56, c-Met, mesothelin, GD3, HERV-K, IL-11Ralpha, kappa chain, lambda chain. CSPG4, ERBB2. WT-1. EGFRVIII, TRAIL/DR4, and/or VEGFR2.

According to some embodiments of the invention, the at least one membrane bound protein comprises co-expression of:
(i) a CAR or a tg-TCR, and
(ii) a cytokine or a receptor which effects the survival of the NK cells in vivo.

According to some embodiments of the invention, when the gene of interest is CISH, the at least one membrane bound protein comprises IL-15.

According to some embodiments of the invention, when the gene of interest is CD38, the at least one membrane bound protein comprises anti-CD38 CAR.

According to some embodiments of the invention, the disease is selected from the group consisting of a malignant disease, a viral disease, a bacterial disease, a fungal disease, a protozoa disease, and a parasite disease.

According to some embodiments of the invention, the malignant disease is a solid tumor or tumor metastasis.

According to some embodiments of the invention, the malignant disease is selected from the group consisting of a breast cancer, an ovarian cancer, a bladder cancer, a pancreatic cancer, a stomach cancer, a lung cancer, a melanoma, a sarcoma, a neuroblastoma, a colon cancer, a colorectal cancer, an esophageal cancer, a synovial cell cancer, a uterus cancer, a glioma and a cervical cancer.

According to some embodiments of the invention, the malignant disease is a hematological malignancy.

According to some embodiments of the invention, the hematological malignancy comprises a leukemia, a lymphoma or multiple myeloma.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWING(S)

The above and further features will be more clearly appreciated from the following detailed description when taken in conjunction with the accompanying drawings.

FIG. 1A is a schematic illustration showing the regulatory role of CIS following IL-15 stimulation in NK cells. Incorporated from Bottino et al., Transl Cancer Res (2016) 5 (Suppl 4): S875-S877.

FIG. 1B is a schematic illustration showing NK cell regulatory checkpoints: IL-15 signaling drives a negative feedback loop by CISH that is producing CIS regulator. Incorporated from Riggan et al., Clinical & Translational Immunology (202 1) e1238.

FIGS. 2A-C illustrate CISH knockout (KO) sequencing results. (FIGS. 2A-C) Human primary NK cells were electroporated with 4 μM RNP complex targeting CISH. DNA from the cells was extracted after seven days in culture. Sanger sequencing was performed and analyzed via TIDE tool for insertion-deletion (INDEL) frequencies. (FIG. 2A) “Old Guide” (CISH 1) refers to a gRNA sequence taken from the public domain (see Palmer et al, bioRxiv Sep. 25, 2020). (FIG. 2B) Guide4 (CISH2) and (FIG. 2C) Guide 10 (CISH3) are unique gRNAs developed by the inventors. Of note, out of three gRNAs, Guide 4 and Guide 10 yielded high editing rates (85% and 82% INDEL frequency, respectively), while the Old guide′ yielded 30% INDEL frequency.

FIGS. 3A-B illustrate that CRISPR KO of CISH gene in NK cells enhances potency by upregulating cytokine production associated with NK cell activation. (FIGS. 3A-B) Potency assay using intracellular staining measured proinflammatory cytokines (INFy and GM-CSF), cytokine expression is shown for co-culture of control NK cells, mock (electro) control or Cish deleted NK cells (using three different RNA-guides, as described in FIGS. 2A-C). Each type of NK cell was co-cultured with K562 (a human erythroleukemic cell line) and Raji (B-cell lymphoma cell line) with or without anti-CD20 (Rituximab, RTX) enhancing CD16-mediated ADCC cytotoxic activity of NK cells. All compared to NK cells cultured alone. Flow cytometry was used to quantify the cytokine expression gated on CD56+NK cells. (FIG. 3A) expression of IFNy and (FIG. 3B) expression of GM-CSF, both detected by intracellular staining (ICS).

FIGS. 4A-C illustrate that CRISPR KO of CISH gene in NK cells enhances their cytotoxicity and ADCC function. (FIGS. 4A-C) Cytotoxic killing assay was performed via the live-cell imaging system IncuCyte S3, allowing collection of real-time data regarding NK activity. Tumor target cells were labeled with CFSE dye and co-cultured with NK cells for 20 hours in a presence of PI (propidium iodide) in the media. Viable cells remained unstained whereas dead cells were detected by overlap of the CFSE fluorescence staining and P1. Percentages of the target dead cells are shown on the curve of control NK cells, mock (electro) control cells and CISH deleted NK cells (using three different RNA-guides, as described in FIGS. 2A-C), each co-cultured with (FIG. 4A) K562 cell line, (FIG. 4B) Raji cell line and (FIG. 4C) Raji cell line together with Rituximab enhancing ADCC killing activity. Of note, RNA guide 4 (CISH2) showed better killing and ADCC as compared to other CISH KO's tested.

FIG. 5A-D are schematic illustrations showing development of IL-15-based fusion protein configurations to improve IL-15 stability and persistence of immunotherapeutic activity. (FIGS. 5A-B). Representative illustration of the signaling (FIG. 5A) following binding and assembling of the membrane-bound complex IL-15-IL-15Ra that is presented in trans to cells expressing the IL-15Rβγ complex (FIG. 5B) (incorporated from Carroll et al., Rheumatology (2008) 47 (9): 1269-1277 and Wikiwand. Interleukin-15). (FIG. 5C) Designed form of IL-15-IL-15-IL15Ra complex that improves immunological activities (incorporated from Hu et al. Scientific Reports (2018) 8:7675). (FIG. 5D) Alternatively designed IL-15 agents that are in a combination with IL15Ra as demonstrated in clinical cancer treatment (incorporated from Waldmann et al., Front. Immunol. (2020) 11:868.

FIGS. 6A-F illustrate schematic representation of the genetic constructs 301.A (FIG. 6A) and 301.B(FIG. 6C), and polypeptide products of membrane bound RLI-301.A (FIG. 6B) and 301.B(FIG. 6D). Full length sequences of membrane bound N72D-RLI GDA-301A (FIG. 6E) and N72D-RLI GDA-301B(FIG. 6F) are presented. For FIGS. 6E-F, blue represents the sequence of the sushi domain, dark blue represents the sequence of the Exon 3 start, green represents the sequence of the leader peptide, dark green represents the sequence of the linker sequence, red represents the sequence of the extracellular domain, orange represent the sequence of the transmembrane domain and yellow represents the sequence of the cytoplasmic domain.

FIG. 7 illustrates that membrane-bound IL-15 mRNA expression increases potency as tested using the CD107a NK cell degranulation marker. The increasing expression of CD107a was determined by flow cytometry analyses following 6 hr culture of control NK cells, mock (electro) control, or those that were electroporated with mRNA IL-15 301.A (IL-15.1A) or 301.B(IL-15.1B). Each type of NK cells were cultured alone or co-cultured with K562 cell line or Raji (with or without Rituximab).

FIG. 8A-C illustrate that membrane bound IL-15 enhances potency of NK cells as illustrated by elevation of proinflammatory cytokines. Intracellular staining of cytokines was performed following 6 hrs co-incubation of control, IL 15.1A or IL-15.1B mRNA electroporated cells, expression of IFNy (FIG. 8A), GM-CSF(FIG. 8B) and TNFα (FIG. 8C) are shown, all detected by flow cytometry analyses. Of note, both IL.15. LA and IL15. 1B showed increase in expression of proinflammatory cytokines INFg, TNFα and GM-CSF, with an advantage to mbIL-15.1b

FIGS. 9A-C illustrate that membrane bound IL-15 increases cytotoxicity function and ADCC killing in NK cells. The enhanced killing activity of IL15.1A or IL-15.1B mRNA electroporated NK cells were tested. (FIG. 9A-B) Cytotoxic killing assay was performed via the live-cell imaging system IncuCyte S3. Tumor target cells were labeled with CFSE dye and co-cultured with NK cells for 20 hrs in a presence of PI in the media. Viable cells remained unstained whereas dead cells were detected by overlap of the CFSE fluorescence staining and PI. Percentages of the target dead cells are shown on the curve of control NK cells, mock (electro) control cells. IL15.1A or IL 15.1B NK cells and control NK cells treated with soluble IL-15 cytokine (a positive control of the assay, allowing determination of the maximal capability of cells to undergo activation following IL-15 administration). Each type of NK cells co-cultured with (FIG. 9A) K562 cell line for 24 hrs and (FIG. 9B) B-cell lymphoma (BL-2) cell line together with Rituximab for 48 hrs. Alternatively, killing assay by flow cytometry was performed to detect the cytotoxic activity of different NK cells co-cultured with RPMI-8226 myeloma cell line (FIG. 9C).

FIG. 10 illustrates the NK CD38 fratricide problem and CRISPR solution.

FIGS. 11A-H illustrate that CD38 KO NK cells are resistant to fratricide in the presence of Daratumumab. (FIGS. 11A-B)-Flow cytometry gate strategy. (FIGS. 11C-E) CD38 expression on NK, Mock, and KO cells, respectively. (FIG. 11F) summary of NK fratricide. (FIGS. 11G-H) Fratricide flow cytometry analysis of NK(FIG. 11G) and CD38 KO NK(FIG. 11H) with or without DARA (Daratumumab).

FIGS. 12A-B illustrate the gene construct and protein structure of anti-CD38 NK CAR (FIG. 12A) and full-length sequence of anti-CD38 CAR (FIG. 12B). For FIG. 12B, red represents the sequence of the hinge domain, blue represents the sequence of the transmembrane domain, and brown represents the sequence of the cytoplasmic domain. FIG. 13 is a schematic illustration showing combined gene editing technique.

FIG. 14 illustrates flow cytometry killing analysis of non- and manipulated-NK cells against multiple cell lines with or without DARA (Daratumumab).

FIG. 15 is a schematic representation of the anti-HER2 CAR genetic constructs.

FIGS. 16A-D are schematic illustrations showing different constructs engineered to express anti-HER2 CAR.

FIG. 17 is a schematic summary of the constructs demonstrating the appearance on the cells.

FIGS. 18A-G illustrate the sandwich flow cytometry method to determined CAR expression on the NK cells. (FIG. 18A) A schematic illustration of NK expressing anti-HER2 CAR and binding Her2 protein, detected by a specific anti-Her2 antibody. (FIGS. 18B-G) Flow cytometry plots representing the specific determination of anti-HER2 CAR expressing on the electroporated cells only. Gate strategy for the staining was performed using a size gating on live cells (FIG. 18B) followed by staining via anti-Her2 antibody on control NK cells (FIG. 18C), electro (mock) control (FIG. 18D) and on NKs electroporated with CAR-B(FIG. 18E), CAR-C(FIG. 18F) and CAR-D (FIG. 18G), per FIG. 17.

FIG. 19 is an illustration of NK cell regulatory checkpoints, with emphasized inhibitory feedback loop mediated via TGFb signaling. Incorporated from Riggan et al., Clinical & Translational Immunology (2021) e1238.

FIG. 20 illustrates NKp30 surface expression.

FIG. 21 illustrates cytotoxic potency in CISH KO NKs and CISH KO/mb-IL-15 NKs.

FIG. 22 illustrates cytotoxic potency in CISH KO NKs and CISH KO/mb-IL-15 NKs.

FIG. 23 illustrates cytotoxic potency in CISH KO NKs and CISH KO/mb-IL-15 NKs.

FIG. 24 illustrates percent killing and potency in CISH KO NKs and CISH KO/mb-IL-15 NKs.

FIG. 25 illustrates CD122 and NKG2A surface expression CISH KO/mb-IL-15 NKs.

FIG. 26 illustrates TIGIT and LAG3 surface expression CISH KO/mb-IL-15 NKs.

FIG. 27 illustrates flow analysis of CD38 KO and CD38 KO/CD38 CAR (GDA601) CD38 surface expression.

FIG. 28 illustrates CISH guide KO strategy and general CISH KO/mb-IL-15 electroporation workflow.

FIG. 29 illustrates mb-IL-15 target cell killing (K562) and non-target killing (PBMCs).

FIG. 30 illustrates CD38 KO and CD38 KO/CD38 CAR potency analysis.

FIG. 31 illustrates CD38 KO and CD38 KO/CD38 CAR potency analysis.

FIG. 32 illustrates CD38 KO and CD38 KO/CD38 CAR killing and fratricide analysis.

### DETAILED DESCRIPTION

The present invention, in some embodiments thereof, relates to engineered Natural Killer (NK) cells and, more particularly, but not exclusively, to NK cells modified to lack expression of a gene of interest and concomitantly expressing a membrane bound protein of interest.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have illustrated that NK cells can be tailored to target specific disease cells of interest while concomitantly having improved properties for an efficient immunotherapy.

As is shown hereinbelow and in the Examples section which follows, the present inventors have produced NK cells with improved properties by ex vivo expanding NK cell populations under culture conditions including nutrients, serum, IL-15 and nicotinamide (see general materials and experimental procedures section, below). In order to further improve the functionality, survival and/or proliferation of the NK cells, the cells were genetically modified prior to expansion thereof, using CRISPR-Cas9 gene editing system, to downregulate the expression of genes whose products negatively regulate the functionality, survival or proliferation of NK cells (e.g. of checkpoints such as CISH, or of CD38 or TGFβ receptor 2, see Examples 1, 3 and 5, respectively). Furthermore, in order to improve the anti-disease function or survival of the NK cells, the expanded NK cells were modified to transiently express, by mRNA electroporation, a membrane bound protein such as receptor linker IL-15 (RLI, see Example 2) or Toll-like receptor 4 (TLR4, see Example 6), or a chimeric antigen receptor (CAR) such as anti-CD38 CAR (see Example 3) or anti-HER2 CAR (see Example 4).

Taken together, the ex vivo produced NK cells of the invention offer the solution of comprising high numbers, having both a high survival and a high functionality (e.g. high cytotoxicity) in vivo, and being engineered to target any a disease cell of interest (e.g. cells of a solid tumor or metastasis, cells of a hematologic tumor, virally infected cell, etc.). Furthermore, the ex vivo produced NK cells of the invention can be engineered for co-administration with any drug of choice, such as with an anti-CD38 antibody, such as Daratumumab (DARA), which would otherwise kill the NK cells.

NK cell fractions of the present disclosure

The present disclosure provides compositions comprising an NK cell fraction comprising a population of nucleated cells.

In some aspects, the population of nucleated cells can comprise at least about 1.0×10⁶, or at least about 5.0×10⁶, or at least about 1.0×10⁷, or at least about 5.0×10⁷, or at least about 1.0×10⁸, or at least about 5.0×10⁸, or at least about 1.0×10⁹, or at least about 5.0×10⁹, or at least about 1.0×10¹⁰, or at least about 5.0×10¹⁰, or at least about 1.0×10¹¹, or at least about 5.0×10¹¹, or at least about 1.0×10¹², or at least about 5.0×10¹² nucleated cells. In some aspects, the population of nucleated cells can comprise at least about at least about 1.0×10⁶ cells. In some aspects, the population of nucleated cells can comprise at least about at least about 17.5×10⁸ cells. In some aspects, the population of nucleated cells can comprise at least about at least about 35×10⁸. In some aspects, the population of nucleated cells can comprise at least about at least about 2.5×10⁹ cells. In some aspects, the population of nucleated cells can comprise at least about at least about 5×10⁹ cells.

In some aspects, at least about 60%, or at least about 65%, or at least about 70%, or at least about 75%, or at least about 80%, or at least about 85%, or at least about 90%, or at least about 95%, or at least about 99% of the cells in the population of nucleated cells are viable. In some aspects, at least about 70% of the cells in the population of nucleated cells are viable.

In some aspects, at least about at least about 60%, or at least about 65%, or at least about 70%, or at least about 75%, or at least about 80%, or at least about 85%, or at least about 90%, or at least about 95%, or at least about 99% of the cells in the population of nucleated cells are CD56+. In some aspects, at least about 70% of cells in the population of nucleated cells are CD56+.

In some aspects, about 80% to about 99%, or about 85% to about 95%, or about 90 to about 95% of the cells in the population of nucleated cells are CD56+. In some aspects, about 90 to about 95% of the cells in the population of nucleated cells are CD56+.

In some aspects, no more than about 0.1%, or no more than about 0.2%, or no more than about 0).3%, or no more than about 0.4%, or no more than about 0.5%, or no more than about 0.6%, or no more than about 0.7%, or no more than about 0.8%, or no more than about 0.9%, or no more than about 1.0% of cells in the population of nucleated cells are CD3+. In some aspects, no more than 0.5% of cells in the population of nucleated cells are CD3+.

In some aspects, about 0.01% to about 0.1%, or about 0.01% to about 0.2%, or about 0.01% to about 0.3%, or about 0.01% to about 0.4%, or about 0.01% to about 0.5%, or about 0.01% to about 0.6%, or about 0.01% to about 0.7%, or about 0.01% to about 0.8%, or about 0.01% to about 0.9%, or about 0.01% to about 1.0% of cells in the population of nucleated cells CD3+. In some aspects, about 0.01% to about 0.5% of the cells in the population of nucleated cells are CD3+.

In some aspects, about 0.1% to about 0.5%, or about 0.2% to about 0.3% of cells in the population of nucleated cells are CD3+. In some aspects, about 0.2% to about 0.3% of cells in the population of nucleated cells are CD3+.

In some aspects, at least about 60%, or at least about 65%, or at least about 70%, or at least about 75%, or at least about 80%, or at least about 85%, or at least about 90%, or at least about 95%, or at least about 99% of the cells in the population of nucleated cells are CD56+/CD3−. In some aspects, at least about 70% of cells in the population of nucleated cells are CD56+/CD3−. In some aspects, at least about 99% of the cells in the population of nucleated cells are CD56+/CD3−.

In some aspects, about 80% to about 99%, or about 85% to about 95%, or about 90 to about 95% of the cells in the population of nucleated cells is CD56+/CD3−. In some aspects, about 90 to about 95% of the cells in the population of nucleated cells is CD56+/CD3−.

In some aspects, no more than about 0.1%, or no more than about 0.2%, or no more than about 0.3%, or no more than about 0.4%, or no more than about 0.5%, or no more than about 0.6%, or no more than about 0.7%, or no more than about 0.8%, or no more than about 0.9%, or no more than about 1.0% of cells in the population of nucleated cells are CD56−/CD3+. In some aspects, no more than 0.5% of cells in the population of nucleated cells are CD56−/CD3+.

In some aspects, about 0.01% to about 0.1%, or about 0.01% to about 0.2%, or about 0.01% to about 0.3%, or about 0.01% to about 0.4%, or about 0.01% to about 0.5%, or about 0.01% to about 0.6%, or about 0.01% to about 0.7%, or about 0.01% to about 0.8%, or about 0.01% to about 0.9%, or about 0.01% to about 1.0% of cells in the population of nucleated cells are CD56−/CD3+. In some aspects, about 0.01% to 0.5% of cells in the population of nucleated cells are CD56−/CD3+.

In some aspects, about 0.1% to about 0.5%, or about 0.2% to about 0.3% of cells in the population of nucleated cells are CD56−/CD3+. In some aspects, about 0.2% to about 0.3% of cells in the population of nucleated cells are CD56−/CD3+

In some aspects, no more than about 5%, or no more than about 10%, or no more than about 15%, or no more than about 20%, or no more than about 25% of cells in the population of nucleated cells are CD19+. In some aspects, no more than about 10% of cells in the population of nucleated cells are CD19+. In some aspects, no more than about 0.7% of the cells in the population of nucleated cells are CD19+.

In some aspects, about 0.01% to about 5%, or about 0.01% about 10%, or about 0.01% to about 15%, or about 0.01% to about 20%, or about 0.01% to about 25% of cells in the population of nucleated cells are CD19+. In some aspects, about 0 01% to about 10% of cells in the population of nucleated cells are CD19+. In some aspects, about 0.01% to about 0.7% of the cells in the population of nucleated cells are CD19+.

In some aspects, about 0.1% to about 5%, or about 0.1% about 10%, or about 0.1% to about 15%, or about 0.1% to about 20%, or about 0.1% to about 25% of cells in the population of nucleated cells are CD19+. In some aspects, about 0.1% to about 10% of cells in the population of nucleated cells are CD19+. In some aspects, about 0.1% to about 0.7% of the cells in the population of nucleated cells are CD19+.

In some aspects, no more than about 5%, or no more than about 10%, or no more than about 15%, or no more than about 20%, or no more than about 25% of cells in the population of nucleated cells are CD14+. In some aspects, no more than about 10% of cells in the population of nucleated cells are CD14+. In some aspects, no more than about 0.05% of the cells in the population of nucleated cells are CD14+.

In some aspects, about 0.01% to about 5%, or about 0.01% to about 10%, or about 0.01% to about 15%, or about 0.01% to about 20%, or about 0.01% to about 25% of cells in the population of nucleated cells are CD14+. In some aspects, about 0.01% to about 10% of cells in the population of nucleated cells are CD14+. In some aspects, about 0.01% to about 0.05% of the cells in the population of nucleated cells are CD14+.

In some aspects, about 0.1% to about 5%, or about 0.1% to about 10%, or about 0.1% to about 15%, or about 0.1% to about 20%, or about 0.1% to about 25% of cells in the population of nucleated cells are CD14+. In some aspects, about 0.1% to about 10% of cells in the population of nucleated cells are CD14+. In some aspects, about 0.1% to about 0.05% of the cells in the population of nucleated cells are CD14+.

In some aspects, no more than about 0.57% of the cells in the population of nucleated cells are LAG3+. In some aspects, no more than about 1% of the cells in the population of nucleated cells are LAG3+. In some aspects, no more than about 2% of the cells in the population of nucleated cells are LAG3+. In some aspects, no more than about 40% of the cells in the population of nucleated cells are LAG3+. In some aspects, no more than about 2.5%, or no more than about 5%, or no more than about 10%, or no more than about 15%, or no more than about 20%, or no more than about 30%, or no more than about 35%, or no more than about 40%, or no more than about 50% of cells in the population of nucleated cells are LAG3+. In some aspects, no more than about 10% of cells in the population of nucleated cells are LAG3+. In some aspects, about 0.5% to about 40% of cells in the population of nucleated cells are LAG3+.

In some aspects, at least about 10% of the cells in the population of nucleated cells are CD122+. In some aspects, at least about 15%, or at least about 20%, or at least about 25%, or at least about 30%, or at least about 35%, or at least about 40%, or at least about 45%, or at least about 50%, or at least about 55%, or at least about 60%, or at least about 70%, or at least about 80%, of the cells in the population of nucleated cells are CD122+.

In some aspects, no more than about 15% of the cells in the population of nucleated cells are NKG2A+. In some aspects, no more than about 10% of the cells in the population of nucleated cells are NKG2A+. In some aspects, no more than about 5%, or no more than about 2.5%, or no more than 1% of the cells in the population of nucleated cells are NKG2A+. In some aspects, no more than about 0.5% of the cells in the population of nucleated cells are NKG2A+.

In some aspects, no more than about 60% of the cells in the population of nucleated cells are NKG2A+. In some aspects, no more than about 50% of the cells in the population of nucleated cells are NKG2A+. In some aspects, no more than about 45%, or no more than about 35%, or no more than 25% of the cells in the population of nucleated cells are NKG2A+. In some aspects, no more than about 10% of the cells in the population of nucleated cells are NKG2A+.

In some aspects, no more than about 80% of the cells in the population of nucleated cells are NKG2A+. In some aspects, no more than about 75% of the cells in the population of nucleated cells are NKG2A+. In some aspects, no more than about 70%, or no more than about 65% of the cells in the population of nucleated cells are NKG2A+.

In some aspects, no more than about 40% of the cells in the population of nucleated cells are TIGIT+. In some aspects, no more than about 30%, or no more than about 35% of the cells in the population of nucleated cells are TIGIT+. In some aspects, no more than about 20% of the cells in the population of nucleated cells are TIGIT+. In some aspects, no more than about 15% of the cells, or no more than about 10%, or no more than about 5%, or no more than about 2.5%, or no more than about 1% in the population of nucleated cells are TIGIT+.

In some aspects, at least about 90% of the cells in the population of nucleated cells are NKp30+. In some aspects, at least about 80%, or at least about 70%, or at least about 65%, or at least about 60%, or at least about 55%, or at least about 50% of the cells in the population of nucleated cells are NKp30+.

In some aspects, at least about 45% of the cells in the population of nucleated cells are NKp30+. In some aspects, at least about 35%, or at least about 25%, or at least about 15%, or at least about 10%, or at least about 5%, or at least about 2.5% of the cells in the population of nucleated cells are NKp30+.

In some embodiments, the cells in the population of nucleated cells comprise a membrane bound receptor or protein. In some embodiments, the membrane bound receptor or protein is one or more of a Receptor Linker IL-15 (mb-IL-15), IL-15, IL-15R. or TLR.

In some embodiments, the cells in the population of nucleated cells comprise a membrane bound receptor or protein and a gene of interest up regulated, down regulated, or knocked out. In some embodiments, the membrane bound receptor or protein is one or more of a Receptor Linker IL-15 (mb-IL-15) and the gene of interest is CISH. In some embodiments, the gene of interest is CISH and is knocked out. In some embodiments, the cells in the population of nucleated cells comprise a mb-IL-15, wherein no more than about 50% of the cells comprised of mb-IL-15 express CISH. In some embodiments, the cells in the population of nucleated cells comprise a mb-IL-15, wherein no more than about 45%, or no more than about 40%, or no more than about 35%, or no more than about 30%, or no more than about 25% of the cells comprised of mb-IL-15 express CISH. In some embodiments, the cells in the population of nucleated cells comprise a mb-IL-15, wherein no more than about 20%, or no more than about 15%, or no more than about 10%, or no more than about 5%, or no more than about 2.5% of the cells comprised of mb-IL-15 express CISH.

Accordingly, in a non-limiting example, the present disclosure provides NK cell fractions comprising a population of nucleated cells, wherein the population comprises at least 1.0×10° nucleated cells, wherein at least about 70% of the cells in the population are viable and express Receptor Linker IL-15, wherein:
at least about 70% of cells in the population are CD56+;
no more than about 0.5% of the cells in the population are CD3+;
no more than about 10% of the cells in the population are CD19+;
no more than about 10% of the cells in the population are CD14+;
no more than about 40% of the cells in the population are LAG3+;
at least about 50% of the cells in the population are CD122+;
no more than about 60% of the cells in the population are NKG2A+;
no more than about 20% of the cells in the population are TIGIT+; and
at least 50% of the cells in the population are NKp30+.

In some embodiments, the Receptor Linker IL-15 is selected from SEQ ID NO: 25 or SEQ ID NO: 28.

In some embodiments, no more than about 25% of the cells that express Receptor

Linker IL-15 also express CISH. In some embodiments, no more than about 20%, or no more than about 15%, or no more than about 10%, or no more than about 5%, or no more than about 2.5%, or no more than about 1%, or no more than about 0.5%, or no more than about. 01% of the cells that express Receptor Linker IL-15 also express CISH.

In some embodiments, no more than about 50% of the cells that express Receptor Linker IL-15 also express CISH. In some embodiments, no more than about 45%, or no more than about 40%, or no more than about 35%, or no more than about 30%, or no more than about 28% of the cells that express Receptor Linker IL-15 also express CISH.

In some aspects, no more than about 15% of the cells in the population of nucleated cells are CD38+. In some aspects, no more than about 10% of the cells in the population of nucleated cells are CD38+. In some aspects, no more than about 5%, or no more than about 2.5%, or no more than 1% of the cells in the population of nucleated cells are CD38+. In some aspects, no more than about 0.5% of the cells in the population of nucleated cells are CD38+. In some aspects, no more than about 0.1% of the cells in the population of nucleated cells are CD38+.

In some aspects, no more than about 30% of the cells in the population of nucleated cells are CD38+. In some aspects, no more than about 25% of the cells in the population of nucleated cells are CD38+. In some aspects, no more than about 20%, or no more than about 17% of the cells in the population of nucleated cells are CD38+.

In some embodiments, the cells in the population of nucleated cells comprise an anti-CD-38 chimeric antigen receptor. In some embodiments, the cells in the population of nucleated cells comprise an anti-CD-38 chimeric antigen receptor and comprise a CD-38 knockout.

In some embodiments, no more than about 10% of the cells that express anti-CD-38 chimeric antigen receptor also express CD38+. In some embodiments, no more than about 5%, or no more than 2.5%, or no more than 1%, or no more than .05%, or no more than .01% of the cells that express anti-CD-38 chimeric antigen receptor also express CD38+.

In some embodiments, the CAR comprises an anti-CD38 Fab or scFv. In some embodiments, the CAR comprises one or more of a CD28 or CD8 hinge domain. In some embodiments, the CAR comprises one or more of a CD28, CD8, or NKG2D transmembrane domain. In some embodiments, the CAR comprises one or more of a CD28, 4-1BB. 2B4, CD3zetaR. OX40, Lsk, ICOS, DAP10, and Fc fragment of IgE receptor 1g co-stimulatory domain. In some embodiments, the CAR comprises one or more of a CD35, FcR-γ, and Fc-epsilon-R activation domain. In some embodiments, the CAR further comprises a signal peptide or leader peptide.

In some embodiments, the anti-CD-38 CAR is selected from SEQ ID NO: 31 and SEQ ID NO: 32.

In some embodiments, at least about 70% of the cells in the population are viable and express at least one membrane bound receptor and at least one CAR receptor.

In some embodiments, the cells in the population of nucleated cells further comprise a chemokine receptor or a mutant chemokine receptor. In some embodiments, the chemokine receptor is CXCR4 or mutant CXCR4. In some embodiments, the mutant CXCR4 is a CXCR4^{R334X} mutant. In some embodiments, the mutant CXCR4 is SEQ ID NO: 69.

The present disclosure also provides a cryopreserved NK cell fraction, comprising any of the NK cell fractions described herein and DMSO. In some aspects, the concentration of DMSO can be about 1% v/v, or about 2% v/v, or about 3% v/v, or about 4% v/v, or about 5% v/v, or about 6% v/v, or about 7% v/v, or about 8% v/v, or about 9% v/v, or about 10% v/v, or about 11% v/v, or about 12% v/v, or about 13% v/v, or about 14% v/v, or about 15% v/v. In some aspects, the concentration of DMSO can be about 10% v/v.

In some aspects, a cryopreserved NK cell fraction can be stable for at least about 1 month, or at least about 2 months, or at least about 3 months, or at least about 4 months, or at least about 5 months, or at least about 6 months, or at least about 7 months, or at least about 9 months, or at least about 10 months. In some aspects, a cryopreserved NK cell fraction can be stable at about −80° C. for at least about 1 month, or at least about 2 months, or at least about 3 months, or at least about 4 months, or at least about 5 months, or at least about 6 months, or at least about 7 months, or at least about 9 months, or at least about 10 months.

### Potency Assays of the Present Disclosure

The present disclosure provides a first potency assay, the assay comprising the steps of:
a) incubating an NK cell fraction of the present disclosure and a plurality of target cells, wherein the plurality of target cells is stained with at least one proliferation stain;
b) determining the cell death percentage in the plurality of target cells.

In some aspects of the first potency assay, the incubation conditions of step (a) can further comprise at least one anti-cancer therapeutic monoclonal antibody.

In some aspects of the first potency assay, the target cells can be K562 cells.

In some aspects of the first potency assay, the target cells can be Raji (CCL-86) cells.

In some aspects of the first potency assay, the target cells can be Raji (CCL-86) cells, and the incubation conditions of step (a) can further comprise rituximab. In some aspects, the rituximab can be present at a concentration of about 1 μg/ml.

In some aspects of the first potency assay, the target cells can be RPMI cells. In some aspects of the first potency assay, the target cells can be RPMI cells.

and the incubation conditions of step (a) can further comprise daratumumab. In some aspects, the daratumumab can be present at a concentration of about 1 μg/ml.

As would be appreciated by the skilled artisan, determining the cell death percentage in the plurality of target cells in step (b) of the first potency assay can be accomplished using any standard technique known in the art for determining cell death percentages. In a non-limiting example, determining the cell death percentage in the plurality of target cells can comprise: i) staining the NK cell fraction and plurality of target cells incubated in step (a) with at least one viability stain: ii) using fluorescent activated cell sorting (FACS) to separate the plurality of target cells from the NK cell fraction; and iii) using the viability stain to determine the cell death percentage in the plurality of target cells sorted in separated in step (ii).

In some aspects, the at least one proliferation stain can be carboxyfluorescein diacetate, succinimidyl ester (CFSE). As would be appreciated by the skilled artisan, any proliferation stain known in the art can be used in the first potency assay, described herein.

In some aspects, the at least one viability stain can be Helix NP™ Blue (also known as Sytox™ Blue). As would be appreciated by the skilled artisan, any proliferation stain known in the art can be used in the first potency assay.

In some aspects, the incubation in step (a) of the first potency assay can be performed at about 37° C.

In some aspects, the incubation in step (a) of the first potency assay can be performed for at least about three hours.

In some aspects, the ratio of the number of cells in the NK cell fraction to the number of cells in the plurality of target cells in step (a) of the first potency assay can be about 2.5:1, or about 3:1, or about 5:1, or about 10:1.

In some aspects, an NK cell fraction of the present disclosure can be characterized in that when the NK cell fraction is tested using first potency assay described above, wherein target cells are K562 cells, the cell death percentage in the target cells is at least 30%.

In some aspects, an NK cell fraction of the present disclosure can be characterized in that when the NK cell fraction is tested using first potency assay described above, wherein target cells are K562 cells, the cell death percentage in the target cells is at least 50%, or at least 60%, or at least 70%, or at least 80%.

In some aspects, an NK cell fraction of the present disclosure can be characterized in that when the NK cell fraction is tested using first potency assay described above, wherein target cells are RPMI cells, the cell death percentage in the target cells is at least 10% at an E: T ratio of 1:1. In some aspects, an NK cell fraction of the present disclosure can be characterized in that when the NK cell fraction is tested using first potency assay described above, wherein target cells are RPMI cells, the cell death percentage in the target cells is at least 25% at an E: T ratio of 5:1.

In some aspects, an NK cell fraction of the present disclosure can be characterized in that when the NK cell fraction is tested using first potency assay described above, wherein target cells are RPMI cells, the cell death percentage in the target cells is at least 40% at an E: T ratio of 5:1. In some aspects, an NK cell fraction of the present disclosure can be characterized in that when the NK cell fraction is tested using first potency assay described above, wherein target cells are RPMI cells, the cell death percentage in the target cells is at least 40% at an E: T ratio of 2.5:1. In some aspects, an NK cell fraction of the present disclosure can be characterized in that when the NK cell fraction is tested using first potency assay described above, wherein target cells are RPMI cells, the cell death percentage in the target cells is at least 30% at an E: T ratio of 1.25:1.

The present disclosure provides a second potency assay, the assay comprising the steps of:
a) incubating an NK cell fraction of the present disclosure and a plurality of target cells, wherein the NK cell fraction is stained with at least one anti-CD107a antibody comprising a detectable label;
b) treating the NK cell fraction and the plurality of target cells incubated in step (a) with one or more protein trafficking inhibitors and further incubating the NK cell fraction and the plurality of target cells;
c) staining the NK cell fraction and plurality of target cells with:
   at least one viability stain;
   at least one anti-CD56 antibody comprising a detectable label
d) fixing the NK cell fraction and the plurality of target cells;
e) permeabilizing the NK cell fraction and the plurality of target cells;
f) staining the NK cell fraction and plurality of target cells with:
   i) at least one anti-IFNy antibody comprising a detectable label;
   ii) at least one anti-TNFα antibody comprising a detectable label;
g) determining at least one of:
   g1) the percentage of viable cells stained with the at least one anti-CD56 antibody that are also stained with the at least one anti-CD107a antibody (i.e. number of CD107a+/CD56+ cells+number of CD56+ cells x 100%);
   g2) the percentage of viable cells stained with the at least one anti-CD56 antibody that are also stained with the at least one anti-IFNy antibody (i.e. number of IFNγ+/CD56+ cells+number of CD56+ cells x 100%); and
   g3) the percentage of viable cells stained with the at least one anti-CD56 antibody that are also stained with the at least one anti-TNFα antibody (i.e. number of TNFα+/CD56+ cells+number of CD56+ cells x 100%).

In some aspects of the second potency assay, the target cells can be K562 cells, RPMI, Raji, or K562 cells.

In some aspects, the at least one viability stain can be Zombie Violet™ Viability Dye. As would be appreciated by the skilled artisan, any proliferation stain known in the art can be used in the first potency assay.

In some aspects, the one or more protein trafficking inhibitors can comprise brefeldin, GolgiStop™ Protein Transport Inhibitor (BD), a combination of brefeldin and GolgiStop™ Protein Transport Inhibitor, or any other protein tracking inhibitors known in the art.

In some aspects of the second potency assay, the further incubation in step (b) is performed at about 37° C.

In some aspects of the second potency assay, the further incubation in step (b) is performed for at least about 37° C.

As would be appreciated by the skilled artisan, determining at least one (g1)-(g3) of step (g) can be accomplished using any standard technique known in the art for determining percentages of cells labeled with antibodies comprising detectable labels, including, but not limited to fluorescent activated cell sorting (FACS).

In some aspects of the second potency assay, step (g) can comprise determining each of (g1)-(g3).

In some aspects, an NK cell fraction of the present disclosure can be characterized in that when the NK cell fraction is tested using second potency assay described above, wherein target cells are K562 cells, the percentage of viable cells stained with the at least one anti-CD56 antibody that are also stained with the at least one anti-CD107a antibody is at least 25% at an E: T of 3:1.

In some aspects, an NK cell fraction of the present disclosure can be characterized in that when the NK cell fraction is tested using first potency assay described above, wherein target cells are Raji cells, the percentage of viable cells stained with the at least one anti-CD56 antibody that are also stained with the at least one anti-CD107a antibody is at least 2.5% at an E: T of 3:1.

In some aspects, an NK cell fraction of the present disclosure can be characterized in that when the NK cell fraction is tested using first potency assay described above, wherein target cells are RPMI cells, the percentage of viable cells stained with the at least one anti-CD56 antibody that are also stained with the at least one anti-CD107a antibody is at least 10% at an E: T of 3:1.

In some aspects, an NK cell fraction of the present disclosure can be characterized in that when the NK cell fraction is tested using first potency assay described above, wherein target cells are RPMI cells, the percentage of viable cells stained with the at least one anti-CD56 antibody that are also stained with the at least one anti-TNFα antibody is at least 10% at an E: T of 3:1.

In some aspects, an NK cell fraction of the present disclosure can be characterized in that when the NK cell fraction is tested using first potency assay described above, wherein target cells are K562 cells, the percentage of viable cells stained with the at least one anti-CD56 antibody that are also stained with the at least one anti-TNFα antibody is at least 25% at an E: T of 3:1.

In some aspects, an NK cell fraction of the present disclosure can be characterized in that when the NK cell fraction is tested using first potency assay described above, wherein target cells are Raji cells, the percentage of viable cells stained with the at least one anti-CD56 antibody that are also stained with the at least one anti-TNFα antibody is at least 5% at an E: T of 3:1.

In some aspects, an NK cell fraction of the present disclosure can be characterized in that when the NK cell fraction is tested using first potency assay described above, wherein target cells are K562 cells, the percentage of viable cells stained with the at least one anti-CD56 antibody that are also stained with the at least one anti-IFNgamma antibody is at least 25% at an E: T of 3:1.

In some aspects, an NK cell fraction of the present disclosure can be characterized in that when the NK cell fraction is tested using first potency assay described above, wherein target cells are Raji cells, the percentage of viable cells stained with the at least one anti-CD56 antibody that are also stained with the at least one anti-IFNgamma antibody is at least 20% at an E: T of 3:1.

In some aspects, an NK cell fraction of the present disclosure can be characterized in that when the NK cell fraction is tested using first potency assay described above, wherein target cells are RPMI cells, the percentage of viable cells stained with the at least one anti-CD56 antibody that are also stained with the at least one anti-IFNgamma antibody is at least 10% at an E: T of 3:1.

In some aspects, an NK cell fraction of the present disclosure can be characterized in that when the NK cell fraction is tested using first potency assay described above, wherein target cells are K562 cells, the percentage of viable cells stained with the at least one anti-CD56 antibody that are also stained with the at least one anti-GM-CSF antibody is at least 4% at an E: T of 3:1.

In some aspects, an NK cell fraction of the present disclosure can be characterized in that when the NK cell fraction is tested using first potency assay described above, wherein target cells are K562 cells, the percentage of viable cells stained with the at least one anti-CD56 antibody that are also stained with the at least one anti-MIP1alpha antibody is at least 50% at an E: T of 3:1.

In some aspects, an NK cell fraction of the present disclosure can be characterized in that when the NK cell fraction is tested using first potency assay described above, wherein target cells are RPMI cells, the percentage of viable cells stained with the at least one anti-CD56 antibody that are also stained with the at least one anti-MIP1alpha antibody is at least 30% at an E: T of 3:1.

In some aspects, an NK cell fraction of the present disclosure can be characterized in that when the NK cell fraction is tested using first potency assay described above, wherein target cells are Raji cells, the percentage of viable cells stained with the at least one anti-CD56 antibody that are also stained with the at least one anti-MIP1alpha antibody is at least 20% at an E: T of 3:1.

In some aspects, an NK cell fraction of the present disclosure can be characterized in that when the NK cell fraction is tested using first potency assay described above, wherein target cells are K562 cells, the percentage of viable cells stained with the at least one anti-CD56 antibody that are also stained with the at least one anti-MIP1beta antibody is at least 50% at an E: T of 3:1.

In some aspects, an NK cell fraction of the present disclosure can be characterized in that when the NK cell fraction is tested using first potency assay described above, wherein target cells are RPMI cells, the percentage of viable cells stained with the at least one anti-CD56 antibody that are also stained with the at least one anti-MIP1beta antibody is at least 25% at an E: T of 3:1.

In some aspects, an NK cell fraction of the present disclosure can be characterized in that when the NK cell fraction is tested using first potency assay described above, wherein target cells are Raji cells, the percentage of viable cells stained with the at least one anti-CD56 antibody that are also stained with the at least one anti-MIP1beta antibody is at least 20% at an E: T of 3:1.

In some aspects, an NK cell fraction of the present disclosure can be characterized in that when the NK cell fraction is tested using first potency assay described above, wherein target cells are RPMI cells, the percentage of viable cells stained with the at least one anti-CD56 antibody that are also stained with the at least one anti-CD107alpha antibody is at least 40% at an E: T of 3:1.

In some aspects, an NK cell fraction of the present disclosure can be characterized in that when the NK cell fraction is tested using first potency assay described above, wherein target cells are RPMI cells, the percentage of viable cells stained with the at least one anti-CD56 antibody that are also stained with the at least one anti-TNFalpha antibody is at least 50% at an E: T of 3:1.

In some aspects, an NK cell fraction of the present disclosure can be characterized in that when the NK cell fraction is tested using first potency assay described above, wherein target cells are RPMI cells, the percentage of viable cells stained with the at least one anti-CD56 antibody that are also stained with the at least one anti-IFNgamma antibody is at least 5% at an E: T of 3:1.

In some aspects, an NK cell fraction of the present disclosure can be characterized in that when the NK cell fraction is tested using first potency assay described above, wherein target cells are RPMI cells, the percentage of viable cells stained with the at least one anti-CD56 antibody that are also stained with the at least one anti-CM-CSF antibody is at least 15% at an E: T of 3:1.

Thus, according to one aspect of the present invention there is provided a method of ex vivo producing genetically modified natural killer (NK) cells, the method comprising:
(a) downregulating expression of a gene of interest in a population of NK cells so as to obtain a population of NK cells having been genetically modified to downregulate a gene of interest;
(b) expanding the population of NK cells having been genetically modified to downregulate a gene of interest so as to obtain an ex vivo expanded population of NK cells; and
(c) upregulating expression of at least one membrane bound protein in the ex vivo expanded population of NK cells, thereby producing the genetically modified NK cells

According to another aspect of the present invention, there is provided a method of ex vivo producing natural killer (NK) cells expressing at least one membrane bound protein, the method comprising:
(a) expanding a population of NK cells by a method comprising:
   (i) culturing a population of NK cells under conditions allowing for cell proliferation, wherein the conditions comprise providing an effective amount of nutrients, serum, IL-15 and nicotinamide; and
   (ii) supplementing the population of NK cells with an effective amount of fresh nutrients, serum, IL-15 and nicotinamide 5-10 days following step (i) to produce expanded NK cells;
   so as to obtain an ex vivo expanded population of NK cells, and
(b) upregulating expression of at least one membrane bound protein in the ex vivo expanded population of NK cells, thereby producing the NK cells expressing the at least one membrane bound protein.

As used herein, the term “natural killer cells” or “NK cells” refers to large granular lymphocytes involved in the innate immune response. Functionally, NK cells exhibit cytolytic activity against a variety of targets via exocytosis of cytoplasmic granules containing a variety of proteins, including perforin, granulysin and granzyme proteases. Killing is triggered in a contact-dependent, non-phagocytotic process which does not require prior sensitization to an antigen.

Human NK cells are characterized by the presence of the cell-surface markers CD16 and CD56, and the absence of the T cell receptor (CD3). Human bone marrow-derived NK cells are further characterized by the CD2+CD16+CD56+CD3 phenotype, further typically containing the T-cell receptor zeta-chain [zeta-TCR], and often characterized by the presence of NKp46, NKp30 or NKp44. Non-NK cells such as NKT cells or CD8NKT possess characteristics and cell-surface markers of both T cells and NK cells (e g, expression of CD3).

In one embodiment, the population of NK cells comprise mature NK cells. As used herein, the term “mature NK cell” is defined as a committed NK cell, having characteristic surface markers and NK cell function, and lacking the potential for further differentiation. As use herein, mature NK cells include, but are not limited to CD56^{bright} cells, which can proliferate and produce abundant CD56^{dim} cells, exhibiting robust cytotoxicity: CD56^{bright}CD94^{high} and CD56^{dim} CD94^{high} cells. Cell surface expression of the CD56, CD3, CD94 and other markers can be determined, for example, by FACS analysis or immunohistological staining techniques.

In another embodiment, the population of NK cells comprise NK progenitor cells, or mixed populations of NK progenitor cells and mature NK cells. As used herein, the term “progenitor” refers to an immature cell capable of dividing and/or undergoing differentiation into one or more mature effector cells. Lymphocyte progenitors include, for example, pluripotent hematopoietic stem cells capable of giving rise to mature cells of the B cell, T cell and NK lineages. In the B cell lineage (that is, in the developmental pathway that gives rise to mature B cells), progenitor cells also include pro-B cells and pre-B cells characterized by immunoglobulin gene rearrangement and expression. In the T and NK cell lineages, progenitor cells also include bone-marrow derived bipotential T/NK cell progenitors [e.g., CD34 (+) CD45RA (hi) CD7 (+) and CD34 (+) CD45RA (hi) Lin (−) CD10 (+) cells], as well as intrathymic progenitor cells, including double negative (with respect to CD4 and CD8) and double positive thymocytes (T cell lineage) and committed NK cell progenitors.

The NK cells of some embodiments of the invention are isolated cells.

The term “isolated” refers to at least partially separated from the natural environment e.g., from a tissue, e.g., from a human body.

The term “population of NK cells” refers to a heterogeneous mixture of NK cells, such as at different stages of maturity, having different signatures, or having different functions.

NK cells of some embodiments of the present invention may be derived from any source which comprises such cells. NK cells are found in many tissues, and can be obtained, for example, from lymph nodes, spleen, liver, lungs, intestines, deciduous and can also be obtained from induced pluripotent stem cells (iPSCs) or embryonic stem cells (ESC). Typically, cord blood, peripheral blood, mobilized peripheral blood and bone marrow (e.g. CD34+ cells), which contain heterogeneous lymphocyte cell populations, are used to provide large numbers of NK cells for research and clinical use.

According to one embodiment. NK cells are obtained from peripheral blood

Any blood collection method may be employed according to the present teachings. For example, a common method for collecting blood fractions is apheresis, in which whole donor blood is separated into blood components (e.g. plasma, leukocytes and erythrocytes), typically by centrifugation, selected components are drawn off for manipulation (e.g. culturing of leukocyte fractions) and the remainder is returned to the donor. Many suitable apheresis devices are commercially available. Typically, apheresis applies to separation of blood components from the peripheral blood of the donor

Lymphocyte fractions, such as “buffy coat” or apheresis units can be processed to enrich or purify or isolate specific defined populations of cells. The terms “purify” and “isolate” do not require absolute purity: rather, these are intended as relative terms. Thus, for example, a purified lymphocyte population is one in which the specified cells are more enriched than such cells are in its source tissue. A preparation of substantially pure lymphocytes can be enriched such that the desired cells (e.g. NK cells) represent at least 10%, 20%, 30%, 40%, 50% or more of the total cells present in the preparation. Methods for enriching, purifying and isolating lymphocytes are well known in the art, and appropriate methods can be selected based on the desired population. For example, lymphocyte enrichment can be performed using commercially available preparations for negatively selecting unwanted cells, such as FICOLL-HYPAQUE™ and other density gradient mediums formulated for the enrichment of whole lymphocytes. T cells or NK cells.

Methods of selection of NK cells from blood, bone marrow, lymphocyte preparations (e.g. apheresis units) or tissue samples are well known in the art (see, for example, U.S. Pat. No. 5,770,387 to Litwin et al., which is incorporated herein in its entirety by reference). Most commonly used are protocols based on isolation and purification of CD56+ cells, usually following mononuclear cell fractionation, and depletion of non-NK cells such as CD3+, CD19+, CD14+, CD34+ and/or CD133+ cells and the like. Combinations of two or more protocols can be employed to provide NK cell populations having greater purity from non-NK contaminants. The purity of the NK cell preparation is of great significance for clinical applications, as non-NK cells, such as T-cells and NKT cells, contribute to antigen-specific reactions such as graft versus host disease (GVHD), compromising the potential benefits of NK cell transplantation. Commercially available kits for isolation of NK cells include one-step procedures (for example, CD56 microbeads and CD56+. CD56+CD16+ isolation kits from Miltenyi Biotec, Auburn CA), and multistep procedures, including depletion, or partial depletion, of CD3+ or depletion with non-NK cell antibodies recognizing and removing T cells (for example, OKT-3), B cells, stem cells, dendritic cells, monocytes, granulocytes and erythroid cells.

Methods for selection of NK cells according to phenotype include, but are not limited to, immunodetection and FACS analysis. In specific embodiments, the NK cell population is depleted of CD3+ cells. CD14+ cells. CD19+ cells, etc. or is selected for CD56+ cells by immunomagnetic selection, for example, using a CliniMACS (LS Column. Miltenyi Biotec).

Thus, in certain embodiments, the NK cell population is selected or enriched for NK cells, and can be a CD3− depleted NK cell fraction.

According to another embodiment, the NK cell population is selected or enriched for NK cells, and can be a CD56+NK cell fraction.

According to one embodiment, the NK cell population comprises CD56+CD16+CD3− cells and/or CD56+CD16-CD3− cells.

In specific embodiments, the population of cells comprising NK cells at the initiation of culture (i.e. before ex vivo expansion) comprise at least 10%, at least 15%, at least 20%, at least 25%, at least 30%, at least 40%, at least 50%, at least 60%, at least 70%, at least 80%, at least 90% or more CD3−/CD56+ cells.

In specific embodiments, the population of cells comprising NK cells at the initiation of culture comprise at least 40%, at least 50%, at least 60%, at least 70%, at least 80%, at least 90% or more CD3−/CD56+ cells.

In some embodiments, the population of cells comprising NK cells at the initiation of culture comprise between 10%-30% CD3−/CD56+ cells, 10%-50% CD3−/CD56+ cells, 20%-40% CD3−/CD56+ cells, 20%-60% CD3−/CD56+ cells, 30%-50% CD3−/CD56+ cells, 30%-70% CD3−/CD56+ cells, 40%-60% CD3−/CD56+ cells. 40%-80% CD3−/CD56+ cells. 50%-70% CD3−/CD56+ cells. 50%-90% CD3−/CD56+ cells. 60%-80% CD3−/CD56+ cells, 60%-100% CD3−/CD56+ cells, 70%-90% CD3−/CD56+ cells, or 80%-100% CD3−/CD56+ cells.

It will be appreciated that at the initiation of culture the population of cells comprising NK cells may comprise residual monocytes. B cells, T cells, dendritic cells and the like, however, these are ablated through the course of ex vivo culture.

In some embodiments, the NK cell population is devoid of erythrocytes. Thus, in some embodiments, prior to or following CD3+/CD14+/CD19+ cell depletion or CD56+ cell selection, the NK cell fraction undergoes red blood cell (RBC) lysis before culturing. In specific embodiments, red blood cell lysis is accomplished using ammonium chloride potassium (ACK) buffer (Gibco. Thermo Fischer Scientific).

According to some embodiments, NK cells can be cultured from fresh cell populations, while other embodiments culture NK cells from stored cell populations (such as cryopreserved and thawed cells) or previously cultured cell populations.

The NK cells of some embodiments of the invention are genetically modified.

The term “genetically modified” refers to cells which are manipulated to express or not express specific genes, markers or peptides or to secrete or not secrete specific peptides (e.g. cytokines), depending on the application needed (e.g. on the disease to be treated).

The genetic modification may result in a permanent or a transient genetic change to the cell.

According to one embodiment, the genetic modification is in a cell genome. Such modifications are typically stable.

According to one embodiment, NK cells are genetically modified by downregulating expression of a gene of interest in a population of NK cells so as to obtain a population of NK cells having been genetically modified to downregulate a gene of interest.

As used herein, the term “gene of interest” refers to a nucleotide sequence that encodes for a desired mRNA or polypeptide. Depending on the context, the gene of interest refers to a deoxyribonucleic acid, e.g., a gene of interest in a DNA template which can be transcribed to an RNA transcript, or a ribonucleic acid, e.g., a gene of interest in an RNA transcript which can be translated to produce the encoded polypeptide of interest in vitro, in vivo or ex vivo.

According to one embodiment the gene of interest encodes for a transcription factor, a transcription repressor, a recruiting protein, a non-coding RNA (e.g., IRNA, RNA, snoRNA, siRNA, miRNA, long ncRNA, etc.), a secreted protein (e.g. a cytokine, a chemokine, a growth factor, a hormone), a membrane protein, a cell surface protein (e.g. a receptor, a marker), an enzyme (e.g. a kinase), a lysosomal-associated protein, a cytolytic protein, and a metalloproteinase.

According to one embodiment, a gene of interest includes, but is not limited to, a gene whose product effects proliferation, survival, functionality e.g. cytokine production (e.g. IFNy) and/or cytotoxic activity, of the NK cells.

According to one embodiment, the gene of interest comprises CISH, TGFβ receptor or CD38

According to a specific embodiment, the gene of interest renders the NK cells more sensitive to IL-15.

According to a specific embodiment, the gene of interest comprises CISH.

As used herein, the term “CISH” refers to the gene encoding the cytokine-inducible SH2-containing protein (CIS) having the gene symbol “CISH”, or for example, GeneBank Accession nos. NP_037456.5 and NP_659508.1 (protein) and NM_013324.7 and NM 145071.4 (mRNA), or homologs thereof.

According to a specific embodiment, the gene of interest comprises TGFβ receptor. As used herein, the term “TGFβ receptor” refers to the gene encoding the transforming growth factor beta receptor 1 having the gene symbol “TGFBR1”, or for example. GeneBank Accession nos. NP_001124388.1, NP_001293139.1 or NP_004603.1 (protein) and NM 001130916.3, NM_001306210.2 or NM_004612.4 (mRNA), or homologs thereof; the gene encoding the transforming growth factor beta receptor 2 having the gene symbol “TGFBR2”, or for example, GeneBank Accession nos. NP_001020018.1 or NP_003233.4 (protein) and NM_001024847.2 or NM_003242.6 (mRNA), or homologs thereof; or the gene encoding the transforming growth factor beta receptor 3 having the gene symbol “TGFBR3”, or for example, GeneBank Accession nos. NP_001 182612.1, NP_001182613.1 or NP_003234.2 (protein) and NM_001 195683.2, NM 001 195684.1 or NM_003243.5 (mRNA), or homologs thereof.

According to a specific embodiment, the gene of interest comprises CD38.

As used herein, the term “CD38” refers to the gene encoding the CD38 molecule having the gene symbol “CD38”, or for example. GeneBank Accession nos. NP_00 1766.2 (protein) and NM_001775.4 (mRNA), or homologs thereof.

As used herein the phrase “downregulating expression” refers to downregulating the expression of a protein product of a gene of interest (e.g. CISH. CD38 or TGFβ receptor) at the genomic and/or the transcript level using a variety of molecules which interfere with transcription (e.g. DNA editing agents) and/or translation (e.g., RNA silencing agents).

Downregulation of expression may be either transient or permanent.

For the same culture conditions the expression is generally expressed in comparison to the expression in a cell of the same species but not contacted with the downregulating agent or contacted with a vehicle control, also referred to as “control”.

According to specific embodiments, downregulating expression refers to the absence of mRNA and/or protein, as detected by RT-PCR or Western blot, respectively.

According to other specific embodiments downregulating expression refers to a decrease in the level of mRNA and/or protein, as detected by RT-PCR or Western blot, respectively The reduction may be by at least a 10%, at least 20%, at least 30%, at least 40%, at least 50%, at least 60%, at least 70%, at least 80%, at least 90%, at least 95% or by at least 99% reduction.

Non-limiting examples of agents capable of downregulating the expression of a gene of interest (e.g. CISH, CD38 or TGFβ receptor) are described in detail herein below.

Following is a description of various non-limiting examples of methods and DNA editing agents used to downregulate expression of a gene of interest on the genomic (DNA) level and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR.

Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs), T-GEE system and CRISPR/Cas system.

Meganucleases±Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo. M T et al. Nature Methods (2012) 9:073-975: U.S. Pat. Nos. 8,304,222:8,021,867:8, 119,381; 8, 124,369; 8, 129,134, 8,133,697:8, 143,015:8.143,016; 8, 148,098: or 8, 163.514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZENs and TALENs±Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al . . . 2011: Mahfouz et al., 2011: Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break (DSB) Repair of these double-stranded breaks through the nonhomologous end-joining (NHEJ) pathway most often results in small deletions or small sequence insertions (Indels). Since each repair made by NHEJ is unique, the use of a single nuclease pair can produce an allelic series with a range of different deletions at the target site.

The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011: Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence). OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May: 30 (5): 460-5: Miller et al. Nat Biotechnol. (2011) 29:143-148: Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through http://www (dot) talendesign (dot) org). TALEN can also be designed and obtained commercially from e.g., Sangamo Biosciences|M (Richmond. CA).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence (gRNA), and a trans-activating crRNA (tracrRNA) (Jinek et al. Science (2012) 337:816-821.).

It was further demonstrated that a synthetic chimeric single guide RNA (sgRNA) composed of a fusion between crRNA and tracrRNA could direct Cas9 to cleave DNA targets that are complementary to the crRNA in vitro. It was also demonstrated that transient expression of Cas9 in conjunction with synthetic sgRNAs can be used to produce targeted double-stranded breaks (DSBs) in a variety of different species (Cho et al., 2013: Cong et al., 2013; DiCarlo et al., 2013; Hwang et al., 2013a,b; Jinek et al., 2013; Mali et al., 2013). The sgRNA (also referred to herein as single guide RNA (sgRNA)) is typically 80-100-nucleotide sequence encoding a combination of the target homologous sequence (crRNA) and the endogenous bacterial RNA that links the crRNA to the Cas9 nuclease (tracrRNA) in a single chimeric transcript.

The CRIPSR/Cas system for genome editing contains two distinct components: a sgRNA and an endonuclease e.g. Cas9, or three distinct components a gRNA, a tracrRNA and an endonuclease e.g. Cas9.

The sgRNA/Cas9 complex or the gRNA/tracrRNA/Cas9 is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the sgRNA/Cas9 complex or of the gRNA/traerRNA/Cas9 localizes the Cas9 to the genomic target sequence so that the Cas9 can cut both strands of the DNA causing a double-strand break (DSB). Just as with ZFNs and TALENs, the double-stranded breaks (DSBs) produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system is coupled with the ability to easily create synthetic sgRNAs or gRNAs. This creates a system that can be readily modified to target modifications at different genomic sites and/or to target different modifications at the same site (e.g. in the CISH, CD38 or TGFβ receptor gene locus). Additionally, protocols have been established which enable simultaneous targeting of multiple genes. The majority of cells carrying the mutation present biallelic mutations in the targeted genes.

However, apparent flexibility in the base-pairing interactions between the sgRNA or the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is mostly repaired by single strand break repair mechanism involving proteins such as but not only. PARP (sensor) and XRCCI/LIG III complex (ligation). However, two proximal, opposite strand nicks introduced by a Cas9 nickase are treated as a double-strand break, in what is often referred to as a ‘double nick’ CRISPR system. A double-nick, which is basically non-parallel DSB, can be repaired like other DSBs by HR or NHEJ depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that are not likely to change the genomic DNA.

Modified versions of the Cas9 enzyme containing two inactive catalytic domains (dead Cas9, or dCas9) have no nuclease activity while still able to bind to DNA based on sgRNA or gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

Alternatively, CRISPR systems may be fused with various effector domains, such as DNA cleavage domains. The DNA cleavage domain can be obtained from any endonuclease or exonuclease. Non-limiting examples of endonucleases from which a DNA cleavage domain can be derived include, but are not limited to, restriction endonucleases and homing endonucleases (see, for example, New England Biolabs Catalog or Belfort et al. (1997) Nucleic Acids Res.). e.g. FokI endonuclease and I-Crel.

Additional Cas endonucleases that can be used to effect DNA editing with gRNA include, but are not limited to, Cas9, Cpf1 (Zetsche et al., 2015, Cell. 163 (3): 759-71), C2c1, C2c2, C2c3 (Shmakov et al., Mol Cell. 2015 Nov. 5; 60 (3): 385-97), CasX and Cpf1/Cas 12a.

There are a number of publicly available tools available to help choose and/or design target sequences as well as lists of bioinformatically determined unique sgRNAs or gRNA for different genes in different species such as, but not limited to, the Feng Zhang lab's Target Finder, The Alex Scier Labs Target Finder (ChopChop), the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, crRNA (gRNA), tracrRNA and a Cas endonuclease (e.g. Cas9) should be expressed or present (e.g., as a ribonucleoprotein complex (RNP)) in a target cell. Alternatively, both sgRNA and a Cas endonuclease (e.g. Cas9), or the gRNA, tracrRNA and a Cas endonuclease (e.g. Cas9), should be expressed or present (e.g., as a ribonucleoprotein complex) in a target cell. The insertion vector can contain all cassettes on a single plasmid or the cassettes are expressed from separate plasmids. CRISPR plasmids are commercially available such as the px330 plasmid from Addgene (Cambridge, Mass.).

According to a specific embodiment, the DNA editing agent comprises a DNA targeting module (e.g., sgRNA).

According to a specific embodiment, the DNA editing agent comprises a nuclease (e.g. an endonuclease) and a DNA targeting module (e.g., sgRNA).

According to a specific embodiment, the DNA editing agent is CRISPR/endonuclease.

According to a specific embodiment, the DNA editing agent is CRISPR/Cas, e.g. sgRNA and Cas9 or a gRNA, tracrRNA and Cas9.

According to a specific embodiment, the DNA editing agent is a RNP complex of sgRNA and Cas9.

Non-limiting examples of sgRNAs that can be used in the present invention comprise a nucleic acid sequence as set forth in Table 2, herein below.

According to a specific embodiment, the RNP complex is introduced into the NK cell by RNP electroporation, using for example, a Nucleofector or BTX-Gemini Twin Wave Electroporator.

Additional DNA editing agents and systems which may be used to down-regulate expression of a gene of interest on the genomic (DNA) level include, but are not limited to, transposons and TFOs. These are discussed briefly below.

Transposon—refers to a mobile genetic element comprising a nucleotide sequence which can move around to different positions within the genome of a single cell. In the process the transposon can cause mutations and/or change the amount of a DNA in the genome of the cell. A number of transposon systems that are able to also transpose in cells e.g. vertebrates have been isolated or designed, such as Sleeping Beauty [Izsvak and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31 (23): 6873-6881]. Generally, DNA transposons translocate from one DNA site to another in a simple, cut-and-paste manner.

Triplex forming oligonuclotides (TFOs)—TFOs can be designed which can recognize and bind to polypurine/polypirimidine regions in double-stranded helical DNA in a sequence-specific manner. These recognition rules are outlined by Maher III, L. J . . . et al., Science, 1989; 245:725-730: Moser. H. E . . . et al., Science. 1987:238:645-630; Beal. P. A., et al, Science, 1992; 251:1360-1363; Cooney, M., et al., Science, 1988:241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was shown that synthetic oligonucleotides can be targeted to specific sequences (see Seidman and Glazer, J Clin Invest (2003) 112:487-94).

In general, the triplex-forming oligonucleotide has the sequence correspondence:

| | | | | | |
|---|---|---|---|---|---|
| oligo | 3′--A | | G | | T |
| duplex | 5′-A | G | | C | T |
| duplex | 3′--T C G A | | | | |

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression.

Additionally, TFOs designed according to the abovementioned principles can induce directed mutagenesis capable of effecting DNA repair, thus providing both downregulation and upregulation of expression of endogenous genes (Seidman and Glazer, J Clin Invest (2003) 112:487-94). Detailed description of the design, synthesis and administration of effective TFOs can be found in U.S. Patent Application Nos. 2003 017068 and 2003 0096980 to Froehler et al. and 2002 0128218 and 2002 0123476 to Emanuele et al. and U.S. Pat. No. 5,721,138 to Lawn.

It will be appreciated that the DNA editing agent can be a mutagen that causes random mutations and the cells exhibiting downregulation of the expression level and/or activity of the gene of interest may be selected

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid: intercalating agents, such as ethidium bromide; alkylating agents, such as bromouracil: transposons; natural and synthetic alkaloids: bromine and derivatives thereof; sodium azide: psoralen (for example, combined with ultraviolet radiation). The mutagen may be a chemical mutagen such as, but not limited to, ICR191, 1,2,7.8-diepoxy-octane (DEO), 5-azaC, N-methyl-N-nitrosoguanidine (MNNG), ethyl methane sulfonate (EMS) or N-ethyl-N-nitrosourea (ENU).

As mentioned, additional agents which can be used to downregulate expression of a gene of interest in NK cells include RNA silencing agents. Exemplary RNA silencing agents include dsRNAs such as siRNAs, miRNAs and shRNAs: antisense RNA (i.e. single stranded RNA); DNAzymes, RNAzymes and MNAzymes.

Regardless of the method employed to downregulate expression of a gene of interest, downregulation is typically affected ex vivo in a population of NK cells in a cell culture (as further discussed below).

According to one embodiment, downregulation is affected 1-7 days, 1-6 days, 1-5 days, 1-4 days, 1-3 days, 1-2 days from initiation of the cell culture.

According to one embodiment, downregulation is affected 12-24 hours. 12-36 hours. 12-48 hours, 24-36 hours, 24-48 hours, 24-60 hours, 24-72 hours, 36-48 hours. 36-60 hours. 36-72 hours, 48-60 hours, 48-72 hours, 48-84 hours, 60-72 hours, 60-84 hours. 60-96 hours, 72-84 hours, 72-96 hours or 72-120 hours from initiation of the cell culture.

According to a specific embodiment, downregulation is affected 24-48 hours from initiation of the cell culture.

According to a specific embodiment, downregulation is affected 24-72 hours from initiation of the cell culture.

According to one embodiment, the method comprises expanding the population of NK cells having been genetically modified to downregulate a gene of interest so as to obtain an ex vivo expanded population of NK cells.

The term “expanded” when relating to a population of NK cells refers to increased numbers of NK cells through ex vivo or in vitro expansion (proliferation) without negatively affecting the viability or functionality of the cells.

According to one embodiment, fold expansion of the NK cells of some embodiments of the invention is between 2 to 12, e.g. between 3 to 11. e.g. between 4 to 10 (i.e. from day 0) to day 14-16 of culture).

Expansion of NK cells is typically affected in an ex vivo cell culture.

Previous studies have demonstrated that NK cells cultured with growth factors and nicotinamide and/or other nicotinamide moiety, for as little as 7 days, or as many as 3 weeks resulted in enhanced, preferential proliferation and/or functionality as compared to cells cultured with cytokines but with less than 0.1 mM nicotinamide and/or other nicotinamide moiety (see PCT Publication WO2011/080740). In preparing a clinically suitable NK cells for immunotherapy, it is desirable to provide significant ex vivo NK cell expansion while retaining therapeutically advantageous functionality of the expanded NK cells, without requiring lengthy culture duration.

According to one embodiment, expansion of NK cells is affected for a period of 7-30 days, 7-25 days, 7-21 days, 7-14 days, 10-24 days, 10-21 days, 10-18 days, 10-15 days, 10-12 days, 12-21 days. 12-18 days, 12-15 days. 14-21 days, 14-18 days, 14-16 days. 14-15 days, 16-21 days, 16-18 days, or 18-21 days.

According to a specific embodiment, expansion of NK cells is affected for a period of 12-18 days.

According to a specific embodiment, expansion of NK cells is affected for a period of 14-16 days.

Ex vivo culturing of NK cells can be effected, according to this aspect of the present invention, by providing NK cells ex vivo with conditions for cell proliferation and ex vivo culturing the NK cells with a nicotinamide moiety, thereby ex vivo expanding the population of NK cells.

As used herein “culturing” includes providing the chemical and physical conditions (e.g., temperature, gas) which are required for NK cell maintenance, as well as nutrients and growth factors. In one embodiment, culturing the NK cells includes providing the NK cells with conditions for NK cell proliferation. Examples of chemical conditions which may support NK cell proliferation include but are not limited to buffers, nutrients, serum, vitamins and antibiotics as well as cytokines and other growth factors which are typically provided in the growth (i.e., culture) medium. In a particular embodiment, conditions for cell proliferation comprise nutrients, serum and cytokine(s). According to a specific embodiment, the growth factors comprise, for example, IL-15, IL-2. IL-7, IL-12, IL-21. SCF and FLT3.

According to one embodiment, conditions allowing for cell proliferation enable the NK cells to double every 1 day, 1.25 day. 1.5 day. 1.75 day, or 2.0 days.

In one embodiment, the NK culture medium includes a minimal essential medium (MEM), such as MEMα (BI, Bet HaEmek, Israel) and serum. In some embodiments, the serum is provided at 2-20%, 5-15% or 5-10% of the culture medium. In specific embodiments, the serum is human serum, provided at 10% of the culture medium. In a particular embodiment, the culture medium is MEMα comprising 10% Human AB Serum (Sigma-Aldrich, St. Louis, MO). Other media suitable for use with the invention include, but are not limited to Glascow's medium (Gibco Carlsbad CA), RPMI medium (Sigma-Aldrich, St Louis MO) or DMEM (Sigma-Aldrich. St Louis MO). It will be noted that many of the culture media contain nicotinamide as a vitamin supplement for example, MEMα (8.19 mM nicotinamide), RPMI (8, 19 pM nicotinamide), DMEM (32.78 pM nicotinamide) and Glascow's medium (16.39 PM nicotinamide), however, the methods of the present invention relate to exogenously added nicotinamide supplementing any nicotinamide and/or nicotinamide moiety included the medium's formula, or that resulting from overall adjustment of medium component concentrations.

According to one embodiment, culturing the NK cells under conditions allowing for cell proliferation comprises providing the cells with nutrients, serum and cytokines. In some embodiments the at least one growth factor includes cytokines and/or chemokines (e.g. IL-15. IL-2, IL-7, IL-12, IL-21, SCF and FLT3). Cytokines and other growth factors are typically provided in concentrations ranging from 0.5-100 ng/ml, or 1.0-80 ng/ml, more typically 5-750 ng/ml, vet more typically 5.0-50 ng/ml (up to 10X such concentrations may be contemplated), and are available commercially, for example, from Perpo Tech, Inc., Rocky Hill, NJ, USA. In one embodiment, conditions allowing for cell proliferation includes providing the cytokine interleukin 15 (IL-15). In specific embodiments, the population of NK cells are cultured with 20 ng/ml IL-15

Further, it will be appreciated in this respect that novel cytokines are continuously discovered, some of which may find uses in the methods of NK cell proliferation of the present invention.

The culture medium typically also comprises antibiotics, such as but not limited to, gentamicin, penicillin or streptomycin.

For applications, in which cells are introduced (or reintroduced) into a human subject, it is often preferable to use serum-free formulations, such as AIM v® serum free medium for lymphocyte culture or MARROWMAX® bone marrow medium. Such medium formulations and supplements are available from commercial sources such as Invitrogen (GIBCO) (Carlsbad, CA, USA). The cultures can be supplemented with amino acids, antibiotics, and/or with cytokines to promote optimal viability, proliferation, functionality and/or and survival.

According to one embodiment, the population of NK cells is cultured with nutrients, serum, a cytokine (e.g. IL-15) and nicotinamide and/or a nicotinamide moiety. As used herein, the term “nicotinamide moiety” refers to nicotinamide as well as to products that are derived from nicotinamide, derivatives, analogs and metabolites thereof, such as, for example, NAD, NADH and NADPH, which are capable of effectively and preferentially enhancing NK cell proliferation and/or activation. Nicotinamide derivatives, analogs and metabolites can be screened and evaluated for their effect on ex vivo NK proliferation in culture by addition to NK cultures maintained as described herein below, addition to functional assays such as killing and motility assays, or in automated screening protocols designed for high-throughput assays well known in the art, and further discussed below.

As used herein, the phrase “nicotinamide analog” refers to any molecule that is known to act similarly to nicotinamide in the abovementioned or similar assays. Representative examples of nicotinamide analogs can include, without limitation, benzamide, nicotinethioamide (the thiol analog of nicotinamide), nicotinic acid and a-amino-3-indolepropionic acid.

The phrase “nicotinamide derivative” further refers to any structural derivative of nicotinamide itself or of an analog of nicotinamide. Examples of such derivatives include, without limitation, substituted benzamides, substituted nicotinamides and nicotinethioamides and N-substituted nicotinamides and nicotinthioamides, 3-acetylpiridine and sodium nicotinate. In one particular embodiment of the invention the nicotinamide moiety is nicotinamide.

Nicotinamide or nicotinamide moiety concentrations suitable for use in some embodiments of the present invention are typically in the range of about 0.5 mM to about 50 mM, about 1.0 mM to about 25 mM, about 1.0 mM to about 15 mM, about 1.0 mM to about 10 mM, about 2.5 mM to about 20 mM, about 2.5 mM to about 10 mM, about 5.0 mM to about 10 mM. Exemplary effective concentrations of nicotinamide can be of about 0.5 mM to about 15 mM. 1.0 mM to about 10.0 mM, typically 2.5, 5.0 or 7.0 mM, based on the effect of these concentrations of nicotinamide on proliferation and NK cell function.

According to specific embodiments of the invention, nicotinamide is provided at a concentration (mM) of about 0.5, about 0.75, about 1.0, about 1.25, about 1.5, about 1.75, about 2.0, about 2.25, about 2.5, about 2.75, about 3.0, about 3.25, about 3.5, about 3.75, about 4.0, about 4.25, about 4.5, about 4.75, about 5.0, about 5.25, about 5.5, about 5.75, about 6.0, about 6.25, about 6.5, about 6.75, about 7.0, about 7.25, about 7.5, about 7.75, about 8.0, about 8.25, about 8.5, about 8.75, about 9.0, about 9.25, about 9.5, about 9.75, about 10.0, about 11.0, about 12.0, about 13.0, about 14.0, about 15.0, about 16.0, about 17.0, about 18.0 or about 20.0 mM. All effective intermediate concentrations are contemplated. In specific embodiments, conditions allowing proliferation comprise between 1.0 to 10.0 mM nicotinamide. In specific embodiments, conditions allowing proliferation comprise 5.0 mM nicotinamide. In other specific embodiments, conditions allowing proliferation comprise 7.0 mM nicotinamide.

Suitable concentrations of the nicotinamide and/or nicotinamide moiety can be determined according to any assay of NK proliferation and/or activity, for example, cell culture or function. Suitable concentration of nicotinamide is a concentration which use thereof in culture “enhances”, or results in a net increase of proliferation and/or function of NK cells in culture, compared to “control” cultures having less than 0.1 mM of the nicotinamide and tested from the same NK cell source (e.g. cord blood, bone marrow or peripheral blood preparation), in the same assay and under similar culture conditions (duration of exposure to nicotinamide, time of exposure to nicotinamide).

In some studies, ex vivo expansion of purified NK cells by culture with nutrients, serum, cytokines and nicotinamide does not require replenishing the medium or manipulation over the culture period, while other studies have advocated culture medium replenishment (“refeeding”) at different intervals during the NK cell culture. In certain embodiments of the present invention, the population of NK cells is “re-fed” during the culture period. Thus, in specific embodiments, expanding NK cells comprises supplementing the population of NK cells with fresh nutrients, serum. IL-15 and nicotinamide 8-10 days following initiation of the ex vivo culture. In some embodiments, supplementing is provided between 4-12 days following initiation of the ex vivo culture, between 5-10 days following initiation of the ex vivo culture, or between 6-9 days following initiation of culturing of the NK cells.

In some embodiments, supplementing (or “refeeding”) the NK cells in a culture does not comprise removing medium from the NK cell culture. In some embodiments, supplementing (or “refeeding”) comprises removing about 30-80%, about 40-70% or about 45-55% of the medium of the NK cell culture, and replacing that with a similar (e.g. equivalent) volume of fresh medium having the same composition and level of nutrients, serum, cytokines (e.g. IL-15) and nicotinamide as the removed medium. In other embodiments, culture volume following refeeding reaches approximately twice the original culture volume at initiation of the NK cell culture (“seeding”).

NK cell populations can be cultured using a variety of methods and devices. Selection of culture apparatus is usually based on the scale and purpose of the culture. Scaling up of cell culture preferably involves the use of dedicated devices. Apparatus for large scale, clinical grade NK cell production is detailed, for example, in Spanholtz et al. (PLOS ONE (2010) 5: e9221) and Sutlu et al. (Cytotherapy (2010), Early Online 1-12). In some embodiments, culturing the NK cells is effected in flasks, at a cell density of 100-4000 X 10⁶ cells per flask. In specific embodiments, culturing the NK cells (e.g. initiation of the ex vivo culture and/or “re-feeding”) is effected in flasks, at a cell density of 200-300 X 10⁶ cells per flask. In certain embodiments, the flasks are flasks comprising a gas-permeable membrane, such as the G-Rex culture device (G-Rex 100M or closed system G-Rex MCS. WolfWilson, St Paul MN).

Seeding the population of NK cells in culture flasks, such as the G-Rex culture device, can be affected at various densities depending on the size and volume of the culture device. A person of skill in the art is capable of making such a determination. According to one embodiment, the population of NK cells are seeded at a density of 0.01×10⁶ cells/ml to 10×10⁶ cells/ml, 0.01×10⁶ cells/ml to 7.5×10⁶ cells/ml, 0.01×10⁶ cells/ml to 5×10⁶ cells/ml, 0.1×10⁶ cells/ml to 10×10⁶ cells/ml. 0.1×10⁶ cells/ml to 7.5×10⁶ cells/ml, 0.1×10⁶ cells/ml to 5×10⁶ cells/ml, 0.1×10⁶ cells/ml to 2.5×10⁶ cells/ml, 0.1×10⁶ cells/ml to 1×10⁶ cells/ml, 0.25×10⁶ cells/ml to 10×10⁶ cells/ml, 0.25×10⁶ cells/ml to 7.5×10⁶ cells/ml, 0.25×10⁶ cells/ml to 5×10⁶ cells/ml. 0.25×10⁶ cells/ml to 2.5×10⁶ cells/ml, or 0.25×10⁶ cells/ml to 1×10⁶ cells/ml. According to a specific embodiment, the population of NK cells are seeded at a density of 0.25×10⁶ cells/ml to 0.5×10⁶ cells/ml, e.g. 0.35×10⁶ cells/ml to 0.4×10⁶ cells/ml.

It will be appreciated that the density of cells in the culture flask increases with proliferation of the cells over the duration of the culture. Thus, in some embodiments, over the course of expansion in culture, the NK cells of the population of NK cells are cultured at a cell density of 10-4000 X 10⁶ cells per flask, 25-4000 X 10⁶ cells per flask, 50-4000 X 10⁶ cells per flask, 100-4000 X 10⁶ cells per flask, 20-3000 X 10⁶ cells per flask. 100-3000 X 10⁶ cells per flask, 200-3000 X 10⁶ cells per flask, 30-2000 X 10⁶ cells per flask. 100-2000 X 10⁶ cells per flask, 300-2000 X 10⁶ cells per flask, 40-1000 X 10⁶ cells per flask, 100-1000 X 10⁶ cells per flask. 400-1000 X 10⁶ cells per flask, 100-800 X 10⁶ cells per flask, 250-800 X 10⁶ cells per flask. 100-600 X 10⁶ cells per flask or 150-500 X 10⁶ cells per flask. In specific embodiments, over the duration of culture in the flasks, the NK cells of the population of NK cells are cultured at a cell density of 100-3000 X 10⁶ cells per flask.

Culturing the NK cells can be effected with or without feeder cells or a feeder cell layer. According to one embodiment, feeder cells comprise T cells or peripheral blood mononuclear cells (PBMCs). According to a specific embodiment, feeder cells comprise irradiated cells (i.e. non-proliferating cells), e.g. irradiated T cells or irradiated peripheral blood mononuclear cells. Irradiation can be affected, for example, at 20-50 Gy (e.g. 20 Gy, 30 Gy, 40 Gy. 50 Gy), 130 KV, 5 mA. According to one embodiment, when feeder cells are used, the ratio of NK cells to feeder cells in the culture may be 1:1, 1:2, 1:3, 2:1 or 3:1. According to a specific embodiment, the ratio of NK cells to feeder cells in the culture is 1:1.

According to a specific embodiment, when T cells or PBMCs (e.g. irradiated T cells or irradiated PBMCs) are used as feeder cells, the culture is further supplemented with a CD3 agonist to stimulate the T-cells in the feeder cell layer to secrete growth factors beneficial for NK cell expansion. CD3 agonists suitable for use with the method of some embodiments of the invention include, but are not limited to, anti-CD3 monoclonal-CD3 agonist antibodies such as OKT-3, mAb 145-2C11. MGA031 and ChAglyCD3.

According to one embodiment, the method comprises upregulating expression of at least one membrane bound protein in the ex vivo expanded population of NK cells.

As used herein the phrase “upregulating expression” refers to increasing the expression of a membrane bound protein on NK cells. The membrane bound protein may be a protein naturally expressed by the NK cells, or a protein not naturally expressed by the NK cells (i.e. exogenous protein).

For the same culture conditions the expression is generally expressed in comparison to the expression in a cell of the same species but not modified to increasing the level of mRNA and/or protein of a membrane bound protein, or contacted with a vehicle control, also referred to as “control”.

According to one embodiment upregulating the expression of a membrane bound protein refers to increasing the level of mRNA and/or protein, as detected by RT-PCR or

Western blot, respectively. The increase may be by at least a 10%, at least 20%, at least 30%, at least 40%, at least 50%, at least 60%, at least 70%, at least 80%, at least 90%, at least 95% or by at least 99% or more.

Upregulation the expression of a membrane bound protein can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with substrates and the like). According to a specific embodiment, upregulation of the expression of a membrane bound protein on NK cells is affected by introducing exogenous nucleic acids (e.g. mRNA) encoding the membrane bound protein into NK cells. Thus, the NK cells of some embodiments of the invention are modified to express the membrane bound protein.

Upregulation of expression may be either transient or permanent. According to a specific embodiment, the expression of a membrane bound protein is transient (i.e. the cells are not genetically modified in their genome for expression of the membrane bound protein) (Pato et al. Clin. Exp. Immunol. 2015 November; 182 (2) 220-9, the contents of which are incorporated by reference herein in their entirety).

The term “membrane bound protein” as used herein refers to a recombinant molecule presented on a NK cell membrane. The membrane bound protein may be a receptor which binds to a ligand (e.g. antigen) and mediates activation (e.g. anti-disease cytotoxic activity or production of inflammatory cytokines) of the NK cell. Alternatively, the membrane bound protein may be a protein associated with survival, proliferation and/or differentiation of NK cells.

The term “antigen” or “Ag” as used herein is defined as a soluble or non-soluble (such as membrane associated) molecule that provokes an immune response. The skilled artisan will understand that any macromolecule, including virtually all proteins or peptides, as well as carbohydrates, lipids and DNA can serve as an antigen. According to some embodiments of the invention, the antigen is associated with a malignant disease, i.e. tumor antigen (e.g., tumor specific antigen or a tumor associated antigen), a viral protein antigen, a bacterial protein antigen, or a fungal protein antigen, as described in further detail herein below.

According to one embodiment, the membrane bound protein comprises IL-15, IL-15R, Receptor Linker IL-15 (RLI) or TLR,

The term “IL-15” as used herein refers to the gene product of the interleukin 15 gene having the gene symbol “IL15”, or for example, GeneBank Accession nos. NP_000576.1 and NP_751915.1 (protein) and NM_000585.5 and NM_172175.3 (mRNA), or homologs thereof.

According to a specific embodiment, the IL-15 comprises an amino acid substitution of the asparagine residue at position 72, located at the end of helix C, with aspartic acid (i.e. N72D substitution).

The term “IL-15 receptor” as used herein refers to the gene product of the interleukin 15 receptor subunit alpha gene having the gene symbol “IL15RA”, or for example. GeneBank Accession nos. NP_001230468.1, NP_001243694.1, NP_002180.1 and NP_751950.2 (protein) and NM_001243539.2, NM_001256765.1, NM_002189.4 and NM_172200.3 (mRNA), or homologs thereof.

The term “Receptor Linker IL-15 (RLI)” refers to a recombinant protein comprising the binding domain of IL-15Ra (i.e. the so-called sushi domain) bound to IL-15 by a flexible linker IL-15. Exemplary RLI which can be used according to some embodiments of the invention are provided in SEQ ID NO: 25 (termed 301.A) and SEQ ID NO: 28 (termed 301.B).

The term “TLR” as used herein refers to the gene product of the toll like receptor 4 gene having the gene symbol “TLR4”, or for example, GeneBank Accession nos. NP_003257.1, NP_612564.1 and NP_612567.1 (protein) and NM_003266.4, NM_138554.5 and NM_138557.3 (mRNA), or homologs thereof. The term “TLR” also refers to the gene product of the toll like receptor 1 gene having the gene symbol “TLR1”, toll like receptor 2 gene having the gene symbol “TLR2”, toll like receptor 3 gene having the gene symbol “TLR3”, toll like receptor 5 gene having the gene symbol “TLR5”, toll like receptor 6 gene having the gene symbol “TLR6”, toll like receptor 7 gene having the gene symbol “TLR7”, toll like receptor 8 gene having the gene symbol “TLR8”, toll like receptor 9 gene having the gene symbol “TLR9”, or toll like receptor 10 gene having the gene symbol “TLR10”.

According to one embodiment, the membrane bound protein comprises a chimeric antigen receptor (CAR) or a transgenic T cell receptor (tg-TCR).

As used herein, the term “transgenic T cell receptor” or “tg-TCR” refers to a recombinant molecule comprising the specificity of a T cell receptor (TCR), i.e. recognition of antigenic peptides (i.e. antigens) presented by major histocompatability complex (MHC) proteins. Typically, the TCR recognizes antigens, i.e. peptides of foreign (e.g. viral) or cellular (e.g. tumor) origins which have been processed by the cell, loaded onto the MHC complex and trafficked to the cell membrane as a peptide-MHC complex.

The tg-TCR of the invention typically comprises two chains (i.e., polypeptide chains), such as, an alpha chain of a T cell receptor (TCR), a beta chain of a TCR, a gamma chain of a TCR, a delta chain of a TCR, or a combination thereof (e.g. αβ chains or γδ chains). The polypeptides of the tg-TCR can comprise any amino acid sequence, provided that the tg-TCR has antigenic specificity and T cell effector functions as described hereinabove. It will be appreciated that antigen specificity is determined by the TCR heterodimer (i.e. by the αβ or γδ chains).

It will be appreciated that each of the two chains is typically composed of two extracellular domains, i.e. the variable (V) region and the constant (C) region.

According to one embodiment, the tg-TCR comprises the variable regions of a TCR. According to a specific embodiment, the tg-TCR comprises the variable regions of α- and β-chains of a TCR. According to another specific embodiment, the tg-TCR comprises the variable regions of γ- and δ-chains of a TCR.

According to some embodiments of the invention, the variable region of the tg-TCR comprises complementarity determining regions (CDRs) which are capable of specifically binding the antigen. The CDRs may be selected from any of CDR1, CDR2, CDR3 and/or CDR4. According to a specific embodiment, the CDRs are present on a single chain, preferably the CDRs are present on both chains of the tg-TCR.

According to one embodiment, the tg-TCR comprises the constant regions of a TCR. According to a specific embodiment, the tg-TCR comprises the constant regions of α- and β-chains of a TCR. According to another specific embodiment, the tg-TCR comprises the constant regions of C- and γ- and δ-chains of a TCR.

The choice of tg-TCR depends upon the type and number of antigens that define the MHC-peptide complex of a target cell. For example, the tg-TCR may be chosen to recognize an MHC-peptide complex on a target cell associated with a particular disease state. Thus, for example, markers that may act as antigens for recognition by the tg-TCR may include those associated with viral, bacterial and parasitic infections and cancer cells. Examples are provided below.

To generate a successful tg-TCR, an appropriate target sequence needs to first be identified. Accordingly, a TCR may be isolated from an antigen reactive T cell (e.g. tumor reactive T cell) or, where this is not possible, alternative technologies can be employed. According to an exemplary embodiment, a transgenic animal (e.g. rabbit or mouse, preferably a human-HLA transgenic mouse) is immunized with human antigen peptides (e.g. tumor or viral antigens) to generate T cells expressing TCRs against the human antigens [as described e.g. in Stanislawski et al., Nat Immunol. (2001) 2 (10): 962-70]. According to another exemplary embodiment, antigen-specific T cells (e.g. tumor specific T cells) are isolated from a patient experiencing disease (e.g. tumor) remission and the reactive TCR sequences are isolated therefrom [as described e.g. in de Witte et al., Blood (2006) 108 (3): 870].

According to another exemplary embodiment, in vitro technologies are employed to alter the sequence of an existing TCR to enhance the avidity of a weakly reactive antigen-specific TCR with a target antigen (such methods are described below).

According to one embodiment, the signaling module of the tg-TCR may comprise a single subunit or a plurality of signaling units. Accordingly, the tg-TCR of the invention may use co-receptors that act in concert with a TCR to initiate signal transduction following antigen receptor engagement, as well as any derivative or variant of thereof having the same functional capability.

According to one embodiment, the TCR signaling module comprises the CD3 complex (e.g. CD3 chains, e.g. CD3δ/ε, CD3γ/ε and/or zeta chains, e.g. ξ/ξ or ξ/η).

Additionally or alternatively, the TCR signaling module may comprise co-stimulatory domains to provide additional signals to the T cell. These are discussed in detail for CAR molecules herein below.

According to one embodiment, the tg-TCR may comprise a transmembrane domain as described in detail for CAR molecules herein below.

As used herein the phrase “chimeric antigen receptor (CAR)” refers to a recombinant molecule which combines specificity for a desired antigen with a T cell receptor-activating intracellular domain (i.e. T cell receptor signaling module) to generate a chimeric protein that exhibits cellular immune activity to the specific antigen. Typically, a CAR recognizes an antigen (e.g. protein or non-protein) expressed on the cell surface (rather than internal antigens) independently of the major histocompatibility complex (MFIC).

Thus, the CAR of the invention generally comprises an extracellular domain comprising an antigen binding moiety, a transmembrane domain and an intracellular domain (i.e. the cytoplasmic domain also referred to as endo-domain) that is required for an efficient response of the T cell to the antigen.

### Antigen Binding Moiety

In one embodiment, the CAR of the invention comprises a target-specific binding element otherwise referred to as an antigen binding moiety. The choice of moiety depends upon the type and number of ligands (i.e. antigens) that define the surface of a target cell. For example, the antigen binding domain may be chosen to recognize a ligand (i.e. antigen) that acts as a cell surface marker on target cells associated with a particular disease state. Thus examples of cell surface markers that may act as ligands for the antigen moiety domain in the CAR of the invention include those associated with viral, bacterial and parasitic infections and cancer cells.

According to some embodiments of the invention, the antigen binding moiety comprises complementarity determining regions (CDRs) which are capable of specifically binding the antigen. Such CDRs can be obtained from an antibody.

The term “antibody” as used in this invention includes intact molecules as well as functional fragments thereof, such as Fab, Fab′, F (ab) 2, Fv, linear antibodies, scFv antibodies, and multispecific antibodies formed from antibody fragments that are capable of binding to the antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain: (2) Fab′, the fragment of an antibody molecule that can be obtained by treating whole antibody with pepsin, followed by reduction, to yield an intact light chain and a portion of the heavy chain: two Fab′ fragments are obtained per antibody molecule: (3) (Fab) 2, the fragment of the antibody that can be obtained by treating whole antibody with the enzyme pepsin without subsequent reduction: F (ab) 2 is a dimer of two Fab″ fragments held together by two disulfide bonds: (4) Fv, defined as a genetically engineered fragment containing the variable region of the light chain and the variable region of the heavy chain expressed as two chains: (5) Single chain antibody (“SCA”), a genetically engineered molecule containing the variable region of the light chain and the variable region of the heavy chain, linked by a suitable polypeptide linker as a genetically fused single chain molecule: (6) CDR peptide is a peptide coding for a single complementarity-determining region (CDR); and (7) Single domain antibodies (also called nanobodies), a genetically engineered single monomeric variable antibody domain which selectively binds to a specific antigen. Nanobodies have a molecular weight of only 12-15 kDa, which is much smaller than a common antibody (150-160 kDa).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa- and lambda-light chains refer to the two major antibody light chain isotypes.

By the term “synthetic antibody” as used herein, is meant an antibody which is generated using recombinant DNA technology, such as, for example, an antibody expressed by a bacteriophage as described herein The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Methods of producing polyclonal and monoclonal antibodies as well as fragments thereof are well known in the art (See for example, Harlow and Lane, Antibodies: A Laboratory Manual. Cold Spring Harbor Laboratory, New York. 1988, incorporated herein by reference)

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F (ab) 2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter. R. R. [Biochem. J. 73:119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2:97-105 (1991): Bird et al., Science 242:423-426 (1988): Pack et al., Bio/Technology 11:1271-77 (1993); and U.S. Pat. No. 4,946,778, which is hereby incorporated by reference in its entirety.

CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example. Larrick and Fry [Methods, 2:106-10 (1991)].

Once the CDRs of an antibody are identified, using conventional genetic engineering techniques, expressible polynucleotides encoding any of the forms or fragments of antibodies described herein can be synthesized and modified in one of many ways in order to produce a spectrum of related-products.

According to some embodiments of the invention, the CDRs are derived from ap T cell receptor (TCR) which specifically binds to the antigen.

According to some embodiments of the invention, the CDRs are derived from γδ T cell receptor (TCR) which specifically binds to the antigen.

According to some embodiments of the invention, the CDRs are derived from an engineered affinity-enhanced ap T cell receptor or γδ T cell receptor (TCR) which specifically binds to the antigen (as discussed in detail herein above).

According to some embodiments of the invention, the CDRs are derived from an engineered αβ T cell receptor or γδ T cell receptor (TCR) with improved stability or any other biophysical property.

According to some embodiments of the invention, the CDRs are derived from a T cell receptor-like (TCRLs) antibody which specifically binds to the antigen. Examples of TCRLs and methods of generating same are described in WO03/068201, WO2008/120203, WO2012/007950, WO2009125395, WO2009/125394, each of which is fully incorporated herein by their entirety.

According to some embodiments of the invention, the antigen binding domain comprises a single chain Fv (scFv) molecule.

### Cytoplasmic Domain

The cytoplasmic domain (also referred to as “intracellular signaling domain” or “T cell receptor signaling module”) of the CAR molecule of the invention is responsible for activation of at least one of the normal effector functions of the cell in which the CAR has been placed in.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR molecule of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Thus, NK cell activation can be mediated by two distinct classes of cytoplasmic signaling sequence: those that initiate antigen-dependent primary activation (primary cytoplasmic signaling sequences) and those that act in an antigen-independent manner to provide a secondary or co-stimulatory signal (secondary cytoplasmic signaling sequences).

Primary cytoplasmic signaling sequences that act in a stimulatory manner may contain signaling motifs which are known as immunoreceptor tyrosine-based activation motifs (ITAMs). Examples of ITAM containing primary cytoplasmic signaling sequences that are of particular use in the invention include those derived from TCR zeta, FeR gamma, FcR beta, CD3 gamma, CD3 delta, CD3 epsilon. CD5, CD22. CD79a. CD79b, and CD66d. It is particularly preferred that cytoplasmic signaling molecule in the CAR of the invention comprises a cytoplasmic signaling sequence derived from CD3 zeta.

The co-stimulatory signaling region typically refers to a portion of the CAR molecule comprising the intracellular domain of a co-stimulatory molecule. Co-stimulatory molecules are cell surface molecules other than antigen receptors or their ligands that are required for an efficient response of lymphocytes to antigen. Co-stimulatory molecules include but are not limited to an MHC class I molecule. BTLA and a Toll ligand receptor. A co-stimulatory ligand can include, but is not limited to, CD7, B7-1 (CD80), B7-2 (CD86), PD-L1, PD-L2, 4-1BBL. ( ) 40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27. CD28, 4-1BB, OX40, CD30, CD40, PD-1, ICOS, lymphocyte function-associated antigen-1 (LFA-1). CD2. CD7, LIGHT. NKG2C, B7-H3, and a ligand that specifically binds with CD83.

According to one embodiment, the cytoplasmic domain of the CAR can be designed to comprise the CD3− zeta signaling domain by itself or combined with any other desired cytoplasmic domain(s) useful in the context of the CAR of the invention. For example, the cytoplasmic domain of the CAR can comprise a CD3 zeta chain portion and a co-stimulatory signaling region. The co-stimulatory signaling region refers to a portion of the CAR comprising the intracellular domain of a co-stimulatory molecule. A co-stimulatory molecule is a cell surface molecule other than an antigen receptor or their ligands that is required for an efficient response of lymphocytes to an antigen. Examples of such molecules include CD27, CD28, 4-1BB (CD137), OX40 (CD134), CD30, CD40, PD-1, DAP 10, 2B4, Lsk, ICOS, lymphocyte function-associated antigen-1 (LFA-1), CD2, CD7, LIGHT, NKG2C, B7-H3, and a ligand that specifically binds with CD83, and the like.

According to some embodiments of the invention, the intracellular domain comprises the CD35-chain [CD247 molecule, also known as “CD3− ZETA” and “CD3z”: GenBank Accession NOs. NP_000725.1 and NP_932170.1], which is the primary transmitter of signals from endogenous TCRs.

According to some embodiments of the invention, the intracellular domain comprises various co-stimulatory protein receptors to the cytoplasmic tail of the CAR to provide additional signals to the T cell (“second generation” CAR). Examples include, but are not limited to. CD28 [e.g., GenBank Accession Nos. NP_001230006.1, NP_001230007.1. NP_006130.1], 4-1BB [tumor necrosis factor receptor superfamily, member 9 (TNFRSF9), also known as “CD137”, e.g, GenBank Accession No. NP_001552.2]. ICOS [inducible T-cell co-stimulator. e.g., GenBank Accession No. NP_036224.1], DAP10 [hematopoietic cell signal transducer. e.g., GenBank Accession Nos. NP_001007470, NP_055081.1], 2B4 | CD244 molecule, e.g. GenBank Accession Nos. NP_001160135.1, NP_001160136.1, NP_057466.1] and Lsk [LCK proto-oncogene. Src family tyrosine kinase. e.g., GenBank Accession Nos. NP 001036236.1, NP_005347.3]. Preclinical studies have indicated that the “second generation of CAR designs improves the antitumor activity of T cells.

According to some embodiments of the invention, the intracellular domain comprises at least one, at least two, at least three or more of the polypeptides selected from the group consisting of: CD35 (CD247. CD3z), CD27, CD28, 4-1BB/CD137, 2B4, ICOS, OX40/CD134, DAP10, tumor necrosis factor receptor (TNFr) and Lsk.

According to some embodiments of the invention, the intracellular domain comprises multiple signaling domains, such as CD3z-CD28-4-1BB or CD3z-CD28-OX40, to further augment potency. The term “OX40” refers to the tumor necrosis factor receptor superfamily, member 4 (TNFRSF4), e.g., GenBank Accession No. NP_003318.1 (“third-generation” CARs).

According to some embodiments of the invention, the intracellular domain comprises CD28-CD3z, CD3z, CD28-CD137-CD3z. The term “CD137” refers to tumor necrosis factor receptor superfamily, member 9 (TNFRSF9), e.g., GenBank Accession No. NP_001552.2.

According to a specific embodiment, the intracellular domain comprises CD3z and CD28.

According to a specific embodiment, the intracellular domain comprises CD3z and 4-1 BB.

According to a specific embodiment, the intracellular domain comprises CD3z and 2B4.

### Transmembrane Domain

The transmembrane domain of the CAR may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of) the alpha, beta or zeta chain of the T-cell receptor, CD28. CD3 epsilon, CD45. CD4, CD5, CD8. CD9. CD16, CD22, CD33, CD37. CD64. CD80, CD86, CD134, CD137, CD154 or NKG2D.

Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain.

According to a specific embodiment, the transmembrane domain comprises CD8.

According to a specific embodiment, the transmembrane domain comprises CD28. According to a specific embodiment, the transmembrane domain comprises NKG2D.

According to some embodiments of the invention, the transmembrane domain comprised in the CAR molecule of some embodiments of the invention is a transmembrane domain that is naturally associated with one of the domains in the CAR. According to some embodiments of the invention, the transmembrane domain can be selected or modified by amino acid substitution to avoid binding of such domains to the transmembrane domains of the same or different surface membrane proteins to minimize interactions with other members of the receptor complex.

According to some embodiments, between the extracellular domain and the transmembrane domain of the CAR molecule, or between the cytoplasmic domain and the transmembrane domain of the CAR molecule, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

Optionally, a short oligo- or polypeptide linker, preferably between 2 and 10 amino acids in length may form the linkage between the transmembrane domain and the cytoplasmic signaling domain of the CAR (also referred to as “hinge”). A glycine-serine doublet provides a particularly suitable linker.

According to a specific embodiment, a hinge region of CD8 is used in construction of the CAR molecule.

According to a specific embodiment, a hinge region of CD28 is used in construction of the CAR molecule.

As mentioned, the CAR or the tg-TCR has antigenic specificity for an antigen selected from the group consisting of a tumor antigen, a viral antigen, a bacterial antigen, a fungal antigen, a protozoa antigen, and/or a parasite antigen.

The antigens discussed herein are merely included by way of example. The list is not intended to be exclusive and further examples will be readily apparent to those of skill in the art.

As used herein the phrase “tumor antigen” refers to an antigen that is common to specific hyperproliferative disorders such as cancer. Tumor antigens are proteins that are produced by tumor cells that elicit an immune response, particularly T-cell mediated immune responses. The selection of the antigen binding moiety of the invention will depend on the particular type of cancer to be treated

According to one embodiment, the tumor antigen is associated with a solid tumor.

According to one embodiment, the tumor antigen is associated with a hematologic malignancy

The type of tumor antigen referred to in the invention includes a tumor-specific antigen (TSA) or a tumor-associated antigen (TAA). A “TSA” refers to a protein or polypeptide antigen unique to tumor cells and which does not occur on other cells in the body. A “TAA” refers to a protein or polypeptide antigen that is expressed by a tumor cell. For example, a TAA may be one or more surface proteins or polypeptides, nuclear proteins or glycoproteins, or fragments thereof, of a tumor cell.

Non-limiting examples of TSA or TAA antigens include the following: Differentiation antigens such as MART-1/MelanA (MART-1), gp 100 (Pmel 17), tyrosinase, TRP-1. TRP-2 and tumor-specific multilineage antigens such as MAGE-1, MAGE-3, BAGE, GAGE-1, GAGE-2, p15: overexpressed embryonic antigens such as CEA; overexpressed oncogenes and mutated tumor-suppressor genes such as p53. Ras, HER2/neu: unique tumor antigens resulting from chromosomal translocations; such as BCR-ABL, E2A-PRL, H4-RET, IGH-IGK. MYL-RAR; and viral antigens, such as the Epstein Barr virus antigens EBVA and the human papillomavirus (HPV) antigens E6 and E7. Other large, protein-based antigens include TSP-180, MAGE-4. MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F. 5T4, 791Tgp72, alpha-fetoprotein, beta-HCG. BCA225, BTAA. CA 125, CA 15-3\CA 27.291\BCAA, CA 195, CA 242, CA-50, CAM43, CD68\P1, CO-029, FGF-5, G250, Ga733\EpCAM, HTgp-175, M344, MA-50, MG7-Ag, MOV18, MUCI, NB/70K, NY-CO-1. NKG2DL. NR, ROBO1, RCASI, SDCCAG16, TA-90\Mac-2 binding protein\cyclophilin C-associated protein. TAAL6, TAG72, TLP, and TPS.

Further examples of tumor antigens include, but are not limited to, A33, BAGE, Bcl-2, β-catenin, BCMA, CA125, CA19-9, CD5, CD7, CD19, CD20, CD21, CD22, CD33/IL3Ra, CD34. CD37, CD38. CD45, CD123. CD135 (FLT3), CD138, carcinoembryonic antigen (CEA), CLLI, c-Met. CS-1, cyclin BI, DAGE, EBNA, EGFR. EGFRVIII, ephrinB2, estrogen receptor, FAP, ferritin, folate-binding protein, GAGE, G250, GD-2, GM2, gp75, gp 100 (Pmel 17), Glycolipid F77. HER2/neu, HPV E6, HPV E7. Ki-67, LRP, mesothelin. MY-ESO-1, MART-1, MAGE A3, p53, PRAME, PRI. PSMA, ROR1, SLAMF7, WTI (willms tumor), and the like. Further tumor antigens are provided in van der Bruggen P, Stroobant V, Vigneron N, Van den Eynde B. Peptide database: T cell-defined tumor antigens. Cancer Immun (2013), www (dot) cancerimmunity (dot) org/peptide/, incorporated herein by reference.

Additional CAR/tg-TCR targets for solid tumors are described in Ma et al., Int. J. Biol. Sci. (2019) 15 (12): 2548-2560, incorporated herein by reference.

According to a specific embodiment, the target antigen is HER2.

According to a specific embodiment, the target antigen is CD38.

According to some embodiments of the invention, the viral antigen may be derived from any virus, such as but not limited to, human immunodeficiency virus (HIV), influenza, Cytomegalovirus (CMV), T-cell leukemia virus type 1 (TAX), hepatitis C virus (HCV). (HBV), Epstein-Barr virus (EBV), Adenovirus (Adv), cold viruses, flu viruses, hepatitis A. B. and C viruses, herpes simplex, Japanese encephalitis, measles, polio, rabies, respiratory syncytial, rubella, smallpox, varicella zoster, rotavirus, West Nile virus, Polyomavirus (e.g. BK virus), severe acute respiratory syndrome (SARS) e.g. severe acute respiratory syndrome coronavirus 2 (SARS-COV-2), and/or zika virus.

According to some embodiments of the invention, the viral antigens include, but are not limited to, viral epitopes from a polypeptide selected from the group consisting of: human T cell lymphotropic virus type 1 (HTLV-1) transcription factor (TAX), influenza matrix protein epitope. Epstein-Bar virus (EBV)-derived epitope, HIV-1 RT, HIV Gag. HIV Pol, influenza membrane protein M1, influenza hemagglutinin, influenza neuraminidase, influenza nucleoprotein, influenza nucleoprotein, influenza matrix protein (M1), influenza ion channel (M2), influenza non-structural protein NS-1, influenza non-structural protein NS-2, influenza PA, influenza PB1, influenza PB2, influenza BM2 protein, influenza NB protein, influenza nucleocapsid protein, Cytomegalovirus (CMV)phosphorylated matrix protein (pp65), TAX, hepatitis C virus (HCV), HBV pre-S protein 85-66, HTLV-1 tax 11-19, HBV surface antigen 185-194, Severe acute respiratory syndrome (SARS-COV) protein S1. SARS-COV protein RBD, SARS-COV Nuclecapsid protein, SARS-COV protein Plpro. Severe acute respiratory syndrome coronavirus 2 (SARS-COV-2) protein S1. SARS-COV-2 protein S2, SARS-COV-2 protein S1+S2 ECD, SARS-COV-2 protein RBD, SARS-COV-2 protein N antigen, SARS-CoV-2 protein S antigen or SARS-COV-2 nuclecapsid protein.

According to some embodiments of the invention, the bacterial antigen may be derived from any bacteria, such as but not limited to, anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus and tetanus.

According to some embodiments of the invention, the bacterial antigens include, but are not limited to, anthrax antigens include, but are not limited to, anthrax protective antigen: gram-negative bacilli antigens include, but are not limited to, lipopolysaccharides: haemophilus influenza antigens include, but are not limited to, capsular polysaccharides: diptheria antigens include, but are not limited to, diptheria toxin: Mycobacterium tuberculosis antigens include, but are not limited to, mycolic acid, heat shock protein 65 (HSP65), the 30 kDa major secreted protein and antigen 85A: pertussis toxin antigens include, but are not limited to, hemagglutinin, pertactin. FIM2. FIM3 and adenylate cyclase: pneumococcal antigens include, but are not limited to, pneumolysin and pneumococcal capsular polysaccharides: rickettsiae antigens include, but are not limited to, rompA: streptococcal antigens include, but are not limited to, M proteins; and tetanus antigens include, but are not limited to, tetanus toxin.

According to some embodiments of the invention, the antigen is a superbug antigen (e.g. multi-drug resistant bacteria). Examples of superbugs include, but are not limited to, Enterococcus faecium. Clostridium difficile. Acinetobacter baumannii. Pseudomonas aeruginosa, and Enterobacteriaceae (including Escherichia coli, Klebsiella pneumoniae, Enterobacter spp.).

According to some embodiments of the invention, the fungal antigen may be derived from any fungi, such as but not limited to, candida, coccidiodes, cryptococcus, histoplasma, leishmania, plasmodium, protozoa, parasites, schistosomae, tinea, toxoplasma, and Trypanosoma cruzi.

According to some embodiments of the invention, the fungal antigens include, but are not limited to, coccidiodes antigens include, but are not limited to, spherule antigens: cryptococcal antigens include, but are not limited to, capsular polysaccharides: histoplasma antigens include, but are not limited to, heat shock protein 60 (HSP60); leishmania antigens include, but are not limited to, gp63 and lipophosphoglycan: Plasmodium falciparum antigens include, but are not limited to, merozoite surface antigens, sporozoite surface antigens, circumsporozoite antigens, gametocyte/gamete surface antigens, protozoal and other parasitic antigens including the blood-stage antigen pf 155/RESA: schistosomae antigens include, but are not limited to, glutathione-S-transferase and paramyosin: tinea fungal antigens include, but are not limited to, trichophytin: toxoplasma antigens include, but are not limited to. SAG-1 and p30; and Trypanosoma cruzi antigens include, but are not limited to the 75-77 kDa antigen and the 56 kDa antigen.

Various methods can be used to introduce nucleic acids of some embodiments of the invention into NK cells (e.g., nucleic acids encoding a membrane bound protein). Such methods are generally described in Sambrook et al., Molecular Cloning: A Laboratory Manual. Cold Springs Harbor Laboratory, New York (1989, 1992), in Ausubel et al., Current Protocols in Molecular Biology. John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995). Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5.464.764 and 5.487.992 for positive-negative selection methods.

According to one example, nucleic acids of some embodiments of the invention are introduced into NK cells as naked DNA or in a suitable vector. Methods of stably transfecting cells by electroporation using naked DNA are known in the art. See. e.g., U.S. Pat. No. 6,410,319. Naked DNA generally refers to the DNA encoding a membrane bound protein contained in a plasmid expression vector in proper orientation for expression.

Alternatively, a viral vector (e.g., a retroviral vector, adenoviral vector, adeno-associated viral vector, or lentiviral vector) can be used to introduce the nucleic acids of some embodiments of the invention into NK cells (e.g. nucleic acids encoding a membrane bound protein). Suitable vectors for use in accordance with the method of the present disclosure are non-replicating in the NK cells. A large number of vectors are known that are based on viruses, where the copy number of the virus maintained in the cell is low enough to maintain the viability of the cell, such as, for example, vectors based on HIV. SV40, EBV. HSV, or BPV.

According to one example, nucleic acids of some embodiments of the invention are introduced into NK cells by non-viral gene transfer.

According to one example, nucleic acids of some embodiments of the invention are introduced into NK cells as mRNA.

According to a specific embodiment, upregulating the expression of a membrane bound protein is affected by electroporation of nucleic acids (e.g., mRNA) into the NK cells. Electroporation may be affected using any electroporation device, such as but not limited to, a Nucleofector or BTX-Gemini Twin Wave Electroporator.

According to one embodiment, two, three or more membrane bound proteins may be co-expressed on a single NK cell.

According to one embodiment, the NK cell may be modified to co-express:
(i) a CAR or a tg-TCR, and
(ii) a cytokine or a receptor which effects the survival of the NK cells in vivo (e.g. a IL-15, IL-15R, Receptor Linker IL-15 (RLI), TLR, etc.)

According to a specific embodiment, when the gene of interest is CISH, the at least one membrane bound protein comprises IL-15.

According to a specific embodiment, when the gene of interest is CD38, the at least one membrane bound protein comprises anti-CD38 CAR.

According to one embodiment, upregulating the expression of a membrane bound protein is affected 8-20 days, 8-18 days, 10-18 days, 12-18 days, 12-16 days, 12-14 days from initiation of the cell culture.

According to a specific embodiment, upregulating the expression of a membrane bound protein is affected 12-16 days from initiation of the cell culture.

According to a specific embodiment, upregulating the expression of a membrane bound protein is affected 12-14 days from initiation of the cell culture.

In certain embodiments, after the NK cells have been modified to express at least one membrane bound protein, the cells may be harvested from the culture.

According to a specific embodiment, the cells are modified to express at least one membrane bound protein 1-4 days, 1-3 days. 1-2 days or 0.5-1 day prior to harvesting of the cells.

Harvesting of the cells can be performed manually, by releasing attached cells (e.g. “scraping” culture vessel surfaces) or by a cell harvesting device, which is designed to efficiently wash cells out of their culture vessels and collect the cells automatically. In specific embodiments, the expanded NK cells are harvested from the culture vessels by a cell harvesting device (e.g., the harvesting device of the G-Rex MCS, WolfWilson, St Paul MN). In specific embodiments, the expanded CD3− depleted NK cell fraction is harvested from the culture vessels by a cell harvesting device (e.g., the LOVO Cell Processing device by Fresenius Kabi (Hamburg, Germany)).

In some embodiments, harvesting of expanded NK cells from culture removes most, or nearly all of the cells from the culture vessel. In other embodiments, harvesting can be performed in two or more steps, allowing the unharvested cells to remain in culture until harvested at a later time. In certain embodiments, the expanded NK cells are harvested in two steps, comprising harvesting a first portion of the expanded NK cells, and then harvesting a second portion of the expanded NK cells. Harvesting the two portions can be performed with an interval of hours, days or more between harvesting of the first and second portion. The two portions harvested can comprise approximately equal portions of the culture (e.g., equal amounts of the cultured NK cells), or one of the portions may be comprise a larger fraction of the cultured NK cells than the other). According to one embodiment, harvesting comprises harvesting the expanded modified NK cells about 12-18 days, e.g., 14-16 days, following initiation of culture. According to one embodiment, harvesting comprises harvesting the expanded modified NK cells about 1-4 days, e.g., 1-2 days, after modifying the cells to express at least one membrane bound protein (e.g. CAR).

In order to prepare the expanded population of NK cells for use, the harvested cells need to be washed of culture medium, critical parameters evaluated and volume adjusted to a concentration suitable for infusion over a clinically reasonable period of time.

Following harvesting, the expanded modified NK cells can be washed free of culture medium manually or, preferably for clinical applications, using an automated device employing a closed system. Washed cells can be reconstituted with an infusion solution (for example, one exemplary infusion solution comprises 8% w/v HSA and 6.8% w/v Dextran-40). In some embodiments, the reconstitution is performed in a closed system. In some embodiments, the infusion solution is screened for suitability for use with the methods and compositions of the present invention. Exemplary criteria for selection of suitable infusion solution include safety tests indicating no bacterial, yeast or mold growth, endotoxin content of less than 0.5 Eu/ml and a clear, foreign particle-free appearance.

Once the expanded modified NK cells are obtained, the cells are examined for the number of cells (i.e. proliferation), for cell signature (e g. CD3−CD56+ cells), for the expression of the membrane bound protein (e.g. CAR, tg-TCR, IL-15, RLI, etc.) and for NK cell functionality.

Assays for cell proliferation are well known in the art, and include without being limited to, clonogenic assays, in which cells are seeded and grown in low densities, and colonies counted, mechanical assays [flow cytometry (e.g., F ACS™), propidium iodide], which mechanically measure the number of cells, metabolic assays (such as incorporation of tetrazolium salts e.g., XTT. MTT, etc.), which measure numbers of viable cells, direct proliferation assays (such as bromodeoxyuridine, thymidine incorporation, and the like), which measure DNA synthesis of growing populations.

Assays for cell signature and for expression of proteins on a cell membrane are well known in the art, and include without being limited to. FACS analysis and immunohistological staining techniques.

As used herein, the term “NK cell functionality” refers to any biological function ascribed to NK cells. A non-limiting list of NK cell functions includes, for example, cytotoxicity, induction of apoptosis, cell motility, directed migration, cytokine and other cell signal response, cytokine/chemokine production and secretion, expression of activating and inhibitory cell surface molecules in-vitro, cell homing and engraftment (in vivo retention) in a transplanted host, and alteration of disease or disease processes in vivo. In some embodiments, NK cell functions enhanced by expansion in the presence of nicotinamide and/or other nicotinamide moiety include at least one of elevated expression of CD62L surface marker, elevated migration response, and greater cytotoxic activity of the NK cells, as well as elevated homing and in vivo retention of infused NK cells.

Assays for adhesion and migration molecules such as CD62L, CXCR-4, CD49e and the like, important for homing/engraftment and retention of cells in transplantation, are well known in the art. CD62L expression in a cell can be assayed, for example, by flow cytometry, immunodetection, quantitative cDNA amplification, hybridization and the like.

Assays for cells migration are well known in the art. Migration of cells can be assayed, for example, by transmigration assays or gap closure assays. In one embodiment, migration potential of different populations of NK cells is determined by the “Transwell” IM transmigration assay.

Assays for cytotoxicity (“cell killing”) are well known in the art. Examples of suitable target cells for use in redirected killing assays are cancer cell line, primary cancer cells solid tumor cells, leukemic cells, or virally infected cells. Particularly. K562, BL-2, colo250 and primary leukaemic cells can be used, but any of a number of other cell types can be used and are well known in the art (see, e.g., Sivori et al. (1997) J. Exp. Med. 186:1129-1136; Vitale et al. (1998) J. Exp. Med. 187:2065-2072; Pessino et al. (1998) J. Exp. Med. 188:953-960; Neri et al. (2001) Clin. Diag. Lab. Immun. 8:1131-1135). For example, cell killing may be assessed by cell viability assays (e.g., dye exclusion, chromium release, CFSE), metabolic assays (e.g., tetrazolium salts), and direct observation.

The washed and concentrated expanded modified NK cell fraction generated by some embodiments of the invention is characterized by comprising about 60% to about 99% CD56+/CD3− cells, about 70% to about 99% CD56+/CD3− cells, about 80% to about 99% CD56+/CD3− cells or about 90-99% CD56+/CD3− cells. In one embodiment, the washed and concentrated expanded NK cell fraction generated by some embodiments of the invention is characterized by comprising at least about 60%, at least 70%, at least 80%, at least 90%, or at least 95% CD56+/CD3− cells.

The washed and concentrated expanded modified NK cell fraction generated by some embodiments of the invention is characterized by comprising about 60% to about 99% membrane bound protein positive cells, about 70% to about 99% membrane bound protein positive cells, about 80% to about 99% membrane bound protein positive cells or about 90-99% membrane bound protein positive cells (e.g., CAR, tg-TCR, IL-15, RLI, etc.). In one embodiment, the washed and concentrated expanded NK cell fraction generated by some embodiments of the invention is characterized by comprising at least about 60%, at least 70%, at least 80%, at least 90%, or at least 95% membrane bound protein positive cells (e.g., CAR, tg-TCR. IL-15, RLI, etc.).

The modified NK cells of some embodiments of the invention may be used as fresh cells. Alternatively, the cells may be cryopreserved for future use, or “off the shelf” use.

According to an aspect of some embodiments of the invention there is provided an isolated population of NK cells obtainable according to the methods of some embodiments of the invention.

According to one embodiment, the isolated population of NK cells (i.e. following ex vivo expansion, e.g. at the end of culture) comprise at least about 50%, 60%, 70%, 75%, 80%, 85%, 90% or 95% or more NK cells.

According to one embodiment, at least about 50%, 60%, 70%, 75%, 80%, 85%, 90% or 95% or more of the isolated population of NK cells (i.e. following ex vivo expansion. e.g. at the end of culture) are genetically modified.

According to one embodiment, at least about 50%, 60%, 70%, 75%, 80%, 85%, 90% or 95% or more of the isolated population of NK cells (i.e. following ex vivo expansion, e.g. at the end of culture) comprise an upregulated expression of at least one membrane bound protein.

According to one embodiment, at least about 50%, 60%, 70%, 75%, 80%, 85%, 90% or 95% or more of the isolated population of NK cells (i.e. following ex vivo expansion. e.g. at the end of culture) are both genetically modified and comprise an upregulated expression of at least one membrane bound protein.

The isolated population of NK cells of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the isolated population of NK cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences”, Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion): molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB, pharmacological strategies designed to increase the lipid solubility of an agent (e.g, conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method. Ultimately, one may administer the pharmaceutical composition in a local rather than systemic manner, for example, via injection of the pharmaceutical composition directly into a tissue region of a patient.

According to one embodiment, the route of administration includes, for example, an injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the pharmaceutical composition of the present invention is administered to a patient by intradermal or subcutaneous injection. In another embodiment, the pharmaceutical composition of the present invention is preferably administered by i.v. injection. The pharmaceutical composition may be injected directly into a tumor, lymph node, or site of infection.

Pharmaceutical compositions of some embodiments of the invention may be manufactured by processes well known in the art, e.g., by means of conventional mixing, dissolving, granulating, dragée-making, levitating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art

For oral administration, the pharmaceutical composition can be formulated readily by combining the active compounds with pharmaceutically acceptable carriers well known in the art. Such carriers enable the pharmaceutical composition to be formulated as tablets, pills, dragées, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatin, tragacanth, methyl cellulose, hydroxypropylmethyl-cellulose, sodium gum carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragée cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragée coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of. e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion.

Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes.

Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of some embodiments of the invention may also be formulated in rectal compositions such as suppositories or retention enemas, using. e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (isolated population of NK cells) effective to prevent, alleviate or ameliorate symptoms of a disorder (e.g., malignant or non-malignant disease) or prolong the survival of the subject being treated.

Determination of a therapeutically effective amount is well within the capability of those skilled in the art, especially in light of the detailed disclosure provided herein.

When “therapeutic amount” is indicated, the precise amount of the compositions of the present invention to be administered can be determined by a physician with consideration of individual differences in age, weight, disease state, e.g. tumor size, extent of infection or metastasis, and the condition of the patient (subject). It can generally be stated that a pharmaceutical composition comprising the cells described herein may be administered at a dosage of 25-500×10⁶ cells per kg body weight, e.g. 25-400×10⁶ cells per kg body weight, 50-300×10⁶ cells per kg body weight, e.g. 50-250×10⁰ cells per kg body weight, including all integer values within those ranges. According to one embodiment, the cells described herein may be administered at a dosage of about 25×10⁶ cells per kg body weight, about 50×10⁶ cells per kg body weight, about 75×10⁶ cells per kg body weight, about 100×10⁶ cells per kg body weight, about 150×10⁶ cells per kg body weight, about 200×10⁶ cells per kg body weight, about 250×10⁶ cells per kg body weight, or about 300×10⁶ cells per kg body weight.

The NK cell compositions of some embodiments of the invention may also be administered multiple times at these dosages The NK cells can be administered by using infusion techniques that are commonly known in immunotherapy (see. e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly

For example, the effect of the active ingredients (e.g., the isolated population of NK cells of some embodiments of the invention) on the pathology can be evaluated by monitoring the level of cellular markers, hormones, glucose, peptides, carbohydrates, cytokines, etc. in a biological sample of the treated subject using well known methods (e.g. ELISA, FACS, etc) or by monitoring the tumor size using well known methods (e.g. ultrasound, CT, MRI, etc).

For any preparation used in the methods of the invention, the therapeutically effective amount or dose can be estimated initially from in vitro and cell culture assays For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”. Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved. According to one embodiment, the dosing can be one, two, three or more administrations per day. The dosing can be on subsequent days, or within days or weeks apart. Such determinations can readily be determined by one skilled in the art of medicine.

The amount of a composition to be administered will, of course, be dependent on the subject being treated, the severity of the affliction, the manner of administration, the judgment of the prescribing physician, etc.

According to some embodiments of the invention, the therapeutic agent of the invention can be provided to the subject in conjunction with other drug(s) designed for treating the pathology [i.e. combination therapy, e.g., before, concomitantly with, or following administration of the isolated population of NK cells].

According to one embodiment of the invention, the isolated population of NK cells of some embodiments of the invention may be used in combination with chemotherapy, radiation therapy, immunosuppressive agents (e.g. cyclosporin, azathioprine, methotrexate, mycophenolate, and FK506), antibodies, or other agents known in the art.

In certain embodiments, the isolated population of NK cells of some embodiments of the invention are administered to a patient in conjunction with any number of relevant treatment modalities, including but not limited to treatment with agents such as antiviral agents (e.g. Ganciclovir, Valaciclovir, Acyclovir, Valganciclovir, Foscarnet, Cidofovir. Maribavir, Leflunomide), chemotherapeutic agents (e.g. antineoplastic agents, such as but not limited to, Alkylating agents including e.g. Cyclophosphamide, Busulfan, Mechlorethamine or mustine (HN2), Uramustine or uracil mustard, Melphalan. Chlorambucil, Ifosfamide, Bendamustine, Nitrosoureas Carmustine, Lomustine. Streptozocin, Thiotepa, Cisplatin, Carboplatin, Nedaplatin, Oxaliplatin, Satraplatin, Triplatin tetranitrate, Procarbazine, Altretamine, Triazenes (dacarbazine, mitozolomide, temozolomide), Dacarbazine. Temozolomide, Myleran, Busulfex. Fludarabine, Dimethyl mileran or Cytarabine) or therapeutic monoclonal antibodies (e.g. Trastuzumab (Herceptin R), Pertuzumab (PerjetaR), Certuximab (Erbitux®), Panitumumab (Vectibix R), Necitumumab (Portrazza®), Dinutuximab (Unituxin®), Bevacizumab (Avastin R), Ramucirumab (Cyramza R), Olaratumab (Lartruvo R), Ipilimumab (Yervoy®). Nivolumab (Opdivo®). Pembrolizumab (Keytruda®), Atezolizumab (TecentriqR), Ado-trastuzumab emtansine (KadcyclaR) fusion, Denosumab (XgevaR), Alemtuzumab (Campath®), Avelumab (Bavencio®), Blinatumomab (Blincyto®), Brentuximab vedotin (Adcetris R). Capromab pendetide (ProstaScintR), Daratumumab (Darzalex®), Durvalumab (ImfinziR), Elotuzumab (EmplicitiR), Ibritumomab tiuxetan (Zevalin®), Obinutuzumab (GazyvaR), Ofatumumab (ArzerraR), Pertuzumab (PerjetaR), Rituximab (Rituxan R), Rituximab-hyaluronidase (Rituxan Hycela R), Inotuzumab ozogamicin (Besponsa R). Bevacizumab-awwb (Mvasi &), Trastuzumab dkst (Ogivri®), or Tositumomab (Bexxar®)).

In a specific embodiment, the isolated population of NK cells of some embodiments of the invention are administered to a patient in conjunction with Daratumumab (DARA).

In a specific embodiment, the isolated population of NK cells of some embodiments of the invention are administered to a patient in conjunction with Rituximab.

It will be appreciated that the isolated population of NK cells of some embodiments of the invention may be administered to a patient in conjunction with a chemotherapeutic agent, radiation therapy, antibody therapy, surgery, phototherapy, etc.

The combination therapy may increase the therapeutic effect of the agent of the invention in the treated subject.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U. S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The kit may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to an aspect of some embodiments of the invention, there is provided a method of treating a disease in a subject in need thereof, the method comprising administering to the subject a therapeutically effective amount of the isolated population NK cells of some embodiments of the invention, thereby treating the subject.

According to an aspect of some embodiments of the invention, there is provided a therapeutically effective amount of the isolated population of NK cells of some embodiments of the invention for use in treating a disease in a subject in need thereof.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” or “subject in need thereof” refers to a mammal, preferably a human being, male or female at any age or gender that suffers from a disease which may be treated with the NK cells.

Thus, the method of the present invention may be applied to treat any disease such as, but not limited to, a malignant disease (e.g. cancer) and an infectious disease (e.g. viral infection, bacterial infection, fungal infection, protozoan infection or parasitic infections).

According to one embodiment, the subject has a malignant disease.

### Cancerous Diseases

Malignant diseases (also termed cancers) which can be treated by the method of some embodiments of the invention can be any solid or non-solid tumor and/or tumor metastasis.

Examples of cancer include, but are not limited to, carcinoma, lymphoma, blastoma, sarcoma, and leukemia. More particular examples of such cancers include squamous cell cancer, soft-tissue sarcoma, Kaposi's sarcoma, melanoma, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, rectal cancer, endometrial or uterus cancer e.g. uterine carcinoma, carcinoid carcinoma, salivary gland carcinoma, kidney or renal cancer, liver cancer, prostate cancer, vulval cancer, thyroid cancer, hepatic carcinoma, mesothelioma, a myeloma e.g. multiple myeloma, post-transplant lymphoproliferative disorder (PTLD), neuroblastoma, esophageal cancer, synovial cell cancer, glioma and various types of head and neck cancer (e.g. brain tumor). The cancerous conditions amenable for treatment of the invention include metastatic cancers.

According to one embodiment, the malignant disease is a hematological malignancy. Exemplary hematological malignancies include, but are not limited to, leukemia [e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia, T-cell acute lymphocytic leukemia (T-ALL) and B-cell chronic lymphocytic leukemia (B-CLL)] and lymphoma [e.g., Hodgkin's disease, non-Hodgkin's lymphoma. Burkitt, cutaneous T cell, histiocytic, lymphoblastic, T cell, thymic, B cell, including low grade/follicular: small lymphocytic (SL) NHL; intermediate grade/follicular NHL; intermediate grade diffuse NHL: high grade immunoblastic NHL: high grade lymphoblastic NHL; high-grade small non-cleaved cell NHL; bulky disease NHL: mantle cell lymphoma: AIDS-related lymphoma; and Waldenstrom's Macroglobulinemia].

According to some embodiments of the invention, the pathology is a solid tumor. According to some embodiments of the invention, the pathology is a tumor metastasis.

According to some embodiments of the invention, the pathology is a hematological malignancy

According to a specific embodiment, the malignant disease is leukemia or a lymphoma.

According to a specific embodiment, the malignant disease is a multiple myeloma.

### Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Specific types of viral pathogens causing infectious diseases treatable according to the teachings of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to the teachings of the present invention include, but are not limited to, those caused by human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection. COVID-19 infection. Herpes simplex virus infection, yellow fever. Ebola virus infection, rabies, Adenovirus (Adv), cold viruses, flu viruses, Japanese encephalitis, polio, respiratory syncytial, rubella, smallpox, varicella zoster, rotavirus, West Nile virus and zika virus.

According to a specific embodiment, the viral disease is caused by a virus selected from the group consisting of Epstein-Barr virus (EBV), cytomegalovirus (CMV), BK Virus, Adenovirus (Adv), severe acute respiratory syndrome (SARS), severe acute respiratory syndrome coronavirus 2 (SARS-COV-2), immunodeficiency virus (HIV), influenza. Cytomegalovirus (CMV), T-cell leukemia virus type 1 (TAX), hepatitis C virus (HCV) or hepatitis B virus (HBV).

Specific examples of bacterial infections which may be treated according to the teachings of the present invention include, but are not limited to, those caused by anthrax: gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus and tetanus.

Specific examples of superbug infections (e.g. multi-drug resistant bacteria) which may be treated according to the teachings of the present invention include, but are not limited to, those caused by Enterococcus faecium, Clostridium difficile, Acinetobacter baumannii, Pseudomonas aeruginosa, and Enterobacteriaceae (including Escherichia coli, Klebsiella pneumoniae. Enterobacter spp.).

Specific examples of fungal infections which may be treated according to the teachings of the present invention include, but are not limited to, those caused by candida, coccidiodes, cryptococcus, histoplasma, leishmania, plasmodium, protozoa, parasites, schistosomae, tinea, toxoplasma, and Trypanosoma cruzi.

Clinical experience with NK cell therapy has shown that allogeneic NK cells can successfully engraft in hosts, with a lower incidence of graft versus host disease (GVHD). When the identity of the candidate for transplantation (e.g., the “subject”) is known, parameters such as HLA-match (compatibility) can be determined and serve as a selection criteria. According to one embodiments, the NK cells are selected from an HLA-haploidentical or HLA-mismatched donor. The NK cell donor can be related, or non-related donor. According to one embodiments, the NK cells are obtained from a syngeneic donor.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” O a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example. “Molecular Cloning: A laboratory Manual” Sambrook et al., (1989): “Current Protocols in Molecular Biology” Volumes I-III Ausubel, R. M., ed. (1994): Ausubel et al., “Current Protocols in Molecular Biology”, John Wiley and Sons. Baltimore, Maryland (1989): Perbal, “A Practical Guide to Molecular Cloning”, John Wiley & Sons, New York (1988): Watson et al., “Recombinant DNA”. Scientific American Books, New York: Birren et al. (eds) “Genome Analysis: A Laboratory Manual Series”, Vols. 1-4, Cold Spring Harbor Laboratory Press, New York (1998): methodologies as set forth in U.S. Pat. Nos. 4,666.828; 4,683,202; 4.801,531:5, 192,659 and 5,272.057; “Cell Biology: A Laboratory Handbook”, Volumes I-III Cellis. J. E., ed. (1994): “Current Protocols in Immunology” Volumes I-III Coligan J. E., ed. (1994): Stites et al. (eds), “Basic and Clinical Immunology” (8th Edition), Appleton & Lange, Norwalk, CT (1994); Mishell and Shiigi (eds), “Selected Methods in Cellular Immunology”. W. H. Freeman and Co., New York (1980); available immunoassays are extensively described in the patent and scientific literature, see, for example, U.S. Pat. Nos. 3,791,932:3,839,153:3,850,752:3, 850,578; 3.853,987; 3.867,517:3.879,262; 3.901,654; 3.935,074:3,984,533:3.996,345; 4.034,074:4,098.876; 4,879,219:5,011.771 and 5,281.521; “Oligonucleotide Synthesis” Gait. M. J., ed. (1984); “Nucleic Acid Hybridization” Hames, B. D., and Higgins S. J., I eds. (1985); “Transcription and Translation” Hames, B. D., and Higgins S. J., Eds. (1984); “Animal Cell Culture” Freshney. R. I . . . ed. (1986): “Immobilized Cells and Enzymes” IRL Press, (1986): “A Practical Guide to Molecular Cloning” Perbal, B., (1984) and “Methods in Enzymology” Vol. 1-317, Academic Press, “PCR Protocols: A Guide To Methods And Applications”, Academic Press, San Diego, CA (1990); Marshak et al, “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Ex Vivo Cultures with T Cells as Feeder Cells

On day 0, blood cells were collected by apheresis from a healthy donor. Red blood cells (RBC) were lysed by washing with ACK buffer (Gibco, Dublin, Ireland). CD3+ cells were depleted using CliniMACS and CD3 reagent (Miltenyi Biotec, Germany) according to the manufacturer's instructions.

CD3− depleted cells were washed by CliniMACS buffer with 20% HSA and resuspended in complete MEMα media.

Cells were seeded in MEMα medium containing 0.05 mg/ml Gentamicin (Braun), 2 mM L-glutamine (HyClone), and further supplemented with 10% human AB serum (Gemini), 7 mM nicotinamide (Vertillus) and 20 ng/ml IL-15 (Miltenyi).

0.35×10⁶ cells/ml of CD3− depleted cells were seeded in a GREX100MCS cell culture flask (Wilson Wolf) containing 400 mL MEMα medium and further comprising irradiated CD3+ cells as feeder cells (i.e. irradiated at 40 Gy. 130 KV, 5 mA) at a ratio of 1:1, and 10 ng/ml OKT-3 (Miltenyi). Cells were incubated at 5% CO₂ and 37° C., humidified incubator.

On day 6-9, 400 mL of MEM˜ medium was added to each G-REX100MCS culture flasks to double the volume.

On day 12-14, cells were counted and prepared for mRNA electroporation for transient expression a membrane bound protein of interest, such as a chimeric antigen receptor (CAR), IL-15, etc., as described below. After electroporation, cells were transferred to a 24-well plate with human-serum-enriched MEMα medium as described above. Cells were recovered for 24-48 hours and then analyzed.

(Optionally, the CD3− depleted cells are further prepared for electroporation for knockout of gene expression as described below at 24-72 hours from the start of culture. After electroporation, CD3− depleted cells are seeded in a GREX100MCS cell culture flask for the duration of 12-14 days of culture, as discussed above).

Ex Vivo Cultures with PBMCs as Feeder Cells

On day 0, blood cells were collected by apheresis from a healthy donor. Red blood cells (RBC) were lysed by washing with ACK buffer (Gibco, Dublin, Ireland). CD56+ cells were positively selected using CD56 MicroBeads and LS Column, according manufacturer's instructions (Miltenyi Biotec: Cat. No. 130-050-401 and Cat. No. 130-042-401, respectively). Alternatively, CD56+ cells are selected by negative selection using a mix of MicroBeads (Miltenyi (

CD56+ cells were washed by CliniMACS buffer with 20% HSA resuspended in medium supplemented with 10% human serum and 50 ng/ml IL-2, and seeded in flasks in a concentration of 2×10⁶ cells/ml.

After 24-72 hours, cells were harvested, counted, tested for viability using CEDEX counting machine and flow cytometry viability dye and prepared for electroporation for knockout of gene expression as described below.

After electroporation, cells were seeded in MEMI medium containing 0.05 mg/ml Gentamicin (Braun), 2 mM L-glutamine (HyClone), and further supplemented with 10% human AB serum (Gemini). 7 mM nicotinamide (Vertillus) and 20 ng/ml IL-15 (Miltenyi).

4×10⁶ cells/ml of CD56+ cells were seeded in a 6-well Grex culture flask (Wilson Wolf) containing 16 mL MEMα medium and further comprising irradiated peripheral blood mononuclear cells (PBMCs) (fresh or thawed) as feeder cells (i.e. irradiated at 40 Gy, 130 KV. 5 mA) at a ratio of 1:1, and 10 ng/ml OKT-3 (Miltenyi). Cells were incubated at 5% CO₂ and 37° C., humidified incubator.

On day 6-9, 16 mL of MEMα medium was added to each 6-well Grex culture flasks to double the volume.

On day 14-16, cells were counted and analyzed.

Optionally, on day 12-14 the cells were further prepared for mRNA electroporation for transient expression of a membrane bound protein of interest (e.g. CAR, IL-15etc.) as described below. After electroporation, cells are transferred to a 24-well plate with human-serum-enriched MEMα medium as described above. Cells are recovered for 24-48 hours and then analyzed).

mRNA Electroporation for Transient Protein Expression

At days 12-14 of culture, cells were counted, washed with PBSx1, and then washed again with cold Opti-MEM™ (Gibco™).

For mRNA electroporation, 2×10⁶-4×10⁶ cells, and 10-30 μg mRNA at a final volume of 100 μl, were used. The electroporation was performed in 2 mm cold cuvette in a maximum volume of 400 μl (scaling up per the amounts above), using BTX-Gemini Twin Wave Electroporator at a calibrated program (at voltage 300, duration 1 msc, I pulse of square wave).

TABLE 1
list of sequences for mRNA expression

| Protein | SEQ ID NO: |
|---|---|
| Receptor Linker IL-15 (RLI) | 23-25 |
| GDA -301A | |
| Receptor Linker IL-15 (RLI) | 26-28 |
| GDA-301B | |
| anti-p38 CAR | 35-37 |
| GDA-601 | |
| anti-Her2 CAR | 60-61 |
| GDA-501.A | |
| anti-Her2 CAR | 62-63 |
| GDA-501.B | |
| anti-Her2 CAR | 64-65 |
| GDA-501.C | |
| anti-Her2 CAR | 66-67 |
| GDA-501.D | |

Following the electroporation, cells were transferred to 12-well plate with human-serum-enriched MEME medium as described above. Cells were recovered for 24 hours and then analyzed.

### Gene Knockout

Screening for gRNAs

The DNA sequence of each guide RNA was cloned into CRIS PR expression plasmid and genome editing experiments were performed in the Hek293 cell line. The most active gRNAs (see Table 2, III below) were chosen for further experiments. To evaluate Editing efficiency, the manipulated loci were amplified by PCR with suitable primers (see Table 3, below) and sequenced by SANGER Sequencing. INDEL editing percentages E analyzed by TIDE.

TABLE 2
gRNA Sequences for CD38 and CISH

| | gRNA Sequence | SEQ ID NO: |
|---|---|---|
| CD38-G5 | TGTAGACTGCCAAAGTGTAT | 1 |
| CISH 1 | GGGTTCCATTACGGCCAGCG | 4 |
| CISH-G4 | GTCCTTTGCTGGCTGTGGAG | 5 |
| (CISH2) | | |
| CISH-G10 | GTTGGAGTCCAGACGGAAGC | 6 |
| (CISH3) | | |

TABLE 3
Primers Sequences

| | Forward | Reverse |
|---|---|---|
| CD38 | 5′CCTGGTAGACTGC | 5′AGCCCCTTTCCTA |
| | ATGTTAGACGAG3′ | TCTCTGTATCCC3′ |
| | (SEQ ID NO: 2) | (SEQ ID NO: 3) |
| CISH | 5′CTGCTTCTGCGTA | 5′GGACTAACTGAGC |
| | CAAAGGGC3′ | CCATGGCTG3′ |
| | (SEQ ID NO: 7) | (SEQ ID NO: 8) |

### RNP Complex Preparation

Chemically modified sg RNAs oligomers were synthesized by Integrated DNA □ Technologies (IDT; Coralville, IA, USA). For each electroporation reaction, 416 pmol of □

Alt-R Cas9 protein (IDT. Coralville, IA. US A) was complexed with Alt-R sg RNAs in a 1:2.5 molar ratio (1040 pmol per reaction) in a PBS solution (20 μl total volume). Complexes were allowed to form for 10-20 min at room temperature before electroporation.

### Genome Editing by RNP Electroporation in NK Cells

At 24-72 hours of culture, CD56+NK cells were counted, washed with PBSx1, and then were resuspended in Opti-MEM™ (Gibco™). 2×10⁶-4×10⁶ cells were used in 80 μl Opti-MEM™ per reaction, and were mixed with RNP complexes at a final concentration of 4 μM. Cells were then supplemented with 4 μl (100 μM) Alt-R Enhancer (IDT. Coralville, IA, USA). The supplemented cell solution was transferred into the BTX-Gemini Twin Wave Electroporator and electroporated using a calibrated program (at voltage 300, duration 2 msc).

Following the electroporation, cells were transferred to GREX culture device and cultured (as discussed above) for 12-14 days. At the end of culture, cells were harvested and analyzed. In addition, at the same time points, genomic DNA was extracted with QuickExtract (Lucigen, Middleton, WI, USA). To evaluate Editing efficiency, the manipulated loci were amplified by PCR with suitable primers (table 2.) and sequenced by SANGER Sequencing. INDEL editing percentages analyzed by TIDE.

### FACS Analysis

For FACS analysis cells were stained with the following fluorescent antibodies:

TABLE 4
List of antibodies for FACS

| Antibody | Catalog No. |
|---|---|
| CD56 VioBright B515 (FITC) | Miltenyi 130-114-552 |
| CD340 (eRBb-2) (APC) | Miltenyi 130-124-467 |
| CD38 (APC) | Miltenyi 130-113-429 |
| Viability- Helix NP Blue | Biolegend 425305 |

### Sandwich Flow Cytometry Technique

This method was used to determined CAR expression on NK cells. The term “sandwich” was used at the method follows the following order: At the bottom—the CAR-expressing NK cells; in the middle—the protein bound by the CAR; and on the top—The AB conjugate to the epitope on the protein.

For example, to detect anti-Her2 CAR expression on NK cell surface, the NK cells were cultured with 1 μg of Her2 soluble protein for 30 minutes, washed, and then an anti-Her2 APC antibody was added and cultured with the cells for 15 minutes.

### Potency Assay (Intracellular Staining of Proinflammatory Cytokines and CD107a Degranulation Marker)

Potency assay analyzes the expression of various activation markers both intracellular and surface expressed. Selected markers were both indicators of direct cellular cytotoxicity and secretion of pro-inflammatory cytokines capable of promoting the anti-tumoral activity of NK cells.

One of the mechanisms in which the NK cells kill its target is through the release of cytotoxic molecules from lytic granules. This process involves the fusion of the granule membrane with the cytoplasmic membrane of the NK cell, resulting in surface exposure of lysosomal-associated proteins that are typically present on the lipid bilayer surrounding lytic granules, such as CD107a. Therefore, membrane expression of CD107a constitutes a marker of immune cell activation and cytotoxic degranulation. Another killing mechanism the NK cells possess is through the death receptor-induced target cell apoptosis. Activated NK cells secrete a wide variety of cytokines such as IFN-γ and TNFα, GM-CSF and more. IFN-γ is one of the most potent effector cytokines secreted by NK cells and plays a crucial role in antitumor activity. IFN-γ has been shown to modulate caspase, FasL, and TRAIL expression and activates antitumor immunity. As such the potency of the NK cells was evaluated based on the expression of CD107a, TNFα and IFN-γ.

1×10⁶ NK cells were co-cultured with 0.5×10⁶ target cells (K562, RAJI)+/−RTX (0.5 μg/ml) and 2 μl of CD107a antibody was added in a total volume of 1 ml NK medium (MEMα +10% AB serum) in a FACS tube. The controls were prepared as follows: positive control: NK cells+5 μl PMA (50 ng/ml)+1 μl lonomycin (1 μg/ml), negative control: NK cells (No target) and the size control: NK. K562. RAJI, NK+K562. NK+RAJI. The cells were centrifuged for 30 sec at 300 rpm and incubated at 37° C. for 30 minutes. After the incubation, BFA and Monensin/GolgiStop (5 μg/ml final conc' BFA, 4 μl GS) were added to each tube. The cells were centrifuged for 30 sec at 300 rpm and incubated at 37° C. for 3.5 hours after which the Zombie viability dye was added and washed.

Cells were stained first for cell surface markers as follows: 1.5 μl of the outer membrane antibody (CD56, CD 16) was added and incubated for 10 minutes in the dark in 2-8° C. and washed. The Inside Stain Kit (Miltenyi, CAT #130-090-4777) was used and added for intracellular staining at this point. Cells were fixed and permeabilized, following centrifugation intracellular mAbs were added (IFN-γ and TNF-E) and the cells were incubated for 15 min at room temperature in the dark. The cells were then washed and analyzed.

TABLE 5
List of antibodies for potency assay and degranulation

| Antibody | Fluorochrome | Vendor and Catalog No. |
|---|---|---|
| Zombie Violet | B.V 421 | Bio Legend, |
| Viability Dye | | Cat. No. 4253 |
| CD 107a | VioGreen | Miltenyi |
| | | Cat. No. 130-111-629 |
| CD56 | FITC | Miltenyi |
| | | Cat. No. 130-114-549 |
| TNF-α | PE | Miltenyi |
| | | Cat. No. 130-118-974 |
| IFN-γ | PE-Vio770 | Miltenyi |
| | | Cat. No. 130-113-499 |
| GM-CSF | APC | Miltenyi |
| | | Cat. No. 130-123-420 |
| CD16 | APC- Vio770 | Miltenyi |
| | | Cat. No. 130-113-390 |

### Cytotoxicity Assay/Killing Assay (IncuCyte)

Cytotoxic killing assay was performed via the live-cell imaging system IncuCyte S3, allowing collection of real-time data regarding NK activity. Tumor target cells were labeled with CFSE dye (Life Technologies) and co-cultured with NK cells for 20 hours in a presence of PI (propidium iodide, Sigma) in the media. Viable cells remained unstained whereas dead cells were detected by overlap of the CFSE fluorescence staining and PI.

### Exemplary Embodiments

Embodiment 1. A method of ex vivo producing genetically modified natural killer (NK) cells, the method comprising:
(a) downregulating expression of a gene of interest in a population of NK cells so as to obtain a population of NK cells having been genetically modified to down-regulate a gene of interest;
(b) expanding said population of NK cells having been genetically modified to down-regulate a gene of interest so as to obtain an ex vivo expanded population of NK cells; and
(c) upregulating expression of at least one membrane bound protein in said ex vivo expanded population of NK cells, thereby producing the genetically modified NK cells.

Embodiment 2. A method of ex vivo producing natural killer (NK) cells expressing at least one membrane bound protein, the method comprising:
(a) expanding a population of NK cells by a method comprising:
   (i) culturing said population of NK cells under conditions allowing for cell proliferation, wherein said conditions comprise providing an effective amount of nutrients, serum, IL-15 and nicotinamide; and
   (ii) supplementing said population of NK cells with an effective amount of fresh nutrients, serum, IL-15 and nicotinamide 5-10 days following step
   (i) to produce expanded NK cells;
   so as to obtain an ex vivo expanded population of NK cells, and
(b) upregulating expression of at least one membrane bound protein in said ex vivo expanded population of NK cells, thereby producing the NK cells expressing the at least one membrane bound protein.

Embodiment 3. The method of embodiment 1 or 2, wherein said population of NK cells is derived from cord blood, peripheral blood, bone marrow, CD34+ cells or iPSCs.

Embodiment 4. The method of any one of embodiments 1-3, wherein said population of NK cells is deprived of CD3+ cells.

Embodiment 5. The method of any one of embodiments 1-4, wherein said population of NK cells comprises CD3 CD56+ cells.

Embodiment 6. The method of any one of embodiments 1 or 3-5, wherein said downregulating is effected by a gene editing system.

Embodiments 7. The method of any one of embodiments 1 or 3-6, wherein said NK cells are in a culture.

Embodiment 8. The method of embodiment 7, wherein said downregulating is affected 24-72 hours from initiation of said culture.

Embodiment 9. The method of any one of embodiments 1 or 3-8, wherein said gene of interest comprises a gene whose product effects proliferation and/or survival of said NK cells.

Embodiment 10. The method of any one of embodiments 1 or 3-9, wherein said gene of interest is selected from the group consisting of CISH, TGFβ receptor and CD38.

Embodiment 11. The method of any one of embodiments 1 or 3-10, wherein said expanding said population of NK cells is affected under conditions allowing for cell proliferation, wherein said conditions comprise an effective amount of nutrients, serum, growth factors and nicotinamide.

Embodiment 12. The method of embodiment 11, wherein said growth factors comprise at least one growth factor selected from the group consisting of IL-15, IL-2, IL-7, IL-12, IL-21, SCF and FLT3.

Embodiment 13. The method of any one of embodiments 2-12, wherein said effective amount of said nicotinamide comprises an amount between 1.0 mM to 10 mM.

Embodiment 14. The method of any one of embodiments 1-13, wherein said expanding said population of NK cells is affected in the presence of feeder cells or a feeder layer.

Embodiment 15. The method of embodiment 14, wherein said feeder cells comprise irradiated cells.

Embodiment 16. The method of embodiment 14 or 15, wherein said feeder cells comprise T cells or PBMCs.

Embodiment 17. The method of embodiment 16, further comprising a CD3 agonist.

Embodiment 18. The method of any one of embodiments 1-17, wherein said expanding said population of NK cells is affected for 14-16 days.

Embodiment 19. The method of any one of embodiment 7-18, wherein said upregulating expression of said at least one membrane bound protein is affected on day 12-14 from initiation of culture.

Embodiment 20. The method of any one of embodiments 1-19, wherein said upregulating expression of said at least one membrane bound protein is affected by mRNA electroporation.

Embodiment 21. The method of any one of embodiments 1-20, wherein said at least one membrane bound protein is transiently expressed.

Embodiment 22. The method of any one of embodiments 1-21, wherein said at least one membrane bound protein comprises a protein which effects an anti-disease function or survival of said NK cells in vivo.

Embodiment 23. The method of any one of embodiments 1-22, wherein said at least one membrane bound protein is selected from the group consisting of IL-15, IL-15R. Receptor Linker IL-15 (RLI) and TLR.

Embodiment 24. The method of any one of embodiments 1-22, wherein said at least one membrane bound protein comprises a chimeric antigen receptor (CAR) or a transgenic T cell receptor (tg-TCR).

Embodiment 25. The method of embodiment 24, wherein said CAR comprises at least one co-stimulatory domain.

Embodiment 26. The method of embodiment 25, wherein said at least one co-stimulatory domain is selected from the group consisting of CD28, 2B4, CD137/4-1BB, CD134/OX40, Lsk, ICOS and DAP10.

Embodiment 27. The method of any one of embodiments 24-26, wherein said CAR comprises at least one activating domain.

Embodiment 28. The method of embodiment 27, wherein said activating domain comprises a CD35 or FcR-γ.

Embodiment 29. The method of any one of embodiments 24-28, wherein said CAR comprises at least one of a transmembrane domain and a hinge domain.

Embodiment 30. The method of embodiment 29, wherein said transmembrane domain is selected from a CD8, a CD28 and a NKG2D.

Embodiment 31. The method of embodiment 29 or 30, wherein said hinge domain is selected from a CD8 and a CD28.

Embodiment 32. The method of any one of embodiments 24-31, wherein said CAR comprises an antigen binding domain being an antibody or an antigen-binding fragment.

Embodiment 33 The method of embodiment 32, wherein the antigen-binding fragment is a Fab or a scFv.

Embodiment 34. The method of any one of embodiments 24-33, wherein said CAR or said tg-TCR has antigenic specificity for an antigen selected from the group consisting of a tumor antigen, a viral antigen, a bacterial antigen, a fungal antigen, a protozoa antigen, and a parasite antigen.

Embodiment 35. The method of embodiment 34, wherein said tumor antigen is associated with a solid tumor.

Embodiment 36. The method of embodiment 34, wherein said tumor antigen is associated with a hematologic malignancy.

Embodiment 37. The method of any one of embodiments 34-36, wherein said CAR or said tg-TCR has antigenic specificity for an antigen selected from the group consisting of HER2/Neu, CD38, CD19, CD319/CS1, ROR1. CD20, CD5, CD7. CD22, CD70, CD30. BCMA, CD25, NKG2D ligands, MICA/MICB, carcinoembryonic antigen, alphafetoprotein, CA-125, MUC-1, epithelial tumor antigen, melanoma-associated antigen, mutated p53, mutated ras, ERBB2, folate binding protein, HIV-1 envelope glycoprotein gp120, HIV-1 envelope glycoprotein gp41, GD2, CD123, CD23, CD30, CD56, c-Met, mesothelin, GD3. HERV-K, IL-11Ralpha, kappa chain, lambda chain, CSPG4, ERBB2, WT-1, EGFRVIII, TRAIL/DR4, and/or VEGFR2.

Embodiment 38. The method of any one of embodiments 1-37, wherein said at least one membrane bound protein comprises co-expression of:
(i) a CAR or a tg-TCR, and
(ii) a cytokine or a receptor which effects the survival of said NK cells in vivo.

Embodiment 39. The method of any one of embodiments 1 or 3-37, wherein when said gene of interest is CISH, said at least one membrane bound protein comprises IL-15.

Embodiment 40. The method of any one of embodiments 1 or 3-37, wherein when said gene of interest is CD38, said at least one membrane bound protein comprises anti-CD38 CAR.

Embodiment 41. An isolated population of NK cells obtainable according to the method of any one of embodiments 1-40.

Embodiment 42. A pharmaceutical composition comprising the isolated population of NK cells of embodiment 41 and a pharmaceutically active carrier.

Embodiment 43. A method of treating a disease in a subject in need thereof, the method comprising administering to the subject a therapeutically effective amount of the isolated population of NK cells of embodiment 41, thereby treating the subject.

Embodiment 44. A therapeutically effective amount of the isolated population of NK cells of embodiment 41 for use in treating a disease in a subject in need thereof.

Embodiment 45. The method of embodiment 43, or isolated population of NK cells for use of embodiment 44, wherein the disease is selected from the group consisting of a malignant disease, a viral disease, a bacterial disease, a fungal disease, a protozoa disease, and a parasite disease.

Embodiment 46. The method or isolated population of NK cells for use of embodiment 45, wherein said malignant disease is a solid tumor or tumor metastasis.

Embodiment 47. The method or isolated population of NK cells for use of embodiment 46, wherein said malignant disease is selected from the group consisting of a breast cancer, an ovarian cancer, a bladder cancer, a pancreatic cancer, a stomach cancer, a lung cancer, a melanoma, a sarcoma, a neuroblastoma, a colon cancer, a colorectal cancer, an esophageal cancer, a synovial cell cancer, a uterus cancer, a glioma and a cervical cancer.

Embodiment 48. The method or isolated population of NK cells for use of embodiment 45, wherein said malignant disease is a hematological malignancy.

Embodiment 49. The method or isolated population of NK cells for use of embodiment 48, wherein said hematological malignancy comprises a leukemia, a lymphoma or multiple myeloma.

Embodiment 50. The method of any one of embodiments 43 or 45-49, or isolated population of NK cells for use of embodiments 44-49, wherein the subject is a human subject.

### EXAMPLE 1: CISH Gene Knockout (KO) in NAM Ex Vivo Expanded NK Cells

Despite the NK cell inherent ability to kill a broad range of virally infected, stressed and transformed cells, low numbers of dysfunctional NK cells are often observed in many advanced solid human cancers. Accordingly, the present inventors have produced next-generation strategies to enhance NK cell therapy such as by targeting checkpoints that regulate the functionality of NK cells in the tumor microenvironment.

IL-15 is a pleotropic cytokine which is important regulator of NK cell development, homeostasis and activation. IL-15 signaling is required for NK cell proliferation, survival and antitumor function in the tumor microenvironment (as further discussed in Example 2, below).

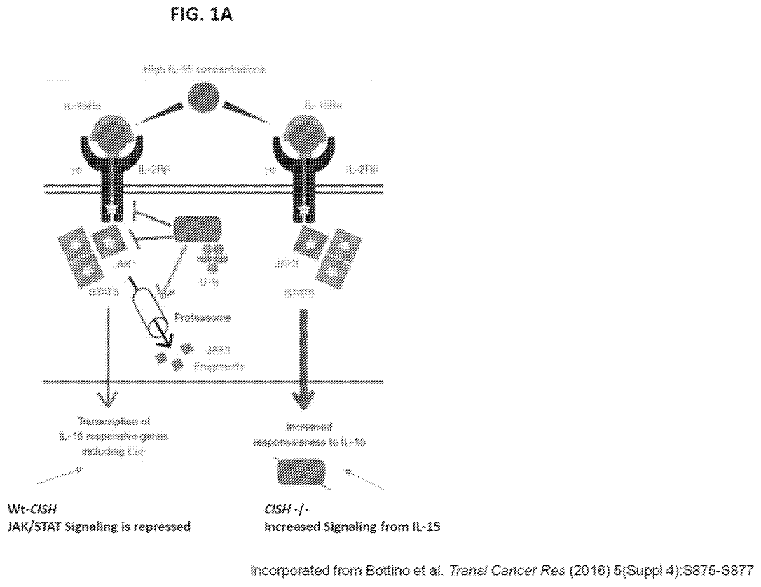

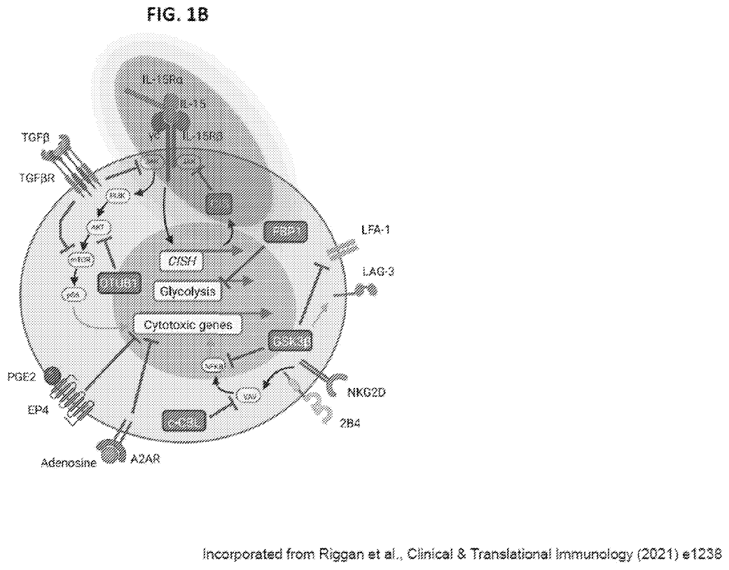

As illustrated in FIGS. 1A-B, the cytokine-inducible SH2-containing protein (CIS, encoded by CISH) is a member of the suppressors of cytokine signaling (SOCS), and acts as a key suppressor of IL-15 signaling via a negative feedback loop. SOCS genes are induced following cytokine receptor engagement and activation of the JAK/STAT signaling cascade. They primarily function as adaptors for an E3 ubiquitin ligase complex and inhibit cytokine signaling by binding to the receptor complex and/or the associated JAK protein tyrosine kinases, targeting them for proteasomal degradation.

Accordingly, the present inventors aimed at generating NK cells in which the CISH gene is knocked out, thereby generating cells which have increased sensitivity IL-15 by lowering the NK activation threshold.

For CISH knockout, four gRNAs targeting 3rd exon and another four gRNAs targeting 4″exon were examined (data not shown). As discussed above, the DNA sequence of each guide was cloned into CRISPR expression plasmid and genome editing experiments were performed in the Hek293 cell line. The most active gRNAs (see Table 1, above) were chosen for further experiments.

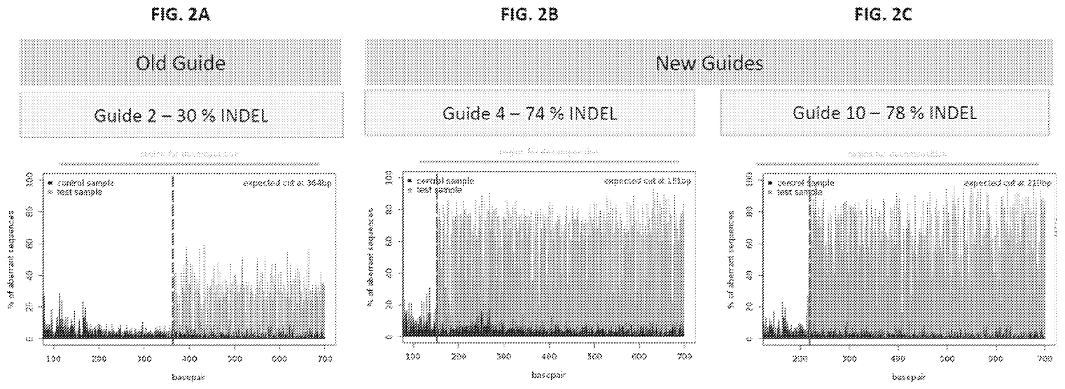

As evident from FIGS. 2A-C, out of three gRNAs tested, Guide 4 (also referred to as CISH-G4 or CISH2) and Guide 10 (also referred to as CISH-G10 or CISH3) yielded high editing rates (85% and 82% INDEL frequency, respectively), while the ‘Old guide’ (CISH1, whose sequence was taken from a public publication, see Palmer et al. bioRxiv Sep. 25, 2020) yielded 30% INDEL frequency. The highest INDEL index was determined for gRNA-4 (CISH2).

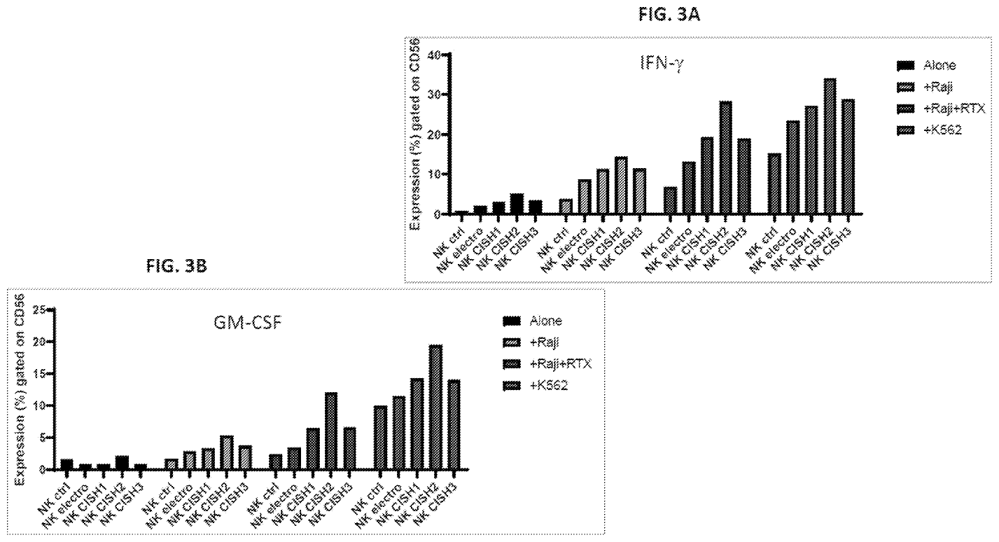

CISH knockout enhanced pro-inflammatory cytokine response as evident in FIGS. 3A-B. Specifically, Guide 4 (CISH2) was found to be the most active and resulted in increased potency, as compared to Guide 10 (CISH3) and to the ‘Old guide’ (CISH 1).

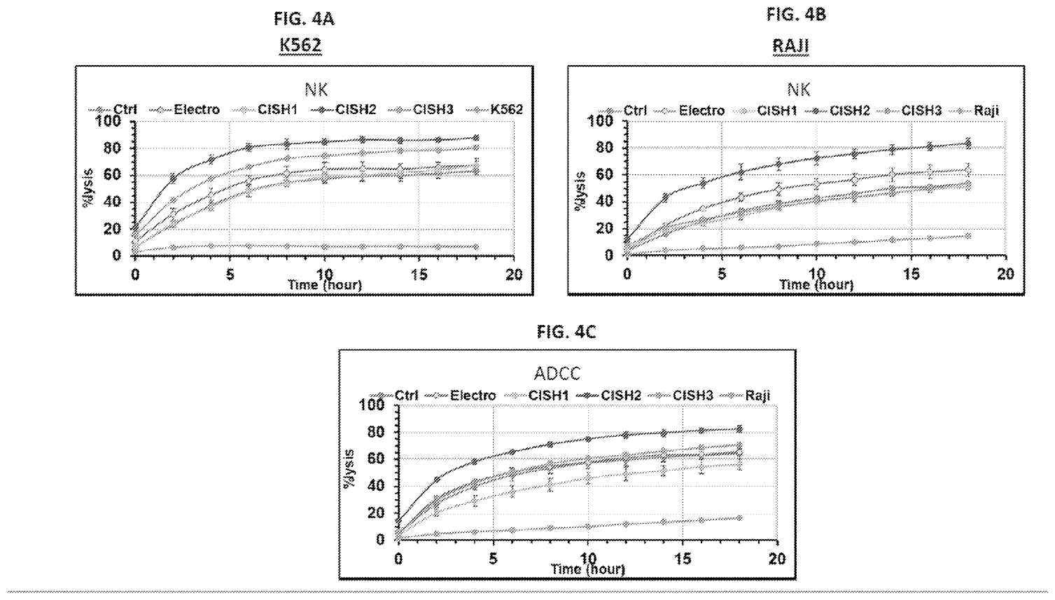

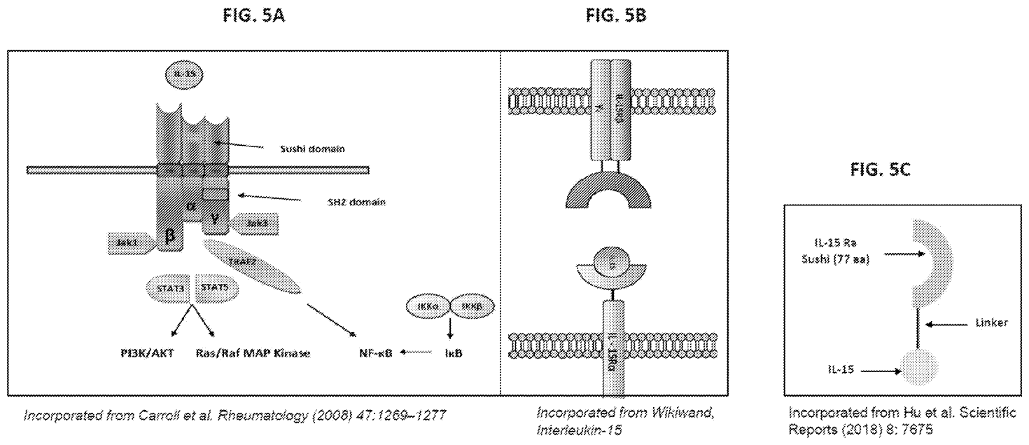

Furthermore, CISH knockout increased cytotoxicity against tumor cell lines as evident in FIGS. 4A-C. Specifically. Guide 4 (CISH2) was found to have the most efficient killing activity against co-cultured target cells.

### EXAMPLE 2: Expression of Membrane-Bound IL-15 on NAM Ex Vivo Expanded NK Cells

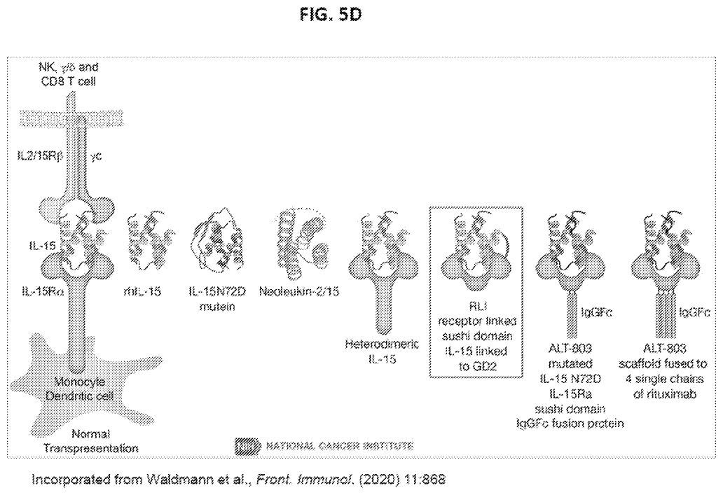

IL-15 is mainly produced by activated myeloid cells as a membrane-bound heterodimer associated with IL-15R˜ in such a way that it is trans-presented to NK cells and T cells expressing IL-2/IL-15RB and the common γ chain receptor (see FIGS. 5A-B). Importantly, IL-15 is critically needed for the ontogeny of NK cells, and in fact is the only cytokine that has been shown to directly support NK cell development in vivo. Thus, IL-15 induces the proliferation, cytotoxic action, and the release of other cytokines such as IFN-γ from NK cells, highlighting its role in potentiating the immune response.

Preclinical observations strongly support the potential antitumor activity of IL-15 mediated by NK cells and T lymphocytes. Systemic delivery of IL-15 has been trialed as a cancer therapy, but enthusiasm for its use has been tempered by toxicity. Thus, new approaches that deliver IL-15 more precisely to the target cell or specifically render the target cell more responsive to IL-15 are appealing avenues for investigation.

### Receptor Linker IL-15 (RLI)

In vitro and in vivo preclinical studies indicate that IL-15 is more bioactive when trans-presented adsorbed onto the IL-15R˜ receptor subunit. Recombinant IL-15 is quickly eliminated from the blood due to its small molecular size (it has an in vivo half-life of 2.5 hours). Several approaches have therefore focused on designing more stable protein constructs encompassing IL-15 and IL-15RE that display a longer half-life and better bio-distribution parameters.

The recombinant protein Receptor Linker IL-15 (RLI) encompasses the binding domain of IL-15R˜ (the so-called sushi domain) bound to IL-15 by a flexible linker (see Figures SC-D). This fusion protein displays longer half-life, super agonistic activity towards the IL-1 SR-β/γ complex, and exerts anti-tumor properties in in-vivo models.

### IL-15 N72D Substitution

Amino acid substitutions of the asparagine residue at position 72, located at the end of helix C, were found to provide both partial agonist and superagonist activity, with various non-conservative substitutions providing enhanced activity. Particularly, the N72D substitution provided a 4-5 fold increased in biological activity of the IL-15 mutein compared to the native molecule based on proliferations assays with cells bearing human IL-15RB and common γ chains (Zhu et al., J Immunol (2009) 183 (6) 3598-3607). The IL-15N72D mutein exhibited superagonist activity through improved binding ability to the human IL-15RB chain. The enhanced biological activity of IL-15N72D was associated with more intense phosphorylation of Jakl and Stat5 and better anti-apoptotic activity compared to the wild-type IL-15. IL-15N72D superagonist activity was also preserved when linked to a single-chain T cell receptor domain to generate a tumor-specific fusion protein (Zhu et al., J Immunol (2009) 183 (6) 3598-3607). Thus, the human IL-15 superagonist muteins and fusions may create opportunities to construct more efficacious immunotherapeutic agents with clinical utility

### Membrane Bound RLI N72D

RLI binds with high affinity to the IL-15β/γ receptors, and amino acid substitution of Asparagine with Aspartic acid at position 72 of the IL-15 cytokine amplified the activity of the receptor ligand contact (Guo et al. Cytokine Growth Factor Rev. (2017) 3 8:10-21).

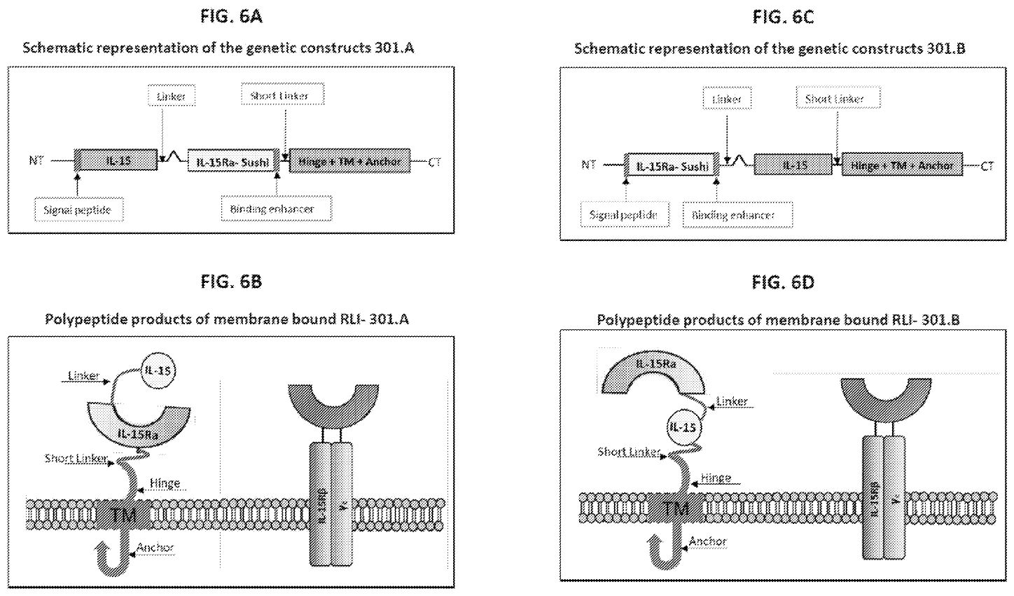

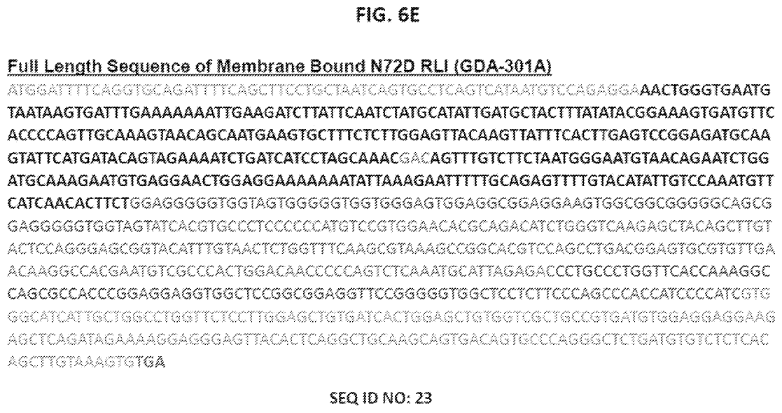

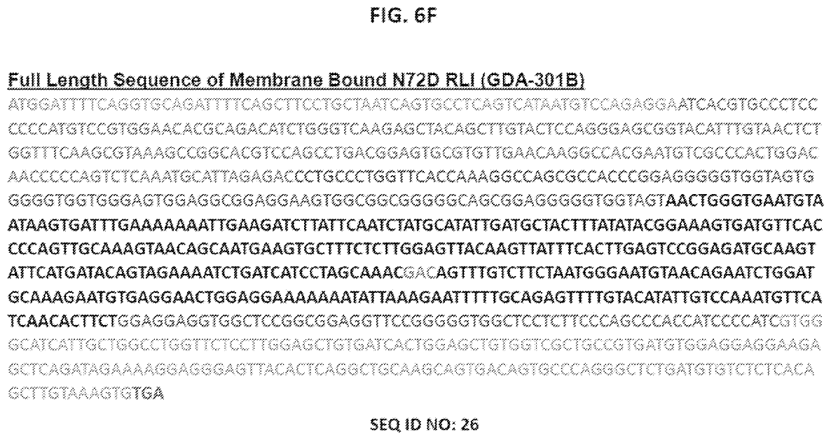

Taking all these features together, the present inventors designed new RLI constructs termed 301.A and 301. B bearing the N72D mutation and linked to the cell membrane with HLA extracellular chain, transmembrane part and cytosolic anchor (see FIGS. 6A-C).

Construction of genetic construct 301.A (FIGS. 6A-B and 6E)

RLI-amino acid substitution of Asparagine with Aspartic acid at position 72 as described by Wong's group (Zhu et al J Immunol (2009) 183 (6) 3598-3607), and set forth in SEQ ID NO: 11.

The linker between IL-15 and IL-15Ra was replaced with a conventional linker of 25 aa-(Gly4 Ser) 5 (SEQ ID NOs: 17-18).

The linker between IL-15Ra and the HLA chain was replaced with conventional short (13 aa) GS linker (SEQ ID NOs: 19-20).

Extracellular, transmembrane, and cytosolic segments were replaced with the human HLA-A gene (SEQ ID NOs: 21-22).

The full construct of 301.A is provided in SEQ ID Nos: 23-24, full sequence and codon optimized sequence, respectively.

Construction of genetic construct 301.B(FIGS. 6C-D and 6F)

RLI-amino acid substitution of Asparagine with Aspartic acid at position 72 as described by Wong's group (Zhu et al., J Immunol (2009) 183 (6) 3598-3607), and set forth in SEQ ID NO: 11.

The linker between IL-15 and IL-15Ra was replace with conventional linker of 25 aa-(Gly4 Ser) 5 (SEQ ID NOs: 17-18).

The linker between IL-15 and the HLA chain was replaced with conventional short (13 aa) GS linker (SEQ ID NOs: 19-20).

Extracellular, transmembrane, and cytosolic segments were replaced with the human HLA-A gene (SEQ ID NOs: 21-22).

The full construct of 301. B is provided in SEQ ID Nos: 26-27, full sequence and codon optimized sequence, respectively.

### Results

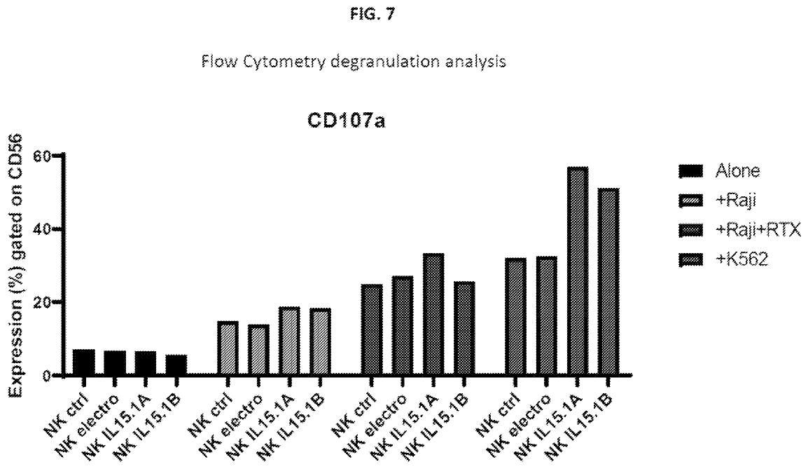

As illustrated in FIG. 7. NK cells expressing membrane bound IL-15 were found to express increased CD 107a related degranulation marker, suggesting high activation of the cells following the manipulation.

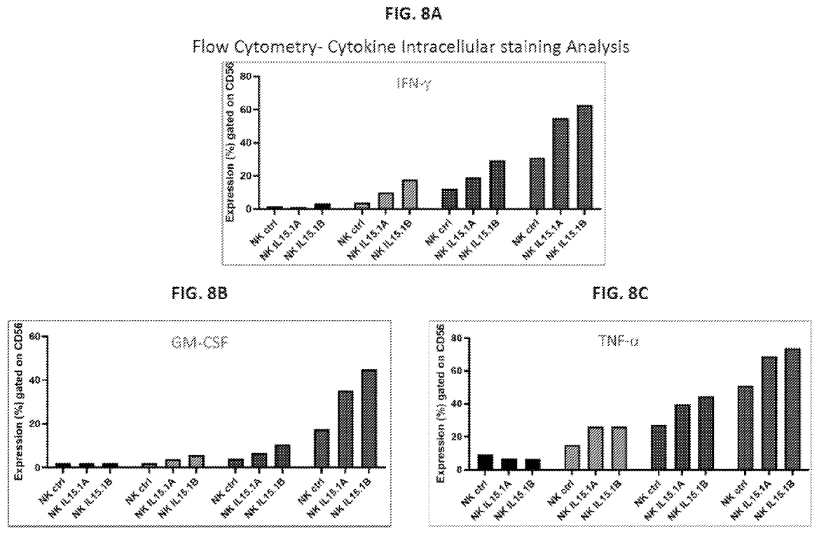

Expression of membrane bound IL-15 also enhanced pro-inflammatory cytokine response following a co-culture with target tumor cell lines as evident in FIGS. 8A-C.

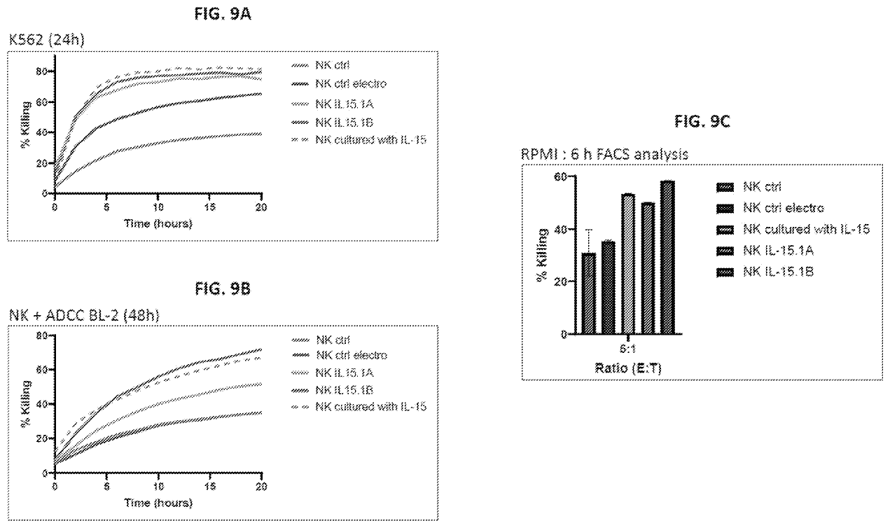

Furthermore, expression of membrane bound IL-15 increased cytotoxicity against the tumor cell lines K562, BL2 and RPMI-8226 as evident in FIGS. 9A-C.

Taken together, RLI construct 301.B illustrated better results between the two constructs evaluated.

### EXAMPLE 3-NAM Ex Vivo Expanded NK Cells Comprising CD38 Gene Knockout (KO) and Expressing Anti-p38 CAR

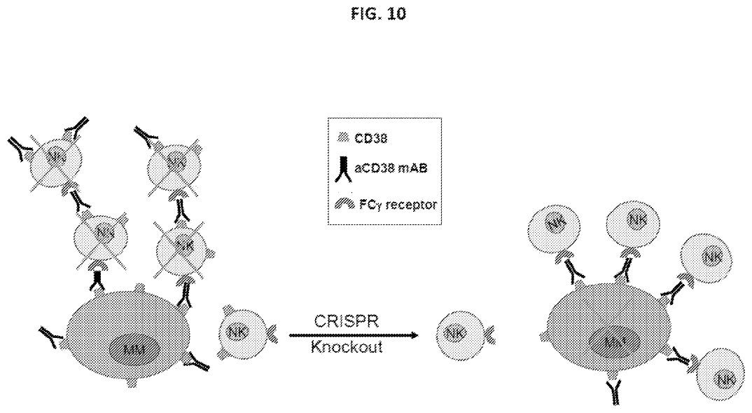

There is a strong biological rationale for the augmentation of allogeneic natural killer (NK) cell therapies with a chimeric antigen receptor (CAR) to enhance multiple myeloma (MM) targeting. CD38 is an established immunotherapeutic target in MM but its expression on NK cells and its further induction during ex vivo NK cell expansion represents a barrier to the development of an anti-CD38 CAR-NK cell therapy (see FIG. 10). To overcome anticipated fratricide due to NK cell CD38 expression when using primary expanded NK cells, the present inventors applied CRISPR-Cas9 genome editing to disrupt CD38 protein expression on NK cells.

For p38 knockout, four gRNAs targeting 2ªd exon were examined (data not shown). As discussed above, the DNA sequence of each guide was cloned into CRISPR expression plasmid and genome editing experiments were performed in the Hek293 cell line. The most active gRNA sequence (see Table 1, above) was chosen for further experiments.

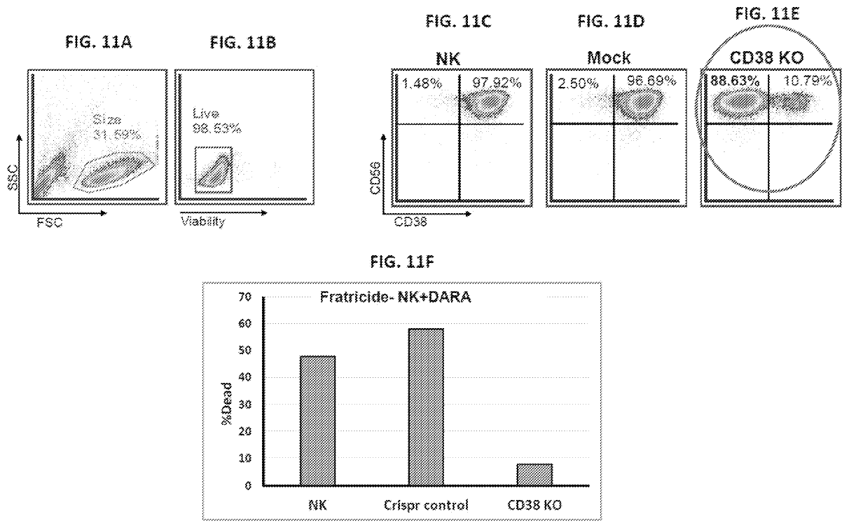

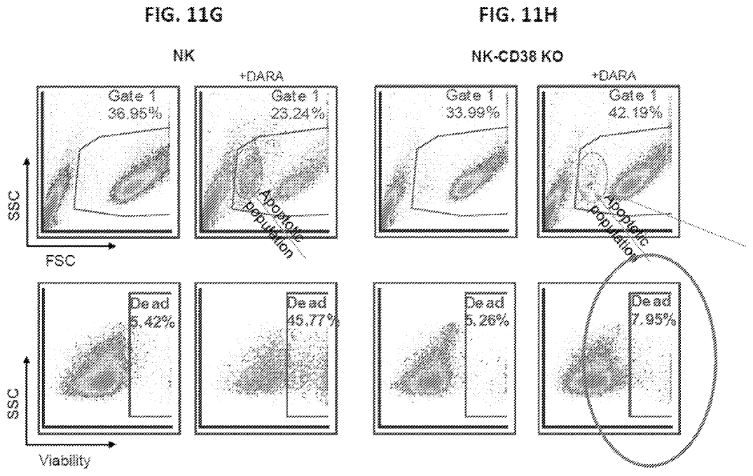

As evident from Figures I LA-E, CD38 KO using CRISPR-Cas9 was efficient and did not affect NK cell viability. Furthermore, CD38 KO NK cells were resistant to fratricide in the presence of Daratumumab (FIG. 11F). However, fratricide rescue was not reflected in better cancer cell killing (FIG. 11F).

To improve cancer therapy an obviate the need for concomitant use of an anti-CD3 8 antibody, such as Daratumumab (DARA), the present inventors further constructed and expressed anti-CD38 CAR on the p38-KO NK cells (see FIG. 13).

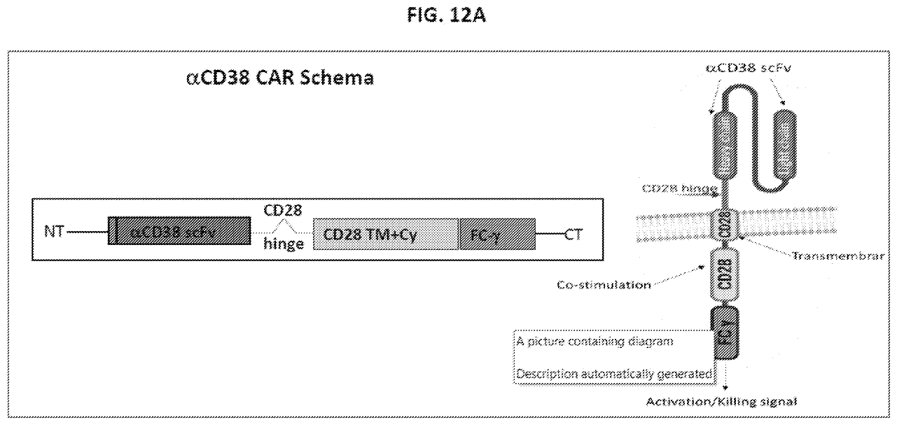

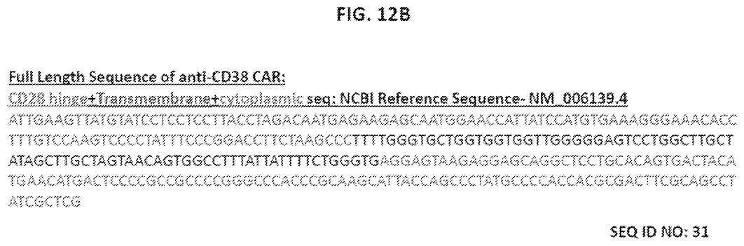

Construction of anti-CD38 CAR (FIGS. 12A-B)

Anti-CD38 CAR was constructed based on single-chain variable fragment (scFv) previously discussed in Zelig et al. (PCT/IL2018/051325 SEQ ID Nos: 29-30). Specifically, the CAR construct was as follows: ˜ CD38scFv-CD28 hinge+TM+Cy-FCy (SEQ ID NOs: 31-32)

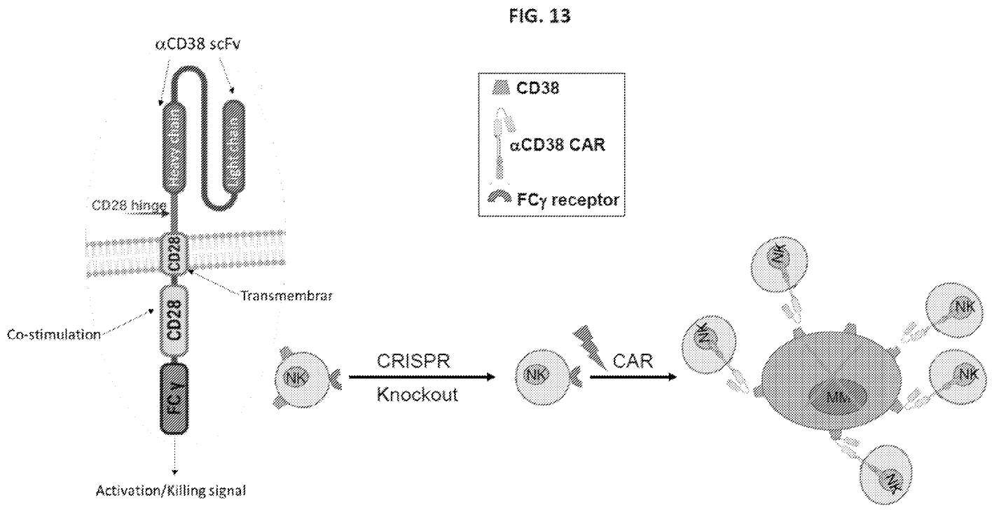

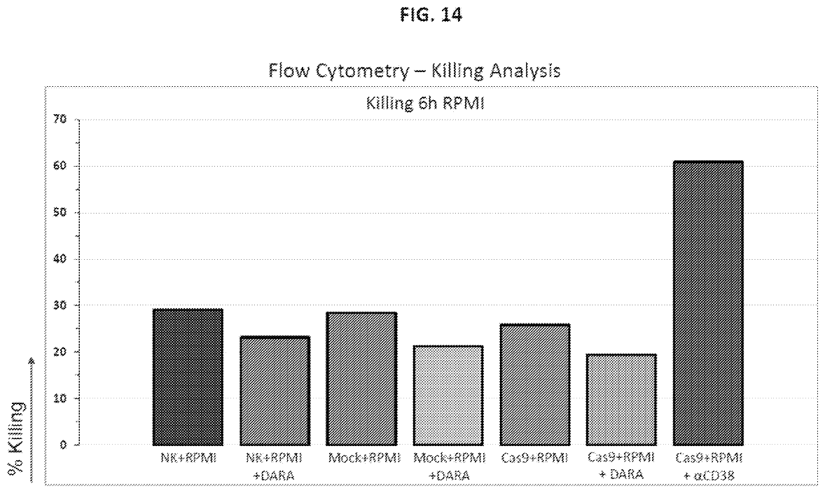

As evident from FIG. 14, the combination of CD38 KO and anti-CD38 CAR expression increased cytotoxicity against multiple myeloma cells.

### EXAMPLE 4: NAM Ex Vivo Expanded Cells Expressing Anti-Her2 CAR

In an attempt to treat solid tumor cancer patients with ERBB2 overexpressing tumors. NK chimeric antigen receptor (CAR) cells were developed based on single-chain variable fragment (scFv) of the widely used humanized monoclonal antibody (mAb) Trastuzumab (Herceptin), as previously discussed by Rosenberg et al. (Mol Ther. (2010) 18 (4): 843+851)

Different fragments of the signaling moieties that attached the Her2 scFv were used. These were expressed on NK cell membranes using mRNA electroporation.

### Anti-HER2 CAR Construction

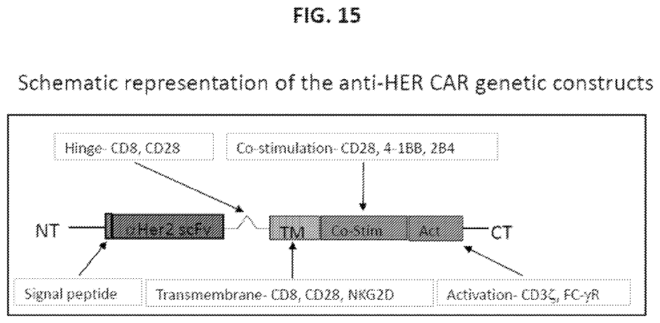

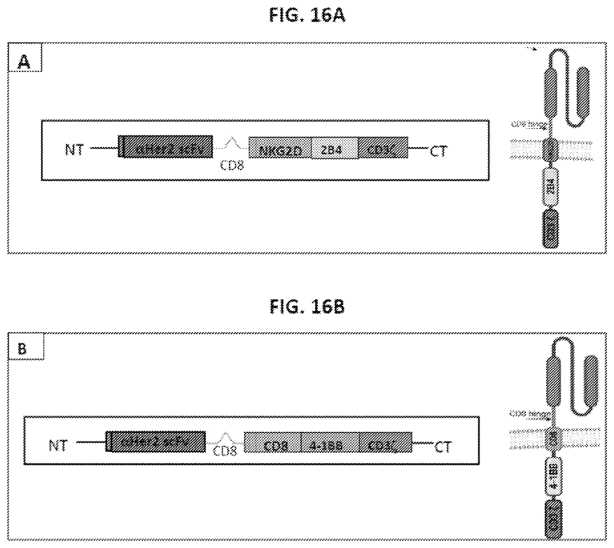

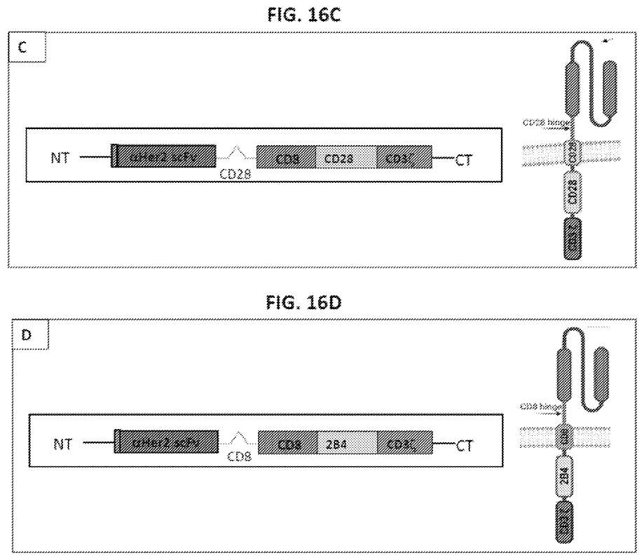

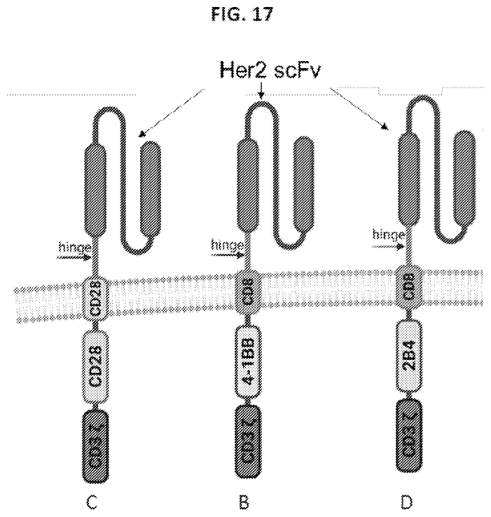

Different constructs were designed for anti-HER2 CAR in a modular way in which the hinge, transmembrane, cytoplasmic domains were modified but the anti-Her2 scFv remained untouched, as depicted in FIGS. 15, 16A-D and 17, and as follows:

### Hinge

The length of the hinge region is important for the formation of the immune synapse. Depending on the antigen distance from the cell surface, the hinge length needs to be adjusted to allow for an optimal distance between the effector and target cell. Amino acid sequences from CD28 or CD8 were used in construction of the anti-HER2 CAR (as specified in SEQ ID Nos: 38-41).

### Transmembrane

The transmembrane (TM) domain consists of a hydrophobic alpha helix that spans the cell membrane and anchors the CAR construct. The choice of TM domain has been shown to affect the functionality of the CAR construct mediated through the degree of cell activation. Amino acid sequences from CD28 or CD8 are most commonly used to date and were used in construction of the anti-HER2 CAR along with the amino acid sequence of NKG2D (as specified in SEQ ID Nos: 42-47).

### Endo-Domains

The evolution of the CAR construct has primarily focused on optimizing the intracellular signaling domains, with the first three generations of CAR constructs referring to the number of activating and co-stimulatory molecules making up the endo-domain. The choice of co-stimulatory domains allows for fine-tuning of the desired NK cell response, whereby CD28-based CARs exhibit an increased cytolytic capacity and shorter persistence compared to 4-1BB-based CARs.

The construction of the anti-HER2 CAR included co-stimulatory domains CD28, 4-1 BB and 2B4 with CD3 3 or FC-receptor activating domain (as specified in SEQ ID Nos: 48-55). The full constructs of anti-HER2 CAR designated A-D are provided in SEQ ID Nos: 60, 62, 64 and 66, respectively.

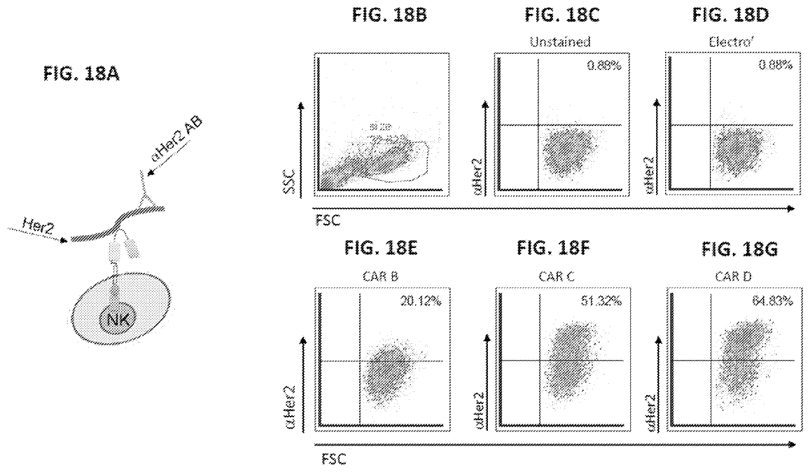

The three CAR constructs designated C, B and D all expressed the anti-HER CAR as evident by the recognition of the HER2 protein (FIGS. 18B-G). Using a sandwich flow cytometry technique, the CAR construct expression was identified on NK cells by pre-incubation of NKs with Erbb2 protein followed by anti-Her2 staining.

### EXAMPLE 5: TGFβ Receptor Gene Knockout (KO) in NAM Ex Vivo Expanded NK Cells

TGFβ is a cytokine that suppresses immune response via the TGFβ receptor. Many tumors overexpress TGFP as an immune defense mechanism.

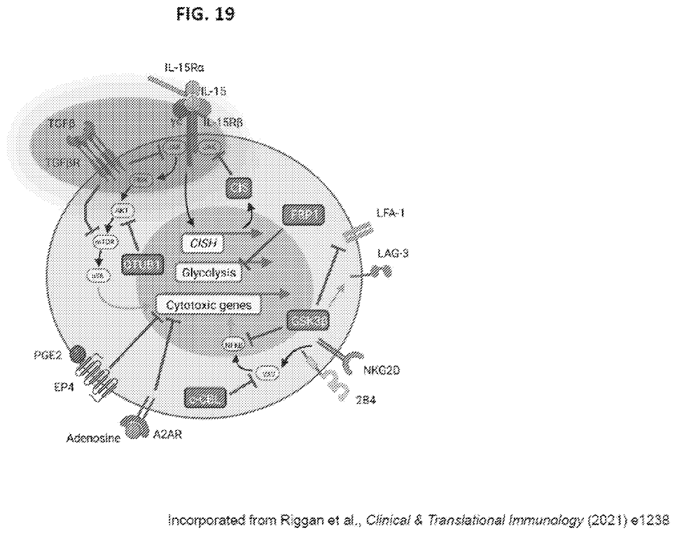

TGFβ receptor 2 knockout renders NK cells insensitive to TGFβ-mediated immunosuppression. Accordingly, the present inventors are generating NK cells in which the TGFβ receptor 2 gene is knocked out (illustrated in FIG. 19).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### EXAMPLE 6: Cell Surface Phenotype in Receptor Linker IL-15 CISH Knockout NK Cells

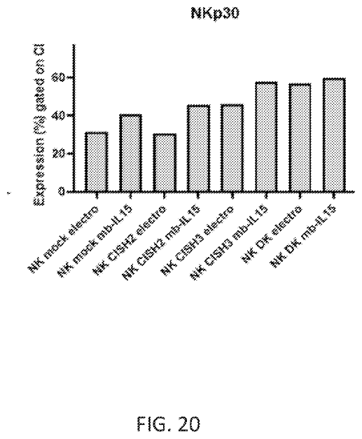

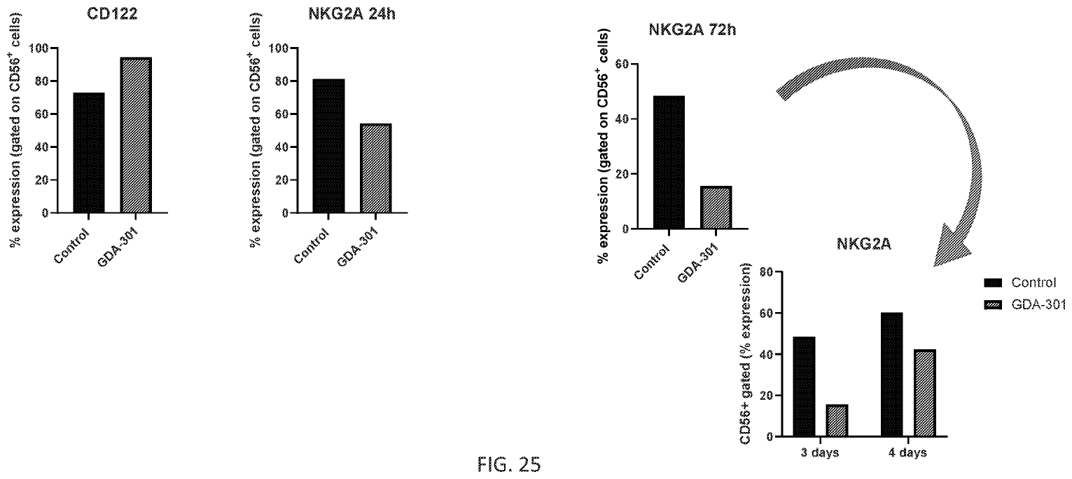

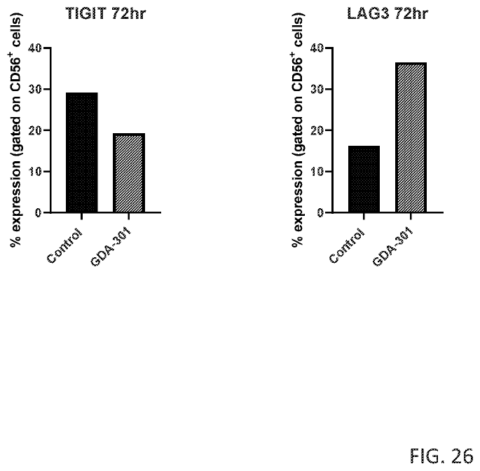

A combined strategy of CISH knockout (KO) and expression of Receptor Linker IL-15 (mbIL-15) results in elevated anti-tumor cytotoxicity. Flow analysis of NKp30 surface expression on CISH KO with co-expression of mbIL-15 is shown in FIG. 20 (guide4=CISH3 and guide3=CISH4). Flow analysis of CD122 and NKG2A surface expression on CISH KO with co-expression of mbIL-15 (GDA-301) is shown in FIG. 25. Moreover, CISH KO with co-expression of mbIL-15 (GDA-301) NK cells demonstrate decreased NKG2A 24-hr post-electroporation, which is maintained over a 4-day period. Flow analysis of TIGIT and LAG3 surface expression on CISH KO with co-expression of mbIL-15 (GDA-301) is shown in FIG. 26.

### EXAMPLE 7: Receptor Linker IL-15 NK Demonstrate Cytotoxicity Against Tumor Cells

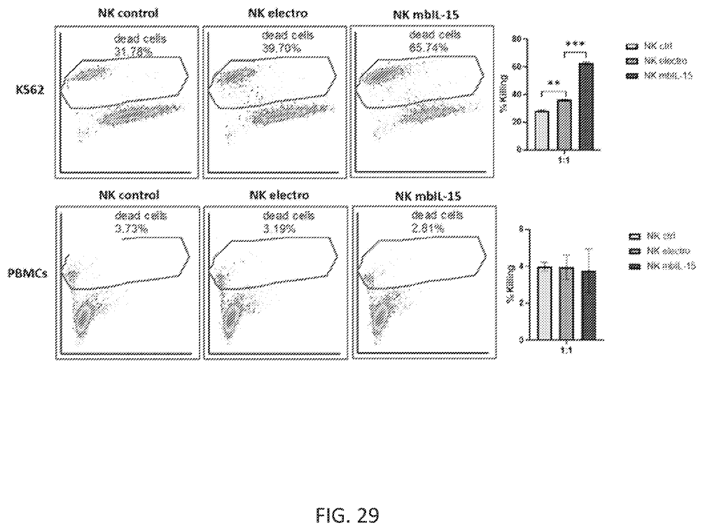

A comparison of the Receptor Linker IL-15 NK (mbIL-15) against a tumor cell line versus allogeneic peripheral blood mononuclear cells was tested. As shown in FIG. 29, significant killing activity was found following a co-culturing of mbIL-15 electroporated NKs with K562 cells (ratio 1:1), as compared to controls.

### EXAMPLE 8: Combined CISH KO and Receptor Linker IL-15 NKs Demonstrate Cytotoxic Potency

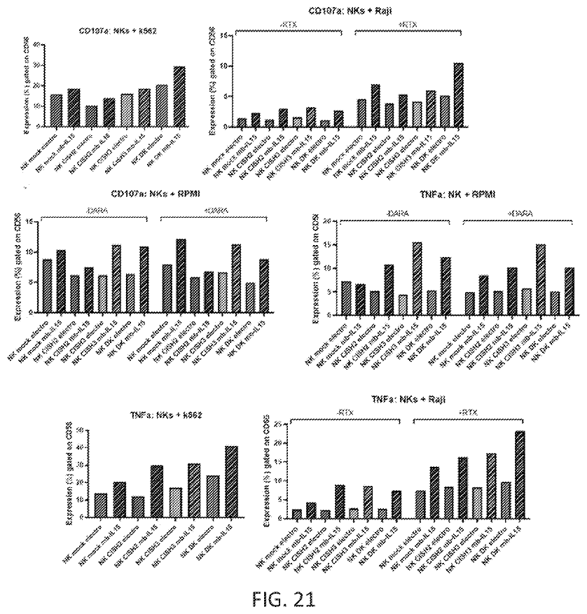

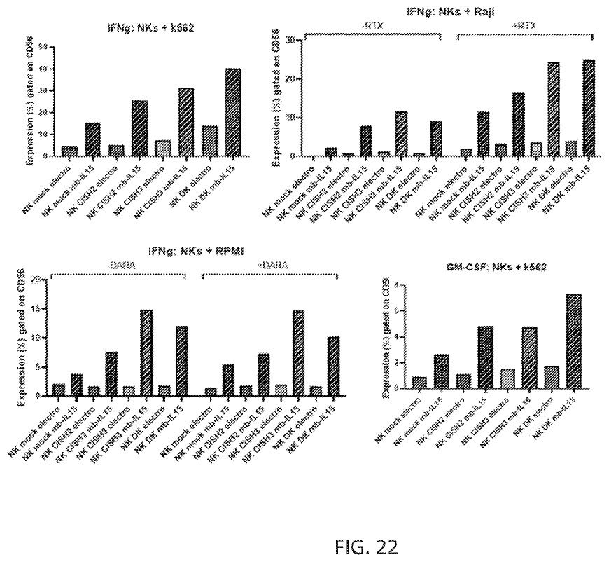

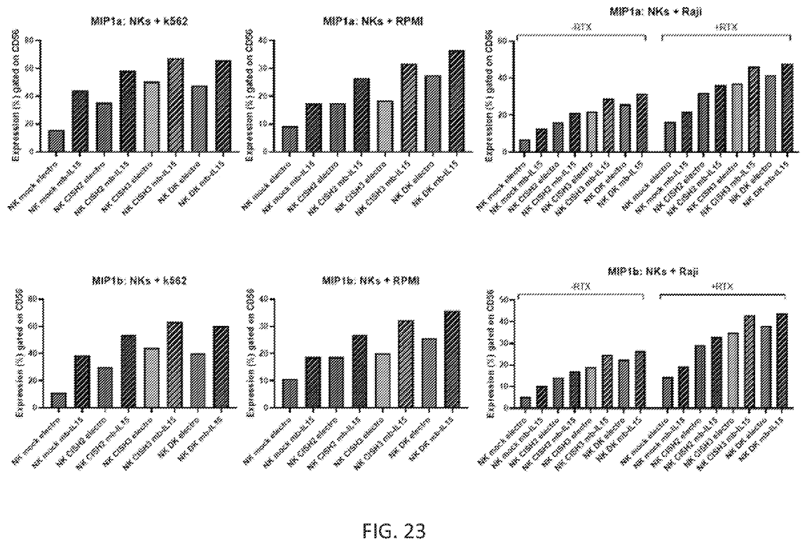

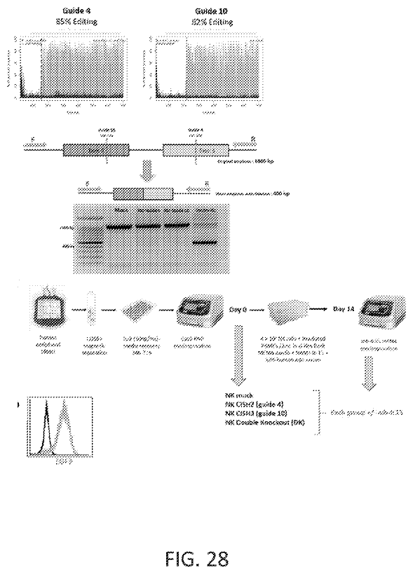

As shown in FIG. 28, CRISPR mediated KO of CISH was confirmed. Additionally, double guide KO (DK) was achieved using both guide4/CISH3 and guide10/CISH4 simultaneously (FIG. 28 gel). The workflow for the generation of NK with CISH KO and mbIL-15 is shown in FIG. 28. An expression of EGFP mRNA served as a positive control for the electroporation process performed at day 14. As shown in FIG. 21 and FIG. 22, CISH KO and mbIL-15 NKs demonstrate cytotoxic potency, following 6-hour co-culture (3:1 E: T) with K562, Raji, and RPMI cells, respectively. Additionally, as shown in FIG. 23, there was also an increase in the expression of MIPIalpha and MIPIbeta in NK cells that express mbIL-15 with CISH KO.

### EXAMPLE 9: Combined CISH KO and Receptor Linker IL-15 NKs Demonstrate Elevated Killing

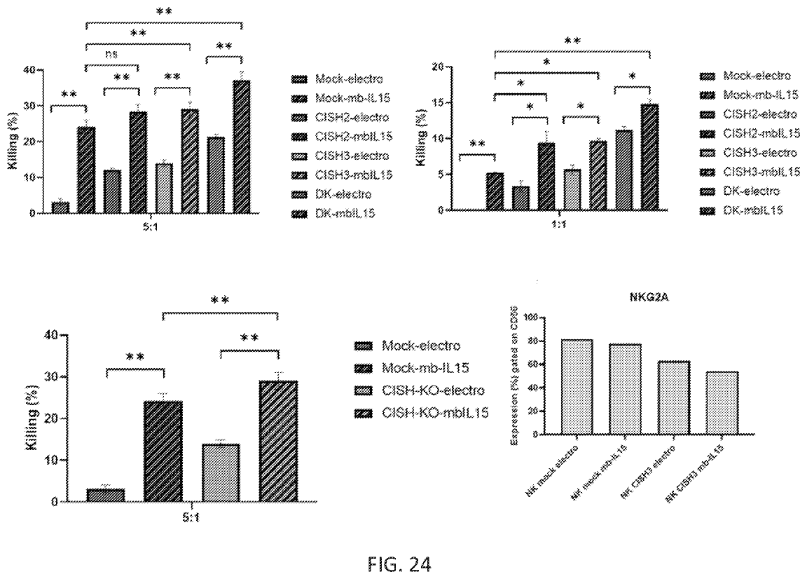

As shown in FIG. 24, elevated killing of K562, Raji, and RPMI cell lines was observed when co-cultured with CISH KO+mbIL-15 NK (1:1 or 5:1 ratio of E.T). K562 and Raji cells were co-cultured with modified NKs for 4-5 hours, whereas RPMI cells were co-cultured with modified NKs for 6 hours.

### EXAMPLE 10: NK Cells with CD38 KO and NK Cells with Combined CD38 KO/CD38 CAR (GDA-601) Expression Demonstrate Low CD38 Expression

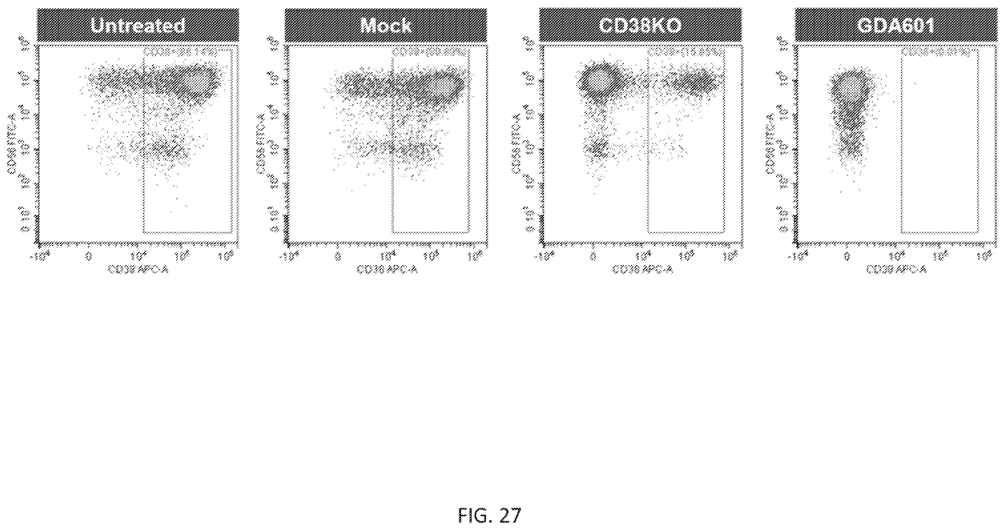

FIG. 27 demonstrates flow analysis of CD38 expression as it relates to NKs with CD38 knocked out (KO) and NKs with combined CD38 KO with CD38 CAR expression.

### EXAMPLE 11: NK Cells with Combined CD38 KO/CD38 CAR (GDA-601) Expression Demonstrate Enhanced Potency and Killing

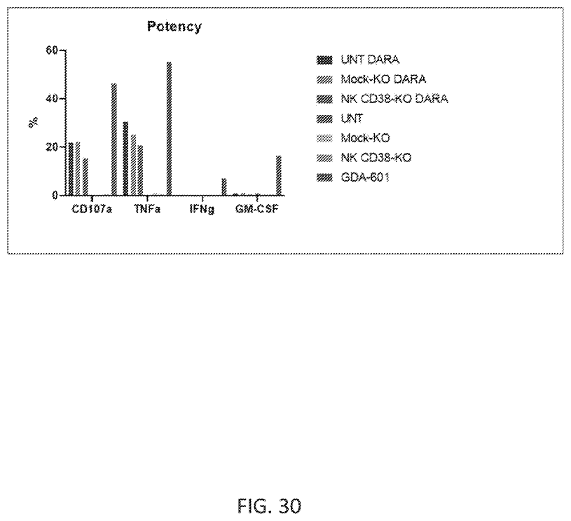

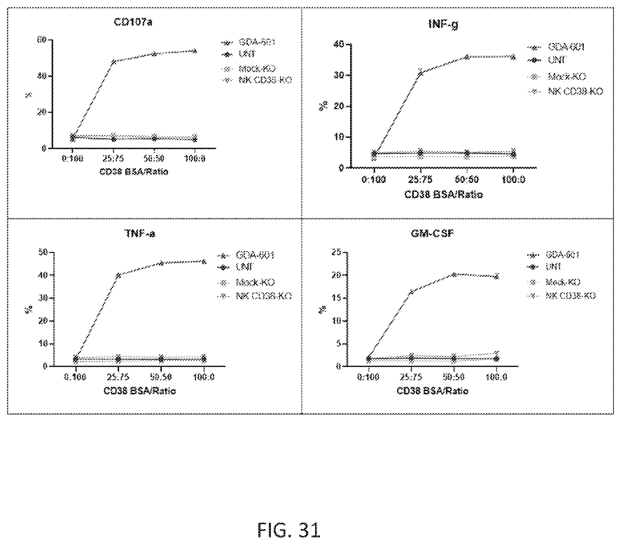

NK cells with combined CD38 KO and CD38 CAR expression were co-cultured with RPMI 8226 cells with and without Daratumumab at an E: T ratio of 1:3 for 6 hours. As shown in FIG. 30, elevated CD107alpha, TNFalpha, IFNgamma, and CM-CSF was observed. FIG. 31 demonstrates the elevated potency of NK cells with combined CD38 KO and CD38 CAR expression when incubated with human recombinant CD38 mixed with BSA pre-coated 6 hours in a plate format.

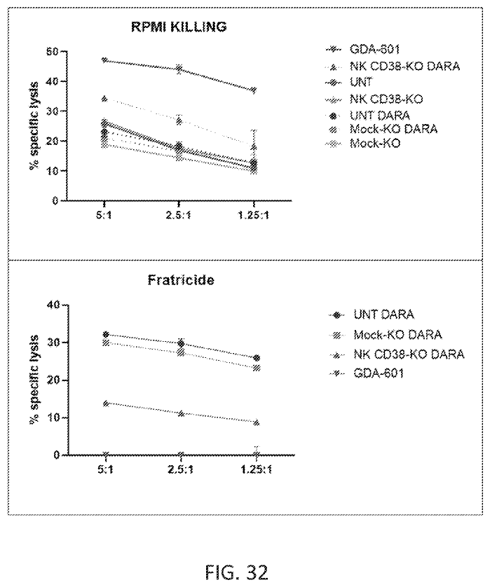

To verify GDA-601 cell killing, GDA-601 cells were cultured with CFSE-labeled RPMI 8226 cells either in the presence or absence of Daratumumab at E: T ratios varying from 5:1 to 0:1 for 6 hours (FIG. 32). Target cells were CFSE gated, and the percentage of dead cells was determined. Alternatively, CFSE negative cells were gated, and the percentage of dead NK cells (fratricide) was determined.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

SEQUENCE LISTING

| SEQ ID | | |
|---|---|---|
| NO | DESCRIPTION | SEQUENCE (5′ to 3′ direction) |
| 1 | CD38-G5 gRNA | TGTAGACTGCCAAAGTGTAT |
| | sequence | |
| 2 | CD38 forward | CCTGGTAGACTGCATGTTAGACGAG |
| | primer | |
| 3 | CD38 reverse | AGCCCCTTTCCTATCTCTGTATCCC |
| | primer | |
| 4 | CISH1 | GGGTTCCATTACGGCCAGCG |
| 5 | CISH-G4 gRNA | GTCCTTTGCTGGCTGTGGAG |
| | sequence (CISH2) | |
| 6 | CISH-G10 gRNA | GTTGGAGTCCAGACGGAAGC |
| | sequence (CISH3) | |
| 7 | CISH forward | CTGCTTCTGCGTACAAAGGGC |
| | primer | |
| 8 | CISH reverse | GGACTAACTGAGCCCATGGCTG |
| | primer | |
| 9 | Human IL-15 NA | ATGAGAATTTCGAAACCACATTTGAGAAGTATTTCCATC |
| | seq-NM_000585.5 | CAGTGCTACTTGTGTTTACTTCTAAACAGTCATTTTCTA |
| | (Mature Protein) | ACTGAAGCTGGCATTCATGTCTTCATTTTGGGCTGTTTC |
| | | AGTGCAGGGCTTCCTAAAACAGAAGCCAACTGGGTGAAT |
| | | GTAATAAGTGATTTGAAAAAAATTGAAGATCTTATTCAA |
| | | TCTATGCATATTGATGCTACTTTATATACGGAAAGTGAT |
| | | GTTCACCCCAGTTGCAAAGTAACAGCAATGAAGTGCTTT |
| | | CTCTTGGAGTTACAAGTTATTTCACTTGAGTCCGGAGAT |
| | | GCAAGTATTCATGATACAGTAGAAAATCTGATCATCCTA |
| | | GCAAACAACAGTTTGTCTTCTAATGGGAATGTAACAGAA |
| | | TCTGGATGCAAAGAATGTGAGGAACTGGAGGAAAAAAAT |
| | | ATTAAAGAATTTTTGCAGAGTTTTGTACATATTGTCCAA |
| | | ATGTTCATCAACACTTCTTGA |
| 10 | Human IL-15 AA | NWVNVISDLKKIEDLIQSMHIDATLYTESDVHPSCKVTA |
| | seq-Mature | MKCFLLELQVISLESGDASIHDTVENLIILANNSLSSNG |
| | Protein | NVTESGCKECEELEEKNIKEFLQSFVHIVQMFINTS |
| 11 | Human IL-15 AA | NWVNVISDLKKIEDLIQSMHIDATLYTESDVHPSCKVTA |
| | seq-Mature | MKCFLLELQVISLESGDASIHDTVENLIILANDSLSSNG |
| | Protein N72D | NVTESGCKECEELEEKNIKEFLQSFVHIVQMFINTS |
| | modification | |
| 12 | Human IL-15Ra - | ATGGCCCCGCGGCGGGCGCGCGGCTGCCGGACCCTCGGT |
| | NM_002189.4 | CTCCCGGCGCTGCTACTGCTGCTGCTGCTCCGGCCGCCG |
| | | GCGACGCGGGGCATCACGTGCCCTCCCCCCATGTCCGTG |
| | | GAACACGCAGACATCTGGGTCAAGAGCTACAGCTTGTAC |
| | | TCCAGGGAGCGGTACATTTGTAACTCTGGTTTCAAGCGT |
| | | AAAGCCGGCACGTCCAGCCTGACGGAGTGCGTGTTGAAC |
| | | AAGGCCACGAATGTCGCCCACTGGACAACCCCCAGTCTC |
| | | AAATGCATTAGAGACCCTGCCCTGGTTCACCAAAGGCCA |
| | | GCGCCACCCTCCACAGTAACGACGGCAGGGGTGACCCCA |
| | | CAGCCAGAGAGCCTCTCCCCTTCTGGAAAAGAGCCCGCA |
| | | GCTTCATCTCCCAGCTCAAACAACACAGCGGCCACAACA |
| | | GCAGCTATTGTCCCGGGCTCCCAGCTGATGCCTTCAAAA |
| | | TCACCTTCCACAGGAACCACAGAGATAAGCAGTCATGAG |
| | | TCCTCCCACGGCACCCCCTCTCAGACAACAGCCAAGAAC |
| | | TGGGAACTCACAGCATCCGCCTCCCACCAGCCGCCAGGT |
| | | GTGTATCCACAGGGCCACAGCGACACCACTGTGGCTATC |
| | | TCCACGTCCACTGTCCTGCTGTGTGGGCTGAGCGCTGTG |
| | | TCTCTCCTGGCATGCTACCTCAAGTCAAGGCAAACTCCC |
| | | CCGCTGGCCAGCGTTGAAATGGAAGCCATGGAGGCTCTG |
| | | CCGGTGACTTGGGGGACCAGCAGCAGAGATGAAGACTTG |
| | | GAAAACTGCTCTCACCACCTATGA |
| 13 | Human IL-15Ra - | ATCACGTGCCCTCCCCCCATGTCCGTGGAACACGCAGAC |
| | Mature Protein | ATCTGGGTCAAGAGCTACAGCTTGTACTCCAGGGAGCGG |
| | NA seq - | TACATTTGTAACTCTGGTTTCAAGCGTAAAGCCGGCACG |
| | Sushi + Exon 3 | TCCAGCCTGACGGAGTGCGTGTTGAACAAGGCCACGAAT |
| | start | GTCGCCCACTGGACAACCCCCAGTCTCAAATGCATTAGA |
| | | GACCCTGCCCTGGTTCACCAAAGGCCAGCGCCACCC |
| 14 | Human IL-15Ra - | ITCPPPMSVEHADIWVKSYSLYSRERYICNSGFKRKAGT |
| | Mature Protein | SSLTECVLNKATNVAHWTTPSLKCIRDPALVHQRPAPP |
| | AA seq - Sushi + | |
| | Exon 3 start | |
| 15 | Leader peptide | ATGGATTTTCAGGTGCAGATTTTCAGCTTCCTGCTAATC |
| | NA seq - anti- | AGTGCCTCAGTCATAATGTCCAGAGGA |
| | body kappa light | |
| | chain | |
| | [Mus musculus]. | |
| | GenBank: | |
| | AB048522.1 | |
| 16 | Leader peptide | MDFQVQIFSFLLISASVIMSRG |
| | AA seq | |
| 17 | Linker- (Gly4 | GGAGGGGGTGGTAGTGGGGGTGGTGGGAGTGGAGGCGGA |
| | Ser)5 - NA seq | GGAAGTGGCGGCGGGGGCAGCGGAGGGGGTGGTAGT |
| 18 | Linker - AA seq | GGGGSGGGGSGGGGSGGGGSGGGGS |
| 19 | Short GS Linker - | GGAGGAGGTGGCTCCGGCGGAGGTTCCGGGGGTGGCTCC |
| | NA seq | |
| 20 | Short GS Linker - | GGGGSGGGSGGGS |
| | AA seq | |
| 21 | HLA - Extra- | TCTTCCCAGCCCACCATCCCCATCGTGGGCATCATTGCT |
| | cellular + | GGCCTGGTTCTCCTTGGAGCTGTGATCACTGGAGCTGTG |
| | Transmembrane + | GTCGCTGCCGTGATGTGGAGGAGGAAGAGCTCAGATAGA |
| | Cytoplasmic | AAAGGAGGGAGTTACACTCAGGCTGCAAGCAGTGACAGT |
| | | GCCCAGGGCTCTGATGTGTCTCTCACAGCTTGTAAAGTG |
| | | TGA |
| 22 | HLA - Extra- | SSQPTIPIVGIIAGLVLLGAVITGAVVAAVMWRRKSSDR |
| | cellular + | KGGSYTQAASSDSAQGSDVSLTACKV |
| | Transmembrane + | |
| | Cytoplasmic-aa- | |
| | NCBI Reference | |
| | Sequence: | |
| | NM_001242758.1 | |
| 23 | Full length | ATGGATTTTCAGGTGCAGATTTTCAGCTTCCTGCTAATC |
| | Membrane Bound | AGTGCCTCAGTCATAATGTCCAGAGGAAACTGGGTGAAT |
| | N72D RLI GDA- | GTAATAAGTGATTTGAAAAAAATTGAAGATCTTATTCAA |
| | 301A - NA seq | TCTATGCATATTGATGCTACTTTATATACGGAAAGTGAT |
| | | GTTCACCCCAGTTGCAAAGTAACAGCAATGAAGTGCTTT |
| | | CTCTTGGAGTTACAAGTTATTTCACTTGAGTCCGGAGAT |
| | | GCAAGTATTCATGATACAGTAGAAAATCTGATCATCCTA |
| | | GCAAACGACAGTTTGTCTTCTAATGGGAATGTAACAGAA |
| | | TCTGGATGCAAAGAATGTGAGGAACTGGAGGAAAAAAAT |
| | | ATTAAAGAATTTTTGCAGAGTTTTGTACATATTGTCCAA |
| | | ATGTTCATCAACACTTCTGGAGGGGGTGGTAGTGGGGGT |
| | | GGTGGGAGTGGAGGCGGAGGAAGTGGCGGCGGGGGCAGC |
| | | GGAGGGGGTGGTAGTATCACGTGCCCTCCCCCCATGTCC |
| | | GTGGAACACGCAGACATCTGGGTCAAGAGCTACAGCTTG |
| | | TACTCCAGGGAGCGGTACATTTGTAACTCTGGTTTCAAG |
| | | CGTAAAGCCGGCACGTCCAGCCTGACGGAGTGCGTGTTG |
| | | AACAAGGCCACGAATGTCGCCCACTGGACAACCCCCAGT |
| | | CTCAAATGCATTAGAGACCCTGCCCTGGTTCACCAAAGG |
| | | CCAGCGCCACCCGGAGGAGGTGGCTCCGGCGGAGGTTCC |
| | | GGGGGTGGCTCCTCTTCCCAGCCCACCATCCCCATCGTG |
| | | GGCATCATTGCTGGCCTGGTTCTCCTTGGAGCTGTGATC |
| | | ACTGGAGCTGTGGTCGCTGCCGTGATGTGGAGGAGGAAG |
| | | AGCTCAGATAGAAAAGGAGGGAGTTACACTCAGGCTGCA |
| | | AGCAGTGACAGTGCCCAGGGCTCTGATGTGTCTCTCACA |
| | | GCTTGTAAAGTGTGA |
| 24 | Codon | ATG GAT TTC CAG GTG CAA ATT TTC AGT TTC |
| | Optimization- | CTC CTC ATC TCT GCT TCC GTA ATT ATG TCC |
| | Full length | CGG GGA AAC TGG GTA AAC GTG ATT TCA GAT |
| | Membrane Bound | CTT AAG AAG ATA GAG GAT TTG ATC CAA AGT |
| | N72D RLI | ATG CAT ATT GAC GCG ACA CTC TAT ACT GAA |
| | GDA-301A - NA | TCA GAC GTT CAT CCT AGT TGC AAG GTT ACG |
| | seq | GCT ATG AAA TGC TTT CTG CTT GAG CTG CAA |
| | | GTT ATA AGT CTG GAG TCA GGC GAT GCT TCT |
| | | ATA CAT GAT ACG GTG GAG AAT TTG ATT ATT |
| | | CTC GCA AAC GAT TCT CTT AGC TCC AAT GGT |
| | | AAT GTG ACA GAG AGC GGT TGC AAG GAG TGT |
| | | GAA GAA CTT GAG GAA AAA AAC ATA AAA GAG |
| | | TTT CTC CAA AGT TTC GTG CAC ATC GTT CAA |
| | | ATG TTT ATA AAT ACG TCA GGA GGT GGT GGA |
| | | AGT GGC GGC GGT GGA TCT GGG GGA GGT GGT |
| | | TCA GGC GGA GGG GGA AGC GGA GGA GGG GGT |
| | | TCT ATC ACT TGT CCG CCC CCA ATG TCC GTG |
| | | GAG CAC GCC GAC ATC TGG GTG AAG TCT TAT |
| | | TCT CTT TAC AGT CGA GAA AGG TAC ATC TGT |
| | | AAC AGT GGC TTT AAA CGC AAA GCT GGT ACC |
| | | AGT TCT CTG ACG GAA TGT GTG CTG AAC AAA |
| | | GCG ACC AAT GTA GCT CAC TGG ACG ACG CCT |
| | | AGT CTC AAG TGC ATT AGG GAT CCC GCT TTG |
| | | GTA CAT CAG AGG CCG GCG CCG CCA GGA GGC |
| | | GGA GGA AGC GGA GGT GGA AGC GGA GGA GGG |
| | | TCA TCA TCA CAG CCC ACT ATT CCA ATC GTC |
| | | GGG ATA ATT GCA GGC CTG GTA TTG CTT GGG |
| | | GCA GTC ATT ACT GGT GCA GTG GTT GCA GCT |
| | | GTG ATG TGG CGA AGA AAG AGC AGC GAC CGG |
| | | AAA GGC GGA AGC TAT ACT CAA GCA GCC TCT |
| | | TCT GAC TCC GCG CAA GGC TCC GAT GTC TCT |
| | | CTC ACT GCG TGT AAG GTA TGA |
| 25 | Full length | MDFQVQIFSFLLISASVIMSRGNWVNVISDLKKIEDLIQ |
| | Membrane Bound | SMHIDATLYTESDVHPSCKVTAMKCFLLELQVISLESGD |
| | N72D RLI GDA- | ASIHDTVENLIILANDSLSSNGNVTESGCKECEELEEKN |
| | 301A - AA seq | IKEFLQSFVHIVQMFINTSGGGGSGGGGSGGGGSGGGGS |
| | | GGGGSITCPPPMSVEHADIWVKSYSLYSRERYICNSGFK |
| | | RKAGTSSLTECVLNKATNVAHWTTPSLKCIRDPALVHQR |
| | | PAPPGGGGSGGGSGGGSSSQPTIPIVGIIAGLVLLGAVI |
| | | TGAVVAAVMWRRKSSDRKGGSYTQAASSDSAQGSDVSLT |
| | | ACKV*X |
| 26 | Full length | ATGGATTTTCAGGTGCAGATTTTCAGCTTCCTGCTAATC |
| | Membrane Bound | AGTGCCTCAGTCATAATGTCCAGAGGAATCACGTGCCCT |
| | N72D RLI GDA- | CCCCCCATGTCCGTGGAACACGCAGACATCTGGGTCAAG |
| | 301B - NA seq | AGCTACAGCTTGTACTCCAGGGAGCGGTACATTTGTAAC |
| | | TCTGGTTTCAAGCGTAAAGCCGGCACGTCCAGCCTGACG |
| | | GAGTGCGTGTTGAACAAGGCCACGAATGTCGCCCACTGG |
| | | ACAACCCCCAGTCTCAAATGCATTAGAGACCCTGCCCTG |
| | | GTTCACCAAAGGCCAGCGCCACCCGGAGGGGGTGGTAGT |
| | | GGGGGTGGTGGGAGTGGAGGCGGAGGAAGTGGCGGCGGG |
| | | GGCAGCGGAGGGGGTGGTAGTAACTGGGTGAATGTAATA |
| | | AGTGATTTGAAAAAAATTGAAGATCTTATTCAATCTATG |
| | | CATATTGATGCTACTTTATATACGGAAAGTGATGTTCAC |
| | | CCCAGTTGCAAAGTAACAGCAATGAAGTGCTTTCTCTTG |
| | | GAGTTACAAGTTATTTCACTTGAGTCCGGAGATGCAAGT |
| | | ATTCATGATACAGTAGAAAATCTGATCATCCTAGCAAAC |
| | | GACAGTTTGTCTTCTAATGGGAATGTAACAGAATCTGGA |
| | | TGCAAAGAATGTGAGGAACTGGAGGAAAAAAATATTAAA |
| | | GAATTTTTGCAGAGTTTTGTACATATTGTCCAAATGTTC |
| | | ATCAACACTTCTGGAGGAGGTGGCTCCGGCGGAGGTTCC |
| | | GGGGGTGGCTCCTCTTCCCAGCCCACCATCCCCATCGTG |
| | | GGCATCATTGCTGGCCTGGTTCTCCTTGGAGCTGTGATC |
| | | ACTGGAGCTGTGGTCGCTGCCGTGATGTGGAGGAGGAAG |
| | | AGCTCAGATAGAAAAGGAGGGAGTTACACTCAGGCTGCA |
| | | AGCAGTGACAGTGCCCAGGGCTCTGATGTGTCTCTCACA |
| | | GCTTGTAAAGTGTGA |
| 27 | Codon | ATG GAT TTT CAA GTA CAA ATT TTT AGT TTC |
| | Optimization- | CTG CTG ATC AGC GCC TCA GTC ATA ATG TCC |
| | Full length | AGG GGT ATC ACG TGT CCA CCC CCG ATG TCC |
| | Membrane Bound | GTA GAG CAC GCT GAT ATT TGG GTA AAG TCC |
| | N72D RLI | TAC TCC CTT TAT TCA AGG GAG CGG TAT ATC |
| | GDA-301.B | TGC AAC AGT GGT TTC AAA CGG AAA GCA GGA |
| | (IDT codon | ACG TCT AGC TTG ACA GAA TGT GTT CTC AAT |
| | optimization | AAA GCT ACG AAT GTC GCC CAT TGG ACC ACT |
| | tool) - NA seq | CCA TCA CTG AAG TGC ATA CGG GAT CCG GCC |
| | | CTG GTA CAC CAA CGG CCT GCT CCT CCT GGT |
| | | GGG GGA GGC TCC GGA GGG GGA GGT TCT GGA |
| | | GGG GGA GGT AGC GGT GGC GGA GGG TCT GGG |
| | | GGA GGC GGG TCC AAC TGG GTG AAC GTC ATA |
| | | TCA GAT TTG AAG AAG ATC GAA GAT CTG ATT |
| | | CAG TCT ATG CAT ATT GAT GCC ACC CTT TAC |
| | | ACG GAA AGT GAT GTT CAC CCA AGT TGT AAG |
| | | GTC ACA GCA ATG AAA TGC TTT TTG CTG GAA |
| | | CTC CAA GTT ATA TCC CTT GAG AGC GGT GAT |
| | | GCG AGC ATC CAT GAT ACC GTT GAA AAT TTG |
| | | ATT ATA CTT GCA AAC GAC AGT TTG AGC TCC |
| | | AAC GGA AAT GTC ACC GAG TCC GGT TGT AAA |
| | | GAG TGC GAG GAG CTC GAG GAG AAA AAT ATT |
| | | AAG GAA TTC CTC CAA TCT TTT GTA CAT ATC |
| | | GTA CAA ATG TTC ATA AAT ACC AGT GGT GGG |
| | | GGT GGT TCC GGG GGA GGC TCA GGT GGC GGT |
| | | AGC TCC AGT CAA CCC ACA ATT CCA ATT GTC |
| | | GGA ATC ATC GCA GGC TTG GTC TTG CTG GGT |
| | | GCC GTC ATA ACG GGG GCA GTT GTC GCG GCA |
| | | GTA ATG TGG CGA AGA AAA TCA TCT GAC CGG |
| | | AAG GGG GGA TCT TAC ACT CAA GCA GCA AGT |
| | | TCA GAC TCA GCC CAA GGG TCT GAC GTA TCC |
| | | TTG ACG GCA TGT AAA GTT TGA |
| 28 | Full length | MDFQVQIFSFLLISASVIMSRGITCPPPMSVEHADIWVK |
| | Membrane Bound | SYSLYSRERYICNSGFKRKAGTSSLTECVLNKATNVAHW |
| | N72D RLI GDA- | TTPSLKCIRDPALVHQRPAPPGGGGSGGGGSGGGGSGGG |
| | 301.B- AA seq | GSGGGGSNWVNVISDLKKIEDLIQSMHIDATLYTESDVH |
| | | PSCKVTAMKCFLLELQVISLESGDASIHDTVENLIILAN |
| | | DSLSSNGNVTESGCKECEELEEKNIKEFLQSFVHIVQMF |
| | | INTSGGGGSGGGSGGGSSSQPTIPIVGIIAGLVLLGAVI |
| | | TGAVVAAVMWRRKSSDRKGGSYTQAASSDSAQGSDVSLT |
| | | ACKV*X |
| 29 | antiCD38 scFv - | GAAATTGTGTTGACGCAGTCTCCAGCCACCCTGTCTTTG |
| | NA seq | TCTCCAGGGGAAAGAGCCACCCTCTCCTGCAGGGCCAGT |
| | | CAGAGTGTTAGCAGCTACTTAGCCTGGTACCAACAGAAA |
| | | CCTGGCCAGGCTCCCAGGCTCCTCATCTATGATGCATCC |
| | | AACAGGGCCACTGGCATTCCAGCCAGGTTCAGTGGCAGT |
| | | GGGTCTGGGACAGACTTCACTCTCACCATCAGCAGCCTA |
| | | GAGCCTGAAGATTTTGCAGTTTATTACTGTCAGCAGCGT |
| | | AGCAACTGGCCTCCGACGTTCGGCCAAGGGACCAAGGTG |
| | | GAAATCAAAGGGTCGACTTCCGGTAGCGGCAAATCCTCT |
| | | GAAGGCAAAGGTGAGGTGCAGCTGTTGGAGTCTGGGGGA |
| | | GGCTTGGTACAGCCTGGGGGGTCCCTGAGACTCTCCTGT |
| | | GCAGTCTCTGGATTCACCTTTAACAGCTTTGCCATGAGC |
| | | TGGGTCCGCCAGGCTCCAGGGAAGGGGCTGGAGTGGGTC |
| | | TCAGCTATTAGTGGTAGTGGTGGTGGCACATACTACGCA |
| | | GACTCCGTGAAGGGCCGGTTCACCATCTCCAGAGACAAT |
| | | TCCAAGAACACGCTGTATCTGCAAATGAACAGCCTGAGA |
| | | GCCGAGGACACGGCCGTATATTTCTGTGCGAAAGATAAG |
| | | ATTTTATGGTTTGGGGAGCCGGTCTTCGACTACTGGGGC |
| | | CAGGGAACCCTGGTCACCGTCTCCTCA |
| 30 | antiCD38 scFv - | EIVLTQSPATLSLSPGERATLSCRASQSVSSYLAWYQQK |
| | AA seq | PGQAPRLLIYDASNRATGIPARFSGSGSGTDFTLTISSL |
| | | EPEDFAVYYCQQRSNWPPTFGQGTKVEIKGSTSGSGKSS |
| | | EGKGEVQLLESGGGLVQPGGSLRLSCAVSGFTFNSFAMS |
| | | WVRQAPGKGLEWVSAISGSGGGTYYADSVKGRFTISRDN |
| | | SKNTLYLQMNSLRAEDTAVYFCAKDKILWFGEPVFDYWG |
| | | QGTLVTVSS |
| 31 | CD28 | ATTGAAGTTATGTATCCTCCTCCTTACCTAGACAATGAG |
| | hinge + Transmem | AAGAGCAATGGAACCATTATCCATGTGAAAGGGAAACAC |
| | brane + cyto- | CTTTGTCCAAGTCCCCTATTTCCCGGACCTTCTAAGCCC |
| | plasmic - NA seq: | TTTTGGGTGCTGGTGGTGGTTGGGGGAGTCCTGGCTTGC |
| | NCBI Reference | TATAGCTTGCTAGTAACAGTGGCCTTTATTATTTTCTGG |
| | Sequence- | GTGAGGAGTAAGAGGAGCAGGCTCCTGCACAGTGACTAC |
| | NM_006139.4 | ATGAACATGACTCCCCGCCGCCCCGGGCCCACCCGCAAG |
| | | CATTACCAGCCCTATGCCCCACCACGCGACTTCGCAGCC |
| | | TATCGCTCG |
| 32 | CD28 - AA seq | IEVMYPPPYLDNEKSNGTIIHVKGKHLCPSPLFPGPSKP |
| | | FWVLVVVGGVLACYSLLVTVAFIIFWVRSKRSRLLHSDY |
| | | MNMTPRRPGPTRKHYQPYAPPRDFAAYRS |
| 33 | Human FC-gamma | CAAGTGCGAAAGGCAGCTATAACCAGCTATGAGAAATCA |
| | chain Activation | GATGGTGTTTACACGGGCCTGAGCACCAGGAACCAGGAG |
| | Domain - NA seq: | ACTTACGAGACTCTGAAGCATGAGAAACCACCACAG |
| | NCBI NM_004106.2 | |
| 34 | Human FC-gamma | QVRKAAITSYEKSDGVYTGLSTRNQETYETLKHEKPPQ |
| | chain Activation | |
| | Domain - AA seq | |
| 35 | Full antiCD38 | ATGGATTTTCAGGTGCAGATTTTCAGCTTCCTGCTAATC |
| | CAR - NA seq | AGTGCCTCAGTCATAATGTCCAGAGGAGAAATTGTGTTG |
| | | ACGCAGTCTCCAGCCACCCTGTCTTTGTCTCCAGGGGAA |
| | | AGAGCCACCCTCTCCTGCAGGGCCAGTCAGAGTGTTAGC |
| | | AGCTACTTAGCCTGGTACCAACAGAAACCTGGCCAGGCT |
| | | CCCAGGCTCCTCATCTATGATGCATCCAACAGGGCCACT |
| | | GGCATTCCAGCCAGGTTCAGTGGCAGTGGGTCTGGGACA |
| | | GACTTCACTCTCACCATCAGCAGCCTAGAGCCTGAAGAT |
| | | TTTGCAGTTTATTACTGTCAGCAGCGTAGCAACTGGCCT |
| | | CCGACGTTCGGCCAAGGGACCAAGGTGGAAATCAAAGGG |
| | | TCGACTTCCGGTAGCGGCAAATCCTCTGAAGGCAAAGGT |
| | | GAGGTGCAGCTGTTGGAGTCTGGGGGAGGCTTGGTACAG |
| | | CCTGGGGGGTCCCTGAGACTCTCCTGTGCAGTCTCTGGA |
| | | TTCACCTTTAACAGCTTTGCCATGAGCTGGGTCCGCCAG |
| | | GCTCCAGGGAAGGGGCTGGAGTGGGTCTCAGCTATTAGT |
| | | GGTAGTGGTGGTGGCACATACTACGCAGACTCCGTGAAG |
| | | GGCCGGTTCACCATCTCCAGAGACAATTCCAAGAACACG |
| | | CTGTATCTGCAAATGAACAGCCTGAGAGCCGAGGACACG |
| | | GCCGTATATTTCTGTGCGAAAGATAAGATTTTATGGTTT |
| | | GGGGAGCCGGTCTTCGACTACTGGGGCCAGGGAACCCTG |
| | | GTCACCGTCTCCTCAGTGAAAGGGAAACACCTTTGTCCA |
| | | AGTCCCCTATTTCCCGGACCTTCTAAGCCCTTTTGGGTG |
| | | CTGGTGGTGGTTGGTGGAGTCCTGGCTTGCTATAGCTTG |
| | | CTAGTAACAGTGGCCTTTATTATTTTCTGGGTGAGGAGT |
| | | AAGAGGAGCAGGCTCCTGCACAGTGACTACATGAACATG |
| | | ACTCCCCGCCGCCCCGGGCCCACCCGCAAGCATTACCAG |
| | | CCCTATGCCCCACCACGCGACTTCGCAGCCTATCGCTCC |
| | | CAAGTGCGAAAGGCAGCTATAACCAGCTATGAGAAATCA |
| | | GATGGTGTTTACACGGGCCTGAGCACCAGGAACCAGGAG |
| | | ACTTACGAGACTCTGAAGCATGAGAAACCACCACAGTAG |
| 36 | Full antiCD38 CAR | ATG GAC TTC CAA GTG CAG ATT TTT TCA TTT |
| | seq - IDT Codon | CTG CTG ATA TCT GCT TCA GTA ATC ATG TCT |
| | Optimization | AGG GGA GAG ATC GTA CTT ACG CAA TCC CCG |
| | | GCA ACC TTG TCT CTC TCA CCC GGG GAA AGG |
| | | GCG ACT CTG TCA TGC AGG GCA AGC CAA AGC |
| | | GTG TCT AGT TAC CTT GCG TGG TAT CAA CAG |
| | | AAG CCA GGA CAA GCT CCC CGG CTC CTG ATT |
| | | TAC GAC GCC TCA AAC CGG GCC ACT GGT ATA |
| | | CCC GCG CGA TTC TCA GGC TCC GGG TCA GGG |
| | | ACT GAC TTT ACT CTC ACA ATC TCC TCC CTC |
| | | GAA CCT GAA GAC TTT GCG GTC TAC TAT TGC |
| | | CAA CAG AGA AGC AAC TGG CCG CCT ACC TTC |
| | | GGG CAA GGA ACT AAA GTG GAG ATT AAG GGC |
| | | TCC ACG TCC GGA AGT GGG AAA TCT TCT GAA |
| | | GGT AAG GGC GAG GTA CAG CTG TTG GAG AGC |
| | | GGA GGT GGC CTG GTA CAA CCG GGA GGA TCT |
| | | CTC CGC TTG TCC TGC GCA GTG TCA GGC TTC |
| | | ACC TTC AAT TCC TTT GCT ATG AGC TGG GTT |
| | | AGA CAA GCC CCC GGG AAG GGT CTG GAA TGG |
| | | GTT TCA GCC ATC TCA GGC TCA GGG GGA GGA |
| | | ACC TAC TAT GCG GAC AGC GTC AAA GGG AGG |
| | | TTT ACG ATT TCA AGG GAC AAT TCT AAA AAT |
| | | ACT CTT TAC CTT CAA ATG AAC TCT TTG CGG |
| | | GCG GAG GAT ACC GCA GTA TAC TTC TGC GCA |
| | | AAG GAC AAA ATC CTC TGG TTC GGA GAA CCC |
| | | GTC TTC GAT TAT TGG GGC CAG GGG ACC TTG |
| | | GTT ACT GTG AGC TCA GTT AAG GGA AAG CAT |
| | | CTG TGT CCC AGC CCA TTG TTC CCG GGG CCG |
| | | TCT AAG CCA TTT TGG GTT CTC GTT GTT GTC |
| | | GGG GGG GTA TTG GCG TGC TAT TCA TTG CTT |
| | | GTC ACT GTG GCC TTT ATA ATA TTT TGG GTA |
| | | CGA TCC AAG AGG AGC CGC CTT TTG CAT AGC |
| | | GAC TAT ATG AAT ATG ACT CCA CGG AGA CCC |
| | | GGT CCA ACA CGC AAG CAT TAT CAG CCA TAC |
| | | GCA CCT CCT CGC GAC TTC GCT GCA TAC AGG |
| | | TCT CAG GTT CGG AAG GCG GCG ATT ACC TCC |
| | | TAC GAG AAA AGC GAC GGA GTG TAC ACG GGA |
| | | TTG TCT ACC CGC AAC CAG GAG ACC TAC GAA |
| | | ACC CTG AAG CAT GAA AAA CCG CCA CAG TAG |
| 37 | Full antiCD38 | MDFQVQIFSFLLISASVIMSRGEIVLTQSPATLSLSPGE |
| | CAR - AA seq | RATLSCRASQSVSSYLAWYQQKPGQAPRLLIYDASNRAT |
| | | GIPARFSGSGSGTDFTLTISSLEPEDFAVYYCQQRSNWP |
| | | PTFGQGTKVEIKGSTSGSGKSSEGKGEVQLLESGGGLVQ |
| | | PGGSLRLSCAVSGFTFNSFAMSWVRQAPGKGLEWVSAIS |
| | | GSGGGTYYADSVKGRFTISRDNSKNTLYLQMNSLRAEDT |
| | | AVYFCAKDKILWFGEPVFDYWGQGTLVTVSSVKGKHLCP |
| | | SPLFPGPSKPFWVLVVVGGVLACYSLLVTVAFIIFWVRS |
| | | KRSRLLHSDYMNMTPRRPGPTRKHYQPYAPPRDFAAYRS |
| | | QVRKAAITSYEKSDGVYTGLSTRNQETYETLKHEKPPQ* |
| | | X |
| 38 | CD28 hinge seq: | ATTGAAGTTATGTATCCTCCTCCTTACCTAGACAATGAG |
| | NCBI Reference | AAGAGCAATGGAACCATTATCCATGTGAAAGGGAAACAC |
| | Sequence- | CTTTGTCCAAGTCCCCTATTTCCCGGACCTTCTAAGCCC |
| | NM_006139.4 | |
| 39 | CD28 hinge aa | IEVMYPPPYLDNEKSNGTIIHVKGKHLCPSPLFPGPSKP |
| | seq | |
| 40 | CD8 hinge seq: | ACCACGACGCCAGCGCCGCGACCACCAACACCGGCGCCC |
| | NCBI Reference | ACCATCGCGTCGCAGCCCCTGTCCCTGCGCCCAGAGGCG |
| | Sequence: | TGCCGGCCAGCGGCGGGGGGCGCAGTGCACACGAGGGGG |
| | NM_001768.7 | CTGGACTTCGCCTGTGAT |
| 41 | CD8 hinge aa | TTTPAPRPPTPAPTIASQPLSLRPEACRPAAGGAVHTRG |
| | seq | LDFACD |
| 42 | CD28 | TTTTGGGTGCTGGTGGTGGTTGGGGGAGTCCTGGCTTGC |
| | transmembrane | TATAGCTTGCTAGTAACAGTGGCCTTTATTATTTTCTGG |
| | seq: NCBI | GTG |
| | Reference | |
| | Sequence- | |
| | NM_006139.4 | |
| 43 | CD28 | FWVLVVVGGVLACYSLLVTVAFIIFWV |
| | transmembrane | |
| | aa seq | |
| 44 | CD8 | ATCTACATCTGGGCGCCCTTGGCCGGGACTTGTGGGGTC |
| | transmembrane | CTTCTCCTGTCACTGGTTATCACC |
| | seq: NCBI | |
| | Reference | |
| | Sequence: | |
| | NM_001768.7 | |
| 45 | CD8 | IYIWAPLAGTCGVLLLSLVIT |
| | transmembrane | |
| | aa seq | |
| 46 | NKG2D | CCATTTTTTTTCTGCTGCTTCATCGCTGTAGCCATGGGA |
| | transmembrane | ATCCGTTTCATTATTATGGTAGCA |
| | seq: NCBI | |
| | Reference | |
| | Sequence: | |
| | NM_007360.4 | |
| 47 | NKG2D | PFFFCCFIAVAMGIRFIIMVA |
| | transmembrane | |
| | aa seq | |
| 48 | CD28 endo | AGGAGTAAGAGGAGCAGGCTCCTGCACAGTGACTACATG |
| | domain seq: NCBI | AACATGACTCCCCGCCGCCCCGGGCCCACCCGCAAGCAT |
| | Reference | TACCAGCCCTATGCCCCACCACGCGACTTCGCAGCCTAT |
| | Sequence- | CGCTCG |
| | NM 006139.4 | |
| 49 | CD28 endo | RSKRSRLLHSDYMNMTPRRPGPTRKHYQPYAPPRDFAAY |
| | domain aa seq | RS |
| 50 | 4-1BB endo | AAACGGGGCAGAAAGAAACTCCTGTATATATTCAAACAA |
| | domain seq: NCBI | CCATTTATGAGACCAGTACAAACTACTCAAGAGGAAGAT |
| | Reference | GGCTGTAGCTGCCGATTTCCAGAAGAAGAAGAAGGAGGA |
| | Sequence: | TGTGAACTG |
| | NM_001561.6 | |
| 51 | 4-1BB endo | KRGRKKLLYIFKQPFMRPVQTTQEEDGCSCRFPEEEEGG |
| | domain aa seq | CEL |
| 52 | 2B4 endo domain | TGGAGGAGAAAGAGGAAGGAGAAGCAGTCAGAGACCAGT |
| | seq: NCBI | CCCAAGGAATTTTTGACAATTTACGAAGATGTCAAGGAT |
| | Reference | CTGAAAACCAGGAGAAATCACGAGCAGGAGCAGACTTTT |
| | Sequence: | CCTGGAGGGGGGAGCACCATCTACTCTATGATCCAGTCC |
| | NM_016382.4 | CAGTCTTCTGCTCCCACGTCACAAGAACCTGCATATACA |
| | | TTATATTCATTAATTCAGCCTTCCAGGAAGTCTGGATCC |
| | | AGGAAGAGGAACCACAGCCCTTCCTTCAATAGCACTATC |
| | | TATGAAGTGATTGGAAAGAGTCAACCTAAAGCCCAGAAC |
| | | CCTGCTCGATTGAGCCGCAAAGAGCTGGAGAACTTTGAT |
| | | GTTTATTCC |
| 53 | 2B4 endo domain | WRRKRKEKQSETSPKEFLTIYEDVKDLKTRRNHEQEQTF |
| | aa seq | PGGGSTIYSMIQSQSSAPTSQEPAYTLYSLIQPSRKSGS |
| | | RKRNHSPSFNSTIYEVIGKSQPKAQNPARLSRKELENFD |
| | | VYS |
| 54 | CD3zetta endo | AGAGTGAAGTTCAGCAGGAGCGCAGACGCCCCCGCGTAC |
| | domain seq: NCBI | CAGCAGGGCCAGAACCAGCTCTATAACGAGCTCAATCTA |
| | Reference | GGACGAAGAGAGGAGTACGATGTTTTGGACAAGAGACGT |
| | Sequence: | GGCCGGGACCCTGAGATGGGGGGAAAGCCGCAGAGAAGG |
| | NM_198053.3 | AAGAACCCTCAGGAAGGCCTGTACAATGAACTGCAGAAA |
| | | GATAAGATGGCGGAGGCCTACAGTGAGATTGGGATGAAA |
| | | GGCGAGCGCCGGAGGGGCAAGGGGCACGATGGCCTTTAC |
| | | CAGGGTCTCAGTACAGCCACCAAGGACACCTACGACGCC |
| | | CTTCACATGCAGGCCCTGCCCCCTCGC |
| 55 | CD3zetta endo | RVKFSRSADAPAYQQGQNQLYNELNLGRREEYDVLDKRR |
| | domain aa seq | GRDPEMGGKPQRRKNPQEGLYNELQKDKMAEAYSEIGMK |
| | | GERRRGKGHDGLYQGLSTATKDTYDALHMQALPPR |
| 56 | Fc fragment of | CAAGTGCGAAAGGCAGCTATAACCAGCTATGAGAAATCA |
| | IgE receptor Ig | GATGGTGTTTACACGGGCCTGAGCACCAGGAACCAGGAG |
| | (FCER1G): NCBI | ACTTACGAGACTCTGAAGCATGAGAAACCACCACAG |
| | Reference | |
| | Sequence: | |
| | NM_004106.2 | |
| 57 | Fc fragment of | QVRKAAITSYEKSDGVYTGLSTRNQETYETLKHEKPPQ |
| | IgE receptor Ig | |
| | aa seq | |
| 58 | Leader Peptide | ATGGATTTTCAGGTGCAGATTTTCAGCTTCCTGCTAAT |
| | seq: GenBank: | CAGTGCCTCAGTCATAATGTCCAGAGGA |
| | KF419288.1 | |
| 59 | Leader Peptide | MDFQVQIFSFLLISASVIMSRG |
| | aa seq | |
| 60 | GDA-501.A: anti- | ATG GAT TTT CAG GTA CAA ATT TTC TCA TTC |
| | Her2 scFv- | CTT CTC ATT TCA GCA TCC GTA ATA ATG TCC |
| | CD8hinge-NKG2D | AGA GGC GAC ATC CAA ATG ACT CAA TCA CCA |
| | TM-2B4-CD3zetta | AGT TCC TTG TCC GCG TCC GTT GGG GAT AGA |
| | Full Seq codon | GTA ACC ATA ACC TGT AGG GCA TCA CAA GAT |
| | optimization | GTG AAT ACG GCC GTA GCG TGG TAT CAA CAG |
| | | AAA CCA GGA AAG GCT CCA AAA CTC CTT ATT |
| | | TAT TCC GCG AGT TTT CTT TAC AGC GGA GTC |
| | | CCG AGT AGA TTC TCT GGT TCT CGC TCT GGC |
| | | ACT GAT TTT ACT CTG ACC ATA AGT TCA CTG |
| | | CAA CCT GAA GAC TTT GCG ACC TAT TAC TGT |
| | | CAA CAG CAC TAC ACA ACG CCT CCC ACT TTT |
| | | GGA CAA GGT ACT AAA GTA GAG ATT AAA CGG |
| | | ACC GGA TCC ACA AGT GGG AGC GGG AAG CCT |
| | | GGT TCT GGC GAA GGA TCC GAA GTT CAG CTG |
| | | GTT GAA TCC GGC GGT GGG TTG GTG CAA CCC |
| | | GGG GGG AGC CTG CGC CTC AGC TGT GCC GCG |
| | | AGT GGG TTT AAC ATA AAA GAT ACC TAC ATT |
| | | CAC TGG GTG CGC CAA GCT CCG GGC AAA GGA |
| | | CTT GAA TGG GTC GCG AGG ATC TAC CCG ACC |
| | | AAC GGT TAC ACA AGA TAT GCG GAC TCC GTA |
| | | AAA GGG CGA TTC ACG ATA TCC GCT GAC ACA |
| | | TCC AAG AAC ACG GCG TAC TTG CAA ATG AAT |
| | | TCT CTT AGG GCC GAG GAC ACC GCA GTT TAT |
| | | TAC TGT AGT CGC TGG GGA GGT GAT GGA TTT |
| | | TAT GCG ATG GAC GTT TGG GGG CAA GGG ACG |
| | | CTG GTC ACG GTT TCC AGT GCT GCA ACC ACA |
| | | ACG CCA GCA CCA AGA CCT CCC ACA CCC GCT |
| | | CCT ACC ATC GCT TCA CAA CCC CTT TCT CTG |
| | | CGA CCA GAG GCG TGT AGA CCC GCC GCT GGG |
| | | GGC GCC GTT CAC ACG AGG GGC CTG GAC TTC |
| | | GCG TGC GAC CCC TTT TTC TTC TGC TGC TTT |
| | | ATA GCT GTG GCG ATG GGA ATT CGA TTT ATA |
| | | ATT ATG GTG GCA TGG AGA CGG AAG CGG AAG |
| | | GAG AAA CAG TCC GAG ACT AGC CCG AAG GAG |
| | | TTC TTG ACC ATT TAT GAA GAC GTA AAA GAT |
| | | TTG AAG ACC CGG CGC AAT CAC GAA CAA GAA |
| | | CAA ACG TTT CCA GGA GGC GGT AGT ACT ATA |
| | | TAC TCA ATG ATT CAA AGT CAA TCT TCA GCA |
| | | CCG ACT TCT CAA GAA CCC GCA TAT ACT CTC |
| | | TAT AGC CTG ATT CAA CCC TCA CGG AAG TCA |
| | | GGG AGC AGG AAA AGG AAC CAT TCA CCG AGT |
| | | TTT AAT TCC ACG ATT TAC GAG GTG ATA GGC |
| | | AAG AGC CAG CCT AAG GCC CAG AAC CCG GCA |
| | | AGA TTG TCC CGA AAG GAA CTC GAA AAC TTT |
| | | GAT GTG TAC TCT AGG GTG AAA TTC AGT CGG |
| | | TCA GCA GAT GCC CCT GCA TAT CAA CAA GGA |
| | | CAA AAC CAA CTG TAT AAC GAG CTC AAT CTT |
| | | GGT CGA CGC GAG GAA TAC GAC GTA CTT GAC |
| | | AAG AGA AGA GGA AGA GAC CCC GAG ATG GGG |
| | | GGC AAG CCG CAG CGA AGA AAG AAT CCA CAA |
| | | GAA GGG CTC TAC AAT GAA CTG CAG AAG GAC |
| | | AAA ATG GCC GAA GCC TAT TCC GAG ATC GGA |
| | | ATG AAG GGT GAG CGA AGG CGA GGA AAG GGG |
| | | CAC GAC GGC CTT TAC CAG GGT CTG TCA ACC |
| | | GCT ACG AAA GAC ACG TAC GAC GCC CTG CAC |
| | | ATG CAA GCG CTC CCA CCA CGA TAG |
| 61 | GDA-501.A: anti- | MDFQVQIFSFLLISASVIMSRGDIQMTQSPSSLSASVGD |
| | Her2 scFv- | RVTITCRASQDVNTAVAWYQQKPGKAPKLLIYSASFLYS |
| | CD8hinge-NKG2D | GVPSRFSGSRSGTDFTLTISSLQPEDFATYYCQQHYTTP |
| | TM-2B4-CD3zetta | PTFGQGTKVEIKRTGSTSGSGKPGSGEGSEVQLVESGGG |
| | Full AA Seq | LVQPGGSLRLSCAASGFNIKDTYIHWVRQAPGKGLEWVA |
| | | RIYPTNGYTRYADSVKGRFTISADTSKNTAYLQMNSLRA |
| | | EDTAVYYCSRWGGDGFYAMDVWGQGTLVTVSSAATTTPA |
| | | PRPPTPAPTIASQPLSLRPEACRPAAGGAVHTRGLDFAC |
| | | DPFFFCCFIAVAMGIRFIIMVAWRRKRKEKQSETSPKEF |
| | | LTIYEDVKDLKTRRNHEQEQTFPGGGSTIYSMIQSQSSA |
| | | PTSQEPAYTLYSLIQPSRKSGSRKRNHSPSFNSTIYEVI |
| | | GKSQPKAQNPARLSRKELENFDVYSRVKFSRSADAPAYQ |
| | | QGQNQLYNELNLGRREEYDVLDKRRGRDPEMGGKPQRRK |
| | | NPQEGLYNELQKDKMAEAYSEIGMKGERRRGKGHDGLYQ |
| | | GLSTATKDTYDALHMQALPPR |
| 62 | GDA-501.B: anti- | ATG GAC TTC CAA GTG CAG ATA TTC TCC TTT |
| | Her2 scFv- | CTT CTT ATT TCC GCA TCC GTT ATA ATG AGC |
| | CD8hinge + TM-4- | AGG GGG GAT ATA CAA ATG ACT CAG TCT CCG |
| | 1BB-CD3zetta | TCC TCT CTG AGT GCA TCC GTG GGC GAC CGA |
| | Full Seq codon | GTA ACT ATC ACG TGC CGG GCA TCC CAA GAT |
| | optimization | GTA AAC ACA GCC GTA GCT TGG TAC CAA CAA |
| | | AAA CCA GGT AAA GCT CCT AAA TTG CTT ATT |
| | | TAC TCT GCA AGT TTC TTG TAC AGT GGG GTG |
| | | CCG TCC CGG TTT AGT GGC TCC AGA AGC GGC |
| | | ACT GAT TTC ACT CTG ACA ATA TCT TCT CTT |
| | | CAA CCC GAG GAT TTT GCT ACA TAT TAT TGC |
| | | CAA CAA CAT TAT ACG ACT CCC CCT ACT TTC |
| | | GGC CAA GGG ACT AAA GTC GAG ATC AAA CGA |
| | | ACC GGT TCC ACG TCC GGG TCA GGT AAA CCT |
| | | GGT TCA GGA GAA GGA TCA GAG GTG CAA TTG |
| | | GTC GAG AGT GGC GGG GGA TTG GTA CAA CCA |
| | | GGT GGC AGT CTT AGG CTG TCT TGT GCA GCC |
| | | TCA GGC TTT AAC ATT AAG GAC ACG TAC ATA |
| | | CAC TGG GTC CGA CAA GCA CCG GGC AAG GGT |
| | | CTG GAA TGG GTG GCA CGG ATT TAT CCT ACA |
| | | AAC GGA TAT ACT CGA TAC GCG GAT TCA GTC |
| | | AAG GGG CGC TTT ACC ATA AGT GCC GAC ACT |
| | | AGC AAG AAC ACT GCC TAT CTT CAG ATG AAT |
| | | TCA TTG AGG GCG GAA GAC ACT GCG GTA TAC |
| | | TAT TGC TCT AGA TGG GGA GGA GAC GGC TTT |
| | | TAT GCA ATG GAT GTG TGG GGT CAG GGA ACC |
| | | CTC GTT ACC GTA TCT TCT GCT GCA ACC ACC |
| | | ACC CCC GCC CCC CGA CCA CCG ACA CCA GCA |
| | | CCA ACA ATC GCA TCC CAG CCT TTG TCA TTG |
| | | AGA CCA GAG GCG TGT AGA CCC GCA GCT GGA |
| | | GGC GCA GTC CAT ACG CGG GGG CTG GAT TTT |
| | | GCC TGC GAC ATA TAT ATA TGG GCT CCT CTC |
| | | GCG GGT ACG TGC GGT GTT TTG CTC CTG TCA |
| | | CTC GTG ATA ACA AAG CGA GGC CGG AAA AAA |
| | | TTG CTC TAT ATC TTC AAA CAG CCG TTC ATG |
| | | CGA CCG GTG CAG ACA ACA CAA GAA GAA GAC |
| | | GGC TGC AGC TGC AGA TTC CCT GAG GAG GAA |
| | | GAA GGT GGG TGT GAA TTG AGG GTT AAA TTC |
| | | TCC CGA AGC GCG GAT GCA CCG GCG TAT CAG |
| | | CAG GGC CAA AAT CAA CTC TAC AAC GAG CTC |
| | | AAC CTG GGG CGG CGG GAA GAG TAC GAC GTA |
| | | CTG GAC AAG CGC CGA GGC AGA GAC CCT GAG |
| | | ATG GGG GGC AAG CCT CAA AGG CGA AAG AAC |
| | | CCT CAG GAG GGG CTT TAC AAC GAG CTC CAA |
| | | AAG GAC AAG ATG GQG GAG GCC TAT TCA GAA |
| | | ATC GGC ATG AAA GGC GAA CGA AGG AGA GGT |
| | | AAA GGA CAC GAC GGG CTG TAT CAG GGT TTG |
| | | AGT ACT GCA ACA AAG GAT ACC TAC GAC GCT |
| | | CTC CAC ATG CAA GCG CTG CCC CCA AGG TAG |
| 63 | GDA-501.B: anti- | MDFQVQIFSFLLISASVIMSRGDIQMTQSPSSLSASVGD |
| | Her2 scFv- | RVTITCRASQDVNTAVAWYQQKPGKAPKLLIYSASFLYS |
| | CD8hinge + TM-4- | GVPSRFSGSRSGTDFTLTISSLQPEDFATYYCQQHYTTP |
| | 1BB-CD3zetta | PTFGQGTKVEIKRTGSTSGSGKPGSGEGSEVQLVESGGG |
| | Full AA Seq | LVQPGGSLRLSCAASGFNIKDTYIHWVRQAPGKGLEWVA |
| | | RIYPTNGYTRYADSVKGRFTISADTSKNTAYLQMNSLRA |
| | | EDTAVYYCSRWGGDGFYAMDVWGQGTLVTVSSAATTTPA |
| | | PRPPTPAPTIASQPLSLRPEACRPAAGGAVHTRGLDFAC |
| | | DIYIWAPLAGTCGVLLLSLVITKRGRKKLLYIFKQPFMR |
| | | PVQTTQEEDGCSCRFPEEEEGGCELRVKFSRSADAPAYQ |
| | | QGQNQLYNELNLGRREEYDVLDKRRGRDPEMGGKPQRRK |
| | | NPQEGLYNELQKDKMAEAYSEIGMKGERRRGKGHDGLYQ |
| | | GLSTATKDTYDALHMQALPPR |
| 64 | GDA-501.C: anti- | ATG GAT TTT CAG GTA CAG ATT TTC TCT TTT |
| | Her2 scFv- | CTT CTT ATA AGC GCG TCT GTG ATC ATG AGT |
| | CD28hinge-TM- | CGG GGC GAT ATA CAG ATG ACC CAA AGC CCT |
| | Cy-CD3zetta | TCC TCA CTG TCA GCG AGC GTA GGA GAT AGG |
| | Full Seq codon | GTG ACC ATC ACA TGC AGG GCG AGC CAA GAT |
| | optimization | GTG AAC ACA GCC GTA GCT TGG TAT CAG CAA |
| | | AAG CCA GGC AAG GCT CCC AAA CTG CTG ATA |
| | | TAT TCT GCT AGC TTT CTG TAT TCC GGT GTA |
| | | CCC AGC AGG TTC AGC GGC TCC AGA AGT GGA |
| | | ACC GAC TTC ACT CTG ACA ATC AGT AGC CTT |
| | | CAA CCA GAA GAT TTC GCA ACG TAC TAC TGT |
| | | CAA CAA CAT TAC ACG ACG CCC CCA ACC TTC |
| | | GGG CAA GGA ACG AAA GTT GAG ATA AAA AGG |
| | | ACC GGC AGC ACC TCC GGA AGC GGG AAG CCA |
| | | GGA TCC GGG GAG GGT TCC GAG GTG CAA CTC |
| | | GTC GAG TCA GGT GGC GGT CTC GTG CAA CCA |
| | | GGA GGC TCC CTG CGG CTC TCT TGT GCG GCT |
| | | AGT GGA TTT AAT ATA AAA GAT ACC TAT ATT |
| | | CAC TGG GTG CGC CAA GCA CCT GGA AAA GGG |
| | | CTG GAG TGG GTC GCC AGG ATA TAT CCG ACA |
| | | AAT GGA TAC ACA CGG TAT GCG GAC AGT GTT |
| | | AAA GGC AGG TTC ACA ATT AGC GCA GAC ACG |
| | | AGC AAG AAT ACA GCC TAT CTT CAG ATG AAT |
| | | TCT CTC AGG GCT GAA GAT ACT GCA GTC TAC |
| | | TAT TGC TCT AGG TGG GGT GGT GAC GGC TTT |
| | | TAC GCT ATG GAT GTC TGG GGG CAG GGC ACT |
| | | CTG GTT ACT GTC AGC TCT GCG ATA GAA GTC |
| | | ATG TAC CCT CCG CCG TAT CTT GAC AAT GAG |
| | | AAG TCT AAT GGG ACA ATC ATA CAC GTG AAA |
| | | GGC AAG CAC TTG TGC CCC TCT CCC CTG TTC |
| | | CCC GGC CCT AGT AAA CCG TTC TGG GTG CTC |
| | | GTA GTG GTC GGT GGA GTT CTT GCC TGT TAT |
| | | AGT TTG TTG GTA ACC GTC GCG TTT ATA ATA |
| | | TTC TGG GTC CGG TCC AAG AGA AGC CGC CTC |
| | | CTG CAT TCC GAT TAC ATG AAC ATG ACC CCA |
| | | CGG AGG CCC GGC CCT ACA CGG AAG CAT TAC |
| | | CAG CCA TAC GCT CCG CCT CGA GAT TTT GCT |
| | | GCT TAT AGG TCA CGA GTA AAG TTT AGT AGA |
| | | TCC GCT GAC GCC CCT GCC TAC CAG CAG GGT |
| | | CAG AAT CAG CTC TAC AAT GAA CTG AAC TTG |
| | | GGC AGG CGA GAA GAG TAT GAC GTT CTC GAC |
| | | AAG CGA AGG GGG AGA GAT CCC GAA ATG GGT |
| | | GGT AAA CCA CAA AGA CGC AAG AAT CCT CAG |
| | | GAA GGA TTG TAC AAC GAG CTC CAA AAG GAT |
| | | AAG ATG GQG GAA GCA TAT TCC GAA ATT GGG |
| | | ATG AAG GGC GAG CGA AGG CGG GGC AAA GGA |
| | | CAC GAC GGC CTT TAT CAA GGA CTG TCT ACG |
| | | GCT ACT AAA GAC ACT TAT GAT GCG CTG CAC |
| | | ATG CAA GCA TTG CCG CCG AGA TAG |
| 65 | GDA-501.C: anti- | MDFQVQIFSFLLISASVIMSRGDIQMTQSPSSLSASVGD |
| | Her2 scFv- | RVTITCRASQDVNTAVAWYQQKPGKAPKLLIYSASFLYS |
| | CD28hinge-TM-Cy- | GVPSRFSGSRSGTDFTLTISSLQPEDFATYYCQQHYTTP |
| | CD3zetta Full | PTFGQGTKVEIKRTGSTSGSGKPGSGEGSEVQLVESGGG |
| | Seq | LVQPGGSLRLSCAASGFNIKDTYIHWVRQAPGKGLEWVA |
| | | RIYPTNGYTRYADSVKGRFTISADTSKNTAYLQMNSLRA |
| | | EDTAVYYCSRWGGDGFYAMDVWGQGTLVTVSSAIEVMYP |
| | | PPYLDNEKSNGTIIHVKGKHLCPSPLFPGPSKPFWVLVV |
| | | VGGVLACYSLLVTVAFIIFWVRSKRSRLLHSDYMNMTPR |
| | | RPGPTRKHYQPYAPPRDFAAYRSRVKFSRSADAPAYQQG |
| | | QNQLYNELNLGRREEYDVLDKRRGRDPEMGGKPQRRKNP |
| | | QEGLYNELQKDKMAEAYSEIGMKGERRRGKGHDGLYQGL |
| | | STATKDTYDALHMQALPPR |
| 66 | GDA-501.D: anti- | ATG GAT TTC CAA GTC CAA ATC TTT AGT TTT |
| | Her2 scFv- | TTG CTT ATT TCA GCC TCC GTC ATT ATG AGT |
| | CD8hinge + TM- | AGA GGG GAT ATA CAA ATG ACC CAG TCC CCT |
| | 2B4-CD3zetta | AGT AGC CTG AGC GCA AGC GTT GGA GAC CGG |
| | Full Seq codon | GTA ACC ATA ACA TGT AGG GCT AGC CAG GAC |
| | optimization | GTT AAT ACA GCC GTT GCG TGG TAT CAG CAA |
| | | AAA CCG GGC AAA GCA CCT AAA CTC CTG ATC |
| | | TAT AGC GCC TCA TTC CTG TAT AGC GGC GTC |
| | | CCT AGT CGA TTC TCA GGA AGT AGA TCT GGT |
| | | ACT GAC TTC ACG TTG ACC ATA TCC TCA CTC |
| | | CAA CCC GAG GAC TTC GCA ACA TAT TAT TGC |
| | | CAG CAG CAC TAT ACC ACG CCG CCG ACG TTC |
| | | GGG CAA GGG ACC AAA GTC GAA ATT AAG AGA |
| | | ACA GGG TCT ACG AGT GGC AGT GGT AAA CCC |
| | | GGT TCC GGC GAG GGG TCC GAA GTG CAA TTG |
| | | GTG GAA TCT GGA GGG GGT CTC GTA CAA CCG |
| | | GGG GGC TCC CTT AGA CTC AGT TGT GCC GCG |
| | | AGC GGC TTC AAT ATT AAG GAC ACT TAT ATC |
| | | CAT TGG GTT CGG CAA GCT CCC GGC AAG GGT |
| | | CTT GAA TGG GTA GCA CGC ATA TAC CCG ACA |
| | | AAT GGA TAC ACG CGG TAC GCG GAT AGT GTC |
| | | AAG GGT AGG TTT ACC ATA TCT GCG GAC ACG |
| | | TCC AAA AAC ACC GCC TAC CTT CAG ATG AAT |
| | | TCC CTC CGC GCT GAA GAC ACT GCG GTG TAT |
| | | TAC TGC AGC CGC TGG GGC GGG GAT GGG TTT |
| | | TAC GCC ATG GAT GTG TGG GGT CAG GGT ACA |
| | | CTC GTA ACT GTG AGC AGC GCC ACC ACG ACG |
| | | CCC GCA CCG AGG CCA CCG ACT CCA GCA CCC |
| | | ACT ATC GCT TCT CAA CCT CTG TCA CTG CGC |
| | | CCT GAA GCC TGT CGG CCT GCG GCC GGG GGA |
| | | GCG GTT CAT ACG CGG GGG CTG GAT TTC GCT |
| | | TGC GAC ATT TAT ATA TGG GCA CCC CTC GCG |
| | | GGT ACA TGT GGC GTC CTC TTG CTG AGT CTC |
| | | GTA ATT ACG TGG AGA CGA AAG AGG AAA GAG |
| | | AAG CAG TCT GAA ACT AGC CCT AAG GAG TTC |
| | | CTT ACT ATA TAT GAG GAT GTT AAA GAT CTG |
| | | AAA ACC CGA AGA AAC CAT GAA CAG GAA CAA |
| | | ACT TTC CCT GGC GGG GGA AGT ACG ATT TAC |
| | | AGC ATG ATC CAA TCT CAG TCA AGT GCG CCA |
| | | ACC AGT CAA GAA CCT GCG TAT ACA TTG TAT |
| | | TCC CTC ATT CAG CCA TCT AGG AAA AGC GGT |
| | | TCA CGC AAA AGG AAC CAT AGC CCT TCT TTC |
| | | AAT TCA ACC ATC TAT GAA GTT ATT GGT AAA |
| | | AGT CAA CCT AAA GCG CAA AAT CCG GCG CGA |
| | | CTT AGC CGC AAA GAA CTC GAA AAC TTC GAT |
| | | GTA TAC AGT CGG GTA AAA TTC TCT CGC AGT |
| | | GCC GAT GCG CCT GCC TAC CAG CAG GGA CAG |
| | | AAT CAG TTG TAC AAC GAA CTG AAC CTC GGC |
| | | CGA AGA GAG GAG TAT GAT GTT CTT GAT AAG |
| | | CGG AGG GGC AGA GAT CCC GAG ATG GGT GGG |
| | | AAG CCA CAA AGA CGA AAA AAT CCT CAG GAG |
| | | GGA CTG TAC AAT GAA CTT CAA AAA GAC AAG |
| | | ATG GCT GAA GCC TAC TCT GAG ATT GGG ATG |
| | | AAA GGT GAG CGC CGA AGG GGA AAG GGC CAC |
| | | GAC GGC TTG TAT CAA GGA CTG TCC ACC GCC |
| | | ACT AAA GAT ACG TAC GAT GCC TTG CAT ATG |
| | | CAA GCC CTT CCT CCA CGC TAG |
| 67 | GDA-501.D: anti- | MDFQVQIFSFLLISASVIMSRGDIQMTQSPSSLSASVGD |
| | Her2 scFv- | RVTITCRASQDVNTAVAWYQQKPGKAPKLLIYSASFLYS |
| | CD8hinge + TM- | GVPSRFSGSRSGTDFTLTISSLQPEDFATYYCQQHYTTP |
| | 2B4-CD3zetta | PTFGQGTKVEIKRTGSTSGSGKPGSGEGSEVQLVESGGG |
| | Full AA Seq | LVQPGGSLRLSCAASGFNIKDTYIHWVRQAPGKGLEWVA |
| | | RIYPTNGYTRYADSVKGRFTISADTSKNTAYLQMNSLRA |
| | | EDTAVYYCSRWGGDGFYAMDVWGQGTLVTVSSATTTPAP |
| | | RPPTPAPTIASQPLSLRPEACRPAAGGAVHTRGLDFACD |
| | | IYIWAPLAGTCGVLLLSLVITWRRKRKEKQSETSPKEFL |
| | | TIYEDVKDLKTRRNHEQEQTFPGGGSTIYSMIQSQSSAP |
| | | TSQEPAYTLYSLIQPSRKSGSRKRNHSPSFNSTIYEVIG |
| | | KSQPKAQNPARLSRKELENFDVYSRVKFSRSADAPAYQQ |
| | | GQNQLYNELNLGRREEYDVLDKRRGRDPEMGGKPQRRKN |
| | | PQEGLYNELQKDKMAEAYSEIGMKGERRRGKGHDGLYQG |
| | | LSTATKDTYDALHMQALPPR |
| 68 | CXCR4R^{334X} | ATG GAG GGT ATT TCA ATT TAT ACC TCC GAC |
| | Full Sequence | AAT TAC ACG GAG GAA ATG GGA AGT GGA GAT |
| | Codon | TAC GAC TCC ATG AAA GAG CCA TGC TTT AGG |
| | Optimization | GAA GAG AAC GCC AAC TTT AAT AAG ATC TTT |
| | | CTC CCG ACA ATT TAT TCT ATT ATC TTC CTC |
| | | ACC GGC ATT GTC GGA AAT GGT CTT GTT ATT |
| | | CTT GTT ATG GGC TAT CAA AAG AAA CTT CGA |
| | | TCT ATG ACT GAT AAG TAC AGG CTT CAC CTC |
| | | AGT GTC GCC GAT CTG TTG TTT GTA ATA ACT |
| | | CTT CCG TTT TGG GCG GTA GAT GCC GTT GCT |
| | | AAC TGG TAC TTC GGT AAC TTC CTT TGT AAA |
| | | GCT GTA CAT GTC ATT TAC ACA GTT AAT CTG |
| | | TAT AGC AGC GTG CTG ATA CTG GCG TTT ATT |
| | | TCA CTG GAC CGC TAC CTC GCC ATC GTT CAC |
| | | GCC ACG AAT AGT CAG CGA CCG CGG AAG CTC |
| | | TTG GCC GAG AAG GTT GTG TAC GTG GGC GTG |
| | | TGG ATT CCC GCT CTG CTC TTG ACA ATT CCC |
| | | GAC TTC ATA TTT GCC AAT GTG AGC GAA GCC |
| | | GAT GAT CGC TAC ATC TGT GAT AGA TTC TAT |
| | | CCG AAT GAT TTG TGG GTA GTA GTT TTT CAA |
| | | TTC CAA CAC ATC ATG GTG GGC CTG ATC TTG |
| | | CCG GGA ATC GTA ATC CTG AGT TGT TAC TGC |
| | | ATT ATT ATC TCT AAG TTG TCC CAC TCA AAG |
| | | GGC CAT CAG AAG CGC AAG GCG TTG AAG ACG |
| | | ACT GTT ATA CTC ATA TTG GCA TTC TTT GCC |
| | | TGT TGG CTG CCT TAC TAT ATT GGG ATA TCT |
| | | ATA GAT TCT TTC ATA CTC CTG GAG ATC ATA |
| | | AAG CAG GGA TGT GAA TTT GAA AAT ACT GTA |
| | | CAT AAG TGG ATT TCC ATA ACT GAA GCG CTT |
| | | GCG TTC TTT CAT TGT TGT CTG AAT CCA ATT |
| | | CTC TAT GCG TTT CTG GGG GCA AAA TTT AAA |
| | | ACC TCA GCG CAA CAT GCA CTG ACC AGT GTA |
| | | TCA CGG GGC TCT AGC CTT AAA ATA CTT TCC |
| | | AAA GGC AAG TAA |
| 69 | CXCR4R^{334X} | MEGISIYTSDNYTEEMGSGDYDSMKEPCFREENANFNKI |
| | Amino Acid | FLPTIYSIIFLTGIVGNGLVILVMGYQKKLRSMTDKYRL |
| | Sequence | HLSVADLLFVITLPFWAVDAVANWYFGNFLCKAVHVIYT |
| | | VNLYSSVLILAFISLDRYLAIVHATNSQRPRKLLAEKVV |
| | | YVGVWIPALLLTIPDFIFANVSEADDRYICDRFYPNDLW |
| | | VVVFQFQHIMVGLILPGIVILSCYCIIISKLSHSKGHQK |
| | | RKALKTTVILILAFFACWLPYYIGISIDSFILLEIIKQG |
| | | CEFENTVHKWISITEALAFFHCCLNPILYAFLGAKFKTS |
| | | AQHALTSVSRGSSLKILSKGKX |

1. A population of nucleated cells comprising a plurality of nature killer (NK) cells, wherein the population comprises at least 1.0×10⁶ nucleated cells, wherein at least about 70% of the NK cells in the population are viable and express Receptor Linker IL-15, wherein:
at least about 85% of cells in the population are CD56+;
no more than about 0.5% of the cells in the population are CD3+;
no more than about 10% of the cells in the population are CD19+;
no more than about 10% of the cells in the population are CD14+;
no more than about 20% of the cells in the population are LAG3+;
at least about 10% of the cells in the population are CD122+; and
no more than about 20% of the cells in the population are NKG2A+.

2. The population of nucleated cells of claim 1, wherein the Receptor Linker IL-15 is SEQ ID NO: 25 or SEQ ID NO: 28.

3. The population of nucleated cells of claim 1, wherein at least about 75% of the NK cells that express Receptor Linker IL-15, and optionally, wherein said 75% of the NK cells also do not express CISH.

4. A population of nucleated cells comprising a plurality of NK cells, wherein the population comprises at least 1.0×10⁶ nucleated cells, wherein at least about 70% of the NK cells in the population are viable and express anti-CD38 chimeric antigen receptor (CAR), wherein:
At least about 85% of cells in the population are CD56+;
no more than about 0.5% of the cells in the population are CD3+;
no more than about 10% of the cells in the population are CD19+;
no more than about 10% of the cells in the population are CD14+;
no more than about 15% of the cells in the population are CD38+; and
wherein said CAR comprises an anti-CD38 scFv.

5.-7. (canceled)

8. The population of nucleated cells claim 4, wherein the CAR comprises:
(i) a hinge domain selected from CD28 and CD8, optionally
(ii) a transmembrane domain selected from CD28, CD8, and NKG2D, optionally
(iii) a co-stimulatory domain selected from CD28, 4-1BB, 2B4, CD3zetaR, OX40, Lsk, ICOS, DAP10, and Fc fragment of IgE receptor 1g, optionally
(iv) an activation domain is selected from CD32, FcR-γ, and Fc-epsilon-R, and optionally
(v) wherein the CAR comprises a signal peptide.

9.-12. (canceled)

13. The population of nucleated cells of claim 4, wherein the CAR is selected from SEQ ID NO: 31 and SEQ ID NO: 32.

14. The population of nucleated cells of claim 1, wherein the cells that express Receptor Linker IL-15 or anti CD38 CAR, further comprise a mutant chemokine receptor, and optionally wherein the mutant chemokine receptor is SEQ ID NO: 69.

15. (canceled)

16. A method of ex vivo producing genetically modified natural killer (NK) cells of claim 1, the method comprising:
(a) downregulating expression of a gene of interest in a population of NK cells so as to obtain a population of NK cells having been genetically modified to down-regulate a gene of interest;
(b) expanding said population of NK cells having been genetically modified to down-regulate a gene of interest so as to obtain an ex vivo expanded population of NK cells; and
(c) upregulating expression of at least one membrane bound protein in said ex vivo expanded population of NK cells, thereby producing the genetically modified NK cells.

17. A method of ex vivo producing natural killer (NK) cells expressing at least one membrane bound protein of claim 1, the method comprising:
(a) expanding a population of NK cells by a method comprising:
(i) culturing said population of NK cells under conditions allowing for cell proliferation, wherein said conditions comprise providing an effective amount of nutrients, serum, IL-15 and nicotinamide; and
(ii) supplementing said population of NK cells with an effective amount of fresh nutrients, serum, IL-15 and nicotinamide 5-10 days following step (i) to produce expanded NK cells;
so as to obtain an ex vivo expanded population of NK cells, and
(b) upregulating expression of at least one membrane bound protein in said ex vivo expanded population of NK cells, thereby producing the NK cells expressing the at least one membrane bound protein.

18. The method of claim 16, wherein said population of NK cells is derived from cord blood, peripheral blood, bone marrow, CD34+ cells or iPSCs, optionally wherein said population of NK cells is deprived of CD3⁺ cells, and optionally wherein said population of NK cells comprises CD3 CD56⁺ cells.

19.-20.

21. The method of claim 16, wherein said downregulating is effected by a gene editing system, wherein said NK cells are in a culture and wherein said downregulating is affected 24-72 hours from initiation of said culture.

22.-23. (canceled)

24. The method of claim 16, wherein said gene of interest comprises a gene whose product effects proliferation and/or survival of said NK cells, and, optionally, wherein said gene of interest is selected from the group consisting of CISH, TGFβ receptor and CD38.

25. (canceled)

26. The method of claim 16, wherein said expanding said population of NK cells is affected under conditions allowing for cell proliferation, wherein:
(i) said conditions comprise an effective amount of nutrients, serum, growth factors and nicotinamide;
(ii) said growth factors comprise at least one growth factor selected from the group consisting of IL-15, IL-2, IL-7, IL-12, IL-21, SCF and FLT3, and/or
(iii) wherein said effective amount of said nicotinamide comprises an amount between 1.0 mM to 10 mM, and/or
(iv) wherein said expanding said population of NK cells is affected in the presence of feeder cells or a feeder layer, and/or
(v) wherein said expanding said population of NK cells is affected in the presence of feeder cells or a feeder layer, and/or
(vi) wherein said expanding said population of NK cells is affected in the presence of feeder cells or a feeder layer, optionally wherein said feeder cells comprise irradiated cells and/or wherein said feeder cells comprise T cells or PBMCs, and/or a CD3 agonist.

27.-32. (canceled)

33. The method of claim 16, wherein said expanding said population of NK cells is affected for 14-16 days.

34. The method of claim 21, wherein said upregulating expression of said at least one membrane bound protein is affected on day 12-14 from initiation of culture.

35. The method of claim 21, wherein said upregulating expression of said at least one membrane bound protein is affected by mRNA electroporation and, optionally, wherein said at least one membrane bound protein is transiently expressed.

36. (canceled)

37. An isolated population of NK cells obtainable according to the method of claim 16,
wherein said population of NK cells comprises a plurality of natural killer (NK) cells, wherein the population comprises at least 1.0×10⁶ nucleated cells, wherein at least about 70% of the NK cells in the population are viable and express Receptor Linker IL-15, wherein;
at least about 85% of cells in the population are CD56+;
no more than about 0.5% of the cells in the population are CD3+;
no more than about 10% of the cells in the population are CD19+;
no more than about 10% of the cells in the population are CD14+;
no more than about 20% of the cells in the population are LAG3+;
at least about 10% of the cells in the population are CD122+, and
no more than about 20% of the cells in the population are NKG2At.

38. A pharmaceutical composition comprising the isolated population of NK cells of claim 37 and a pharmaceutically active carrier.

39. A method of treating a disease in a subject in need thereof, the method comprising administering to the subject a therapeutically effective amount of the isolated population of NK cells of claim 37, thereby treating the subject.

40. (canceled)

41. The method of claim 39, wherein the disease is selected from the group consisting of a malignant disease, a viral disease, a bacterial disease, a fungal disease, a protozoa disease, and a parasite disease, and optionally:
(i) wherein said malignant disease is a solid tumor or tumor metastasis,
(ii) wherein said malignant disease is selected from the group consisting of a breast cancer, an ovarian cancer, a bladder cancer, a pancreatic cancer, a stomach cancer, a lung cancer, a melanoma, a sarcoma, a neuroblastoma, a colon cancer, a colorectal cancer, an esophageal cancer, a synovial cell cancer, a uterus cancer, a glioma and a cervical cancer,
(iii) wherein said malignant disease is a hematological malignancy, and wherein said hematological malignancy comprises a leukemia, a lymphoma or multiple myeloma.

42.-46. (canceled)
